(19) United States
(12) Patent Application Publication
(10) Pub. No.: US 2026/0240985 A1
(43) Pub. Date: Aug. 20, 2026

(54) ORAL VACCINE

(71) Applicant: WESTFÄLISCHE WILHELMS-UNIVERSITÄT MÜNSTER, Münster (DE)

(72) Inventors: Joachim JOSE, Münster (DE); Florian LENZ, Münster (DE); Yvonne BÖRGELING, Münster (DE)

(73) Assignee: WESTFÄLISCHE WILHELMS-UNIVERSITÄT MÜNSTER, Münster (DE)

(21) Appl. No.: 19/159,291

(22) PCT Filed: Feb. 27, 2024
(86) PCT No.: PCT/EP2024/054988

§ 371 Date: Aug. 22, 2025

(30) Foreign Application Priority Data
Feb. 27, 2023 (LU) LU503544

(51) Int. Cl. A61K 39/215; A61K 39/00; A61K 39/015; A61K 39/12

(52) U.S. Cl. CPC A61K 39/215; A61K 39/015; A61K 39/12; A61K 2039/523; A61K 2039/542; A61K 2039/55516

(57) ABSTRACT

The present invention relates to a non-viable bacterium cell wherein an immunogenic polypeptide fused to an auto-transporter comprising transmembrane linker and a trans-porter domain is displayed on the surface of the cell, and to a preparation comprising such non-viable cells. The preparation is useful as an oral vaccine.

The present invention relates to a non-viable bacterium cell wherein an immunogenic polypeptide fused to an autotransporter comprising transmembrane linker and a trans-porter domain is displayed on the surface of the cell, and to a preparation comprising such non-viable cells. The preparation is useful as an oral vaccine.

Jong et al. (Microbial Cell Factories 13 (2014), 162) describe an autotransporter dis-play platform for the development of a recombinant bacterial vector vaccine. They demonstrate secretion and display of Mycobacterium tuberculosis antigens in E. coli simultaneously. Furthermore, they show stable multivalent display of these antigens in an attenuated Salmonella typhimurium strain upon chromosomal integration and demonstrate expression of multiple antigenic fragments from Chlamydia trachomatis and the influenza A virus at the Salmonella cell surface. They propose to use this autotransporter display platform for the generation of multivalent recombinant bacterial live vaccines but also for derived non-living vaccines based on outer membrane vesi-cles or bacterial ghosts. An autotransporter-based antigen display in bacterial ghosts obtained by expression of the lysis gene E is described by Hjelm et al. (Appl. Env. Microbiol. 81 (2015), 726-735).

The vaccines proposed by Jong et al. or Hjelm et al., however, suffer from substantial disadvantages. On the one hand, administration of a bacterial live vaccine is principally associated with potential health risks. On the other hand, administration of outer mem-brane vesicles or bacterial ghosts may be less effective than administration of intact bacterium cells. Further, no evidence of a long-term immunogenicity and stability was demonstrated.

Thus, it was an object of the present invention, to provide a highly effective, stable and safe vaccine.

According to a first aspect, the present invention relates to a non-viable bacterium cell wherein an immunogenic polypeptide fused to an autotransporter protein comprising transmembrane linker and a transporter domain is displayed on the surface of the cell.

In certain embodiments, the immunogenic polypeptide is attached to the surface of the outer membrane of a Gram-negative bacterium cell by a β-barrel autotrans-porter, which is serving as an anchor within the outer membrane. In certain embod-iments, the immunogenic polypeptide is located N-terminally to the β-barrel auto-transporter. In further embodiments, the immunogenic polypeptide is located C-ter-minally to the β-barrel autotransporter.

In particular embodiments, the immunogenic polypeptide is from a coronavirus, e.g., MERS, SARS-COV-1, or SARS-COV-2, or from a Plasmodium organism, e.g., Plasmodium falciparum, from a human papillomavirus (HPV), or from a bovine viral diarrhea virus (BVDV).

In some embodiments, the bacterium cell does not contain a gene encoding an antibiotic resistance polypeptide.

In particular embodiments, the bacterium cell contains a metabolic selection marker gene.

In particular embodiments, the non-viable bacterium cell is lyophilized.

A further aspect of the present invention is a preparation comprising non-viable bacte-rium cells as described above wherein the preparation is free from living bacterial cells.

In particular embodiments, the preparation is a dried preparation, e.g., a lyophilized preparation.

In further particular embodiments, the preparation is an effervescent powder.

In some embodiments, the preparation comprises bacterium cells that do not contain a gene encoding an antibiotic resistance polypeptide.

In particular embodiments, the preparation comprises bacterium cells that contain ametabolic selection marker gene.

In particular embodiments, the non-viable bacterium cell or the preparation is suitable for pharmaceutical use.

Still a further aspect of the present invention is an oral vaccine comprising the non-viable bacterium cell or the preparation as described above.

In certain embodiments, the bacterium cell is a Gram-negative bacterium cell. In par-ticular embodiments, the bacterium cell is a Gram-negative bacterium cell, e.g., a Sal-monella cell, an E. coli cell, or a Vibrio cholerae cell, which is suitable for oral admin-istration to a subject, e.g., a human subject.

In a specific embodiment, the bacterium cell is a Salmonella typhi Ty 21a cell (Germa-nier& Fürer, J. Infect. Dis. 131 (1975), 553-558; Bumann et al., Vaccine 20 (2002), 845-852; Kopecko et al., Int. J. Med. Microbiol. 209 (2009), 233-246) the contents of which are herein incorporated by reference. This embodiment also includes a genetic variant, particularly a genetic variant that is deficient in a gene encoding a polypeptide essential for producing and/or degrading a metabolic product in the cell, e.g., a ΔthyA (thymidylate synthase) variant.

In a further specific embodiment, the bacterium cell is an E. coli Nissle 1917 cell (Schultz, Inflammatory Bowel Dis. 14 (2008), 1012-1018; Yu et al., Microb. Biotechnol. 13 (2020), 629-636) the contents of which are herein incorporated by reference This embodiment also includes a genetic variant, particularly a genetic variant that is defi-cient in a gene encoding a polypeptide essential for producing and/or degrading ametabolic product in the cell, e.g., a ΔthyA variant.

In a further specific embodiment, the bacterium cell is a Vibrio cholerae Vaxchora cell (Cabrera et al, Ann. Pharmacother. 51 (2017), 584-589) the content of which is herein incorporated by reference This embodiment also includes a genetic variant, particu-larly a genetic variant that is deficient in a gene encoding a polypeptide essential for producing and/or degrading a metabolic product in the cell, e.g., a ΔthyA variant.

The immunogenic polypeptide displayed on the surface of the bacterium cell is a portion of a recombinant autotransporter fusion protein located in the outer mem-brane of the host cell. The recombinant autotransporter protein typically comprises (i): an N-terminal signal peptide (which may be cleaved off), (ii) a passenger domain, which is an immunogenic polypeptide, and (iii) an autotransporter protein comprises-inga linker domain, and a β-barrel domain. The passenger domain can be located N-terminally or C-terminally to the autotransporter protein domain.

In certain embodiments, the recombinant autotransporter protein comprises in di-rection from the N-terminus to the C-terminus (i): a signal peptide (which may be cleaved off), (ii) a passenger domain, which is an immunogenic polypeptide, and (iii) an autotransporter protein comprising a linker domain and a transporter domain, particularly a β-barrel domain. In these embodiments, the autotransporter is typically a classical autotransporter such as EhaA. In further embodiments, the recombinant autotransporter protein comprises in direction from the N-terminus to the C-terminus (i): a signal peptide (which may be cleaved off), (ii) an autotransporter protein com-prising a transporter domain, particularly a β-barrel domain, and a linker, and (iii) a passenger domain, which is an immunogenic polypeptide. In these embodiments, the autotransporter is typically an inverse autotransporter such as YeeJ.

The autotransporter fusion protein is encoded by a recombinant nucleic acid molecule which is extrachromosomally or chromosomally present in the bacterium cell. The re-combinant nucleic acid molecule is operatively linked to an expression control se-quence including a promoter and optionally further sequences required for gene ex-pression in the respective host cell. The expression control sequence may be ho-mologous or heterologous to the host cell. Preferably, the nucleic acid molecule is located on a recombinant vector, e.g., a plasmid vector. The skilled person knows expression control sequences, in particular for expression in the host cell species as described herein.

In certain embodiment, the recombinant nucleic molecule is operatively linked to an inducible promoter, e.g., the arabinose-inducible araBAD promoter (Greenfield et al. Proc. Natl. Acad. Sci. USA 75 (1978), 4724-4728) the content of which is herein incorporated by reference. In further embodiments, the inducible promoter is a rhamnose promoter, a Lac promoter, a Tac promoter, a pRox promoter (Meyers et al., Microb Biotechnol, 12 (2019), 1003-1013. Alternatively, a constitutive promoter such as a pGB35 type promoter (Zobel et al., ACS Synthetic Biology, 4 (2015), 1341-1351) may be used.

The sequence of the nucleic acid molecule can have a codon usage adapted to the host cell. In particular, the codon usage of the transmembrane linker sequence, the transporter domain or/and the passenger can be adapted to the host cell. More par-ticular, the codon usage of the transmembrane linker sequence or/and the trans-porter domain can be adapted to the host cell. This can improve expression if the transporter domain is heterologous to the host cell. Optimisation of codon usage does usually not affect the amino acid sequence.

In certain embodiments, the recombinant nucleic acid molecule comprises:
(i) a portion encoding a signal peptide,
(ii) a portion encoding the immunogenic polypeptide to be displayed,
(iii) a portion encoding an autotransporter comprising a transmem-brane linker and a transporter domain.

In further embodiments, the recombinant nucleic acid molecule comprises:
(i) a portion encoding a signal peptide,
(ii) a portion encoding an autotransporter comprising a transporter domain and a transmembrane linker; and
(iii) a portion encoding the immunogenic polypeptide to be displayed.

The recombinant nucleic acid molecule comprises a portion encoding an N-terminal signal peptide, preferably a portion coding for a Gram-negative signal peptide allow-ing for transport into the periplasm through the inner cell membrane. The signal peptide may be a signal peptide homologous to the host cell. The signal peptide may also be a signal peptide heterologous to the host cell. An example of a suitable signal peptide is the cholera toxin B (CtxB) signal peptide. In further embodiments, a PelB, OmpA, YeeJ or OprF signal peptide may be used. The signal peptide can be cleaved off during maturation of the polypeptide.

The recombinant nucleic acid molecule further comprises a portion encoding an immunogenic polypeptide. The term “immunogenic polypeptide” refers to any polypep-tide capable of raising an immune response, e.g., an antibody response and/or a T-cell response, in a host organism to which the bacterium cell is administered. The host organism is typically a vertebrate organism, e.g., a mammal including a human.

In certain embodiments, the immunogenic polypeptide is a polypeptide from a pathogen, e.g., a virus, bacterium, fungus, or parasite. In particular embodiments, the immunogenic polypeptide is from a pathogen selected from a coronavirus, e.g., MERS, SARS-COV-1, or SARS-COV-2, a Plasmodium organism, e.g., Plasmodium falcipa-rum, Mycobacterium tuberculosis, Mycobacterium leprae, Trypanosoma brucei, Tryp-anosoma cruzei, Neisseria menigitidis, Haemophilus influenzae, Streptococcus pneu-moniae, Dengue virus, Ebola virus, or from a human papillomavirus (HPV), preferably a tumorigenic HPV-type such as HPV-type 6, 11, 16, 18, 31, 33, 45, 52, and 58, or from a bovine viral diarrhea virus (BVDV).

In further embodiments, the pathogen is multi-drug resistant bacterium such as Staph-ylococcus aureus (MRSA), E. coli EHEC, Enterobacter, or Streptococcus pneu-moniae. In still further embodiments, the pathogen is selected from Borrelia burgdorferi or Campylobacter jejunii.

In even more particular embodiments, the immunogenic polypeptide is a SARS-COV-2 spike glycoprotein or an immunogenic fragment thereof, a SARS-COV-2 receptor binding domain or an immunogenic fragment thereof optionally including a trimeriza-tion domain, or a SARS-COV-2 nucleocapsid protein or an immunogenic fragment thereof.

In further even more particular embodiments, the immunogenic polypeptide is a sur-face-associated 6-cysteine domain of Plasmodium falciparum sexual stadium protein

Pfsi230 or an immunogenic fragment thereof, in particular Pfs230D1M, a surface associated circumsporozoid-protein of Plasmodium falciparum or an immunogenic frag-ment thereof, in particular RV21.

In further even more particular embodiments, the immunogenic polypeptide is an L1 protein of a human papillomavirus or an immunogenic fragment thereof, in particular HPV L1 from HPV-type 18 or another tumorigenic HPV type such as HPV type 16, 31, 33, 35, 39, 45, 51, 52, 56, 58 and 59.

In further even more particular embodiments, the immunogenic polypeptide is a bovine viral diarrhea virus E2 protein or an immunogenic fragment thereof, in particular BVDV344.

In a specific embodiment, the immunogenic polypeptide is a SARS-COV-2 nucleocap-sid protein comprising amino acids 40-457 of SEQ ID NO: 2, or a polypeptide having an identity of at least 70%, at least 90%, at least 95% or at least 98% thereto over the whole sequence. The immunogenic polypeptide may be encoded by the nucleotide sequence of nucleotides 118-1371 of SEQ ID NO: 1 or a nucleotide sequence within the scope of the degeneracy of the genetic code.

In a further specific embodiment, the immunogenic polypeptide is a SARS-COV-2 spike protein comprising amino acids 40-1237 of SEQ ID NO: 4, or a polypeptide hav-ing an identity of at least 70%, at least 90%, at least 95% or at least 98% thereto over the whole sequence. The immunogenic polypeptide may be encoded by the nucleotide sequence of nucleotides 118-3711 of SEQ ID NO: 3 or a nucleotide sequence within the scope of the degeneracy of the genetic code.

In a further specific embodiment, the immunogenic polypeptide is a SARS-COV-2 re-ceptor binding domain (RBD) comprising amino acids 40-262 of SEQ ID NO: 5 or 7,or a polypeptide having an identity of at least 80%, at least 90% or at least 95% thereto over the whole sequence. The immunogenic polypeptide may be encoded by the nu-cleotide sequence of nucleotides 118-786 of SEQ ID NO: 6 or 8 or a nucleotide se-quence within the scope of the degeneracy of the genetic code.

In certain embodiments, the immunogenic polypeptide, e.g., the SARS-COV-2 RBD, may comprise a multimerization domain, e.g., a dimerization or trimerization domain at its N- or C-terminus. In a specific embodiment, the multimerization domain is a tri-merization domain comprising amino acids 263-289 of SEQ ID NO: 5, or a polypeptide having an identity of at least 70%, at least 90%, at least 95% or at least 98% thereto over the whole sequence. The trimerization domain may be encoded by the nucleotide sequence of nucleotides 787-867 of SEQ ID NO: 6 or a nucleotide sequence within the scope of the degeneracy of the genetic code.

In a further specific embodiment, the immunogenic polypeptide is a Plasmodium falciparum Pfs 230 D1M polypeptide comprising amino acids 1-195 of SEQ ID NO: 9 or a polypeptide having an identity of at least 70%, at least 90%, at least 95% or at least 98% thereto over the whole sequence.

In a further specific embodiment, the immunogenic polypeptide is a Plasmodium falciparum RV21 polypeptide comprising amino acids 1-268 of SEQ ID NO: 10 or a polypeptide having an identity of at least 70%, at least 90%, at least 95% or at least 98% thereto over the whole sequence.

In a further specific embodiment, the immunogenic polypeptide is a HPV L1 polypeptide comprising amino acids 1-507 of SEQ ID NO: 11 or a polypeptide having an identity of at least 70%, at least 90%, at least 95% or at least 98% thereto over the whole sequence.

In a further specific embodiment, the immunogenic polypeptide is the N-terminal fragment of the BVDV E2 polypeptide comprising amino acids 1-344 of SEQ ID NO: 12 or a polypeptide having an identity of at least 70%, at least 90%, at least 95% or at least 98% thereto over the whole sequence.

The skilled person knows suitable methods to determine the degree of identity of nu-cleic acid sequences and amino acid sequences. Known algorithms, such as NBLAST (for nucleic acids) or BLASTp (for amino acid sequences) may be used. A nucleic acid or polypeptide comprising sequences having at least 70%, at least 80%, at least 90%, at least 95%, at least 98%, or at least 99% identity to a given sequence over the whole sequence or fragments of the given nucleic acid or polypeptide.

The recombinant nucleic acid molecule further comprises a portion encoding an au-totransporter which is located C-terminally or N-terminally to the portion encoding the immunogenic polypeptide.

This portion comprises a transmembrane linker which is required for the presentation of the passenger polypeptide on the outer surface of the outer membrane of the host cell. The transmembrane linker is typically located between the autotransporter do-main and the immunogenic polypeptide.

A transmembrane linker domain may be used which is homologous with regard to the autotransporter domain, i.e., the transmembrane linker domain is encoded by a nucleic acid portion directly 3′ or 5′ to the autotransporter domain. In further embodiments, a transmembrane linker domain may be used which is heterologous with regard to the autotransporter domain. The length of the transmembrane linker is preferably 30-250 amino acids. The transmembrane linker is preferably a transmembrane linker obtained from an EhaA protein as described herein. In a specific embodiment, the transmem-brane linker comprises amino acids 484-687 of SEQ ID NO: 1, or a polypeptide having an identity of at least 70%, at least 90%, at least 95% or at least 98% thereto over the whole sequence. The transmembrane linker may be encoded by the nucleotide sequence of nucleotides 1450-2061 of SEQ ID NO: 2 or a nucleotide sequence within the scope of the degeneracy of the genetic code.

The recombinant nucleic acid molecule further comprises a portion encoding the transporter domain of an autotransporter protein. Examples of suitable autotrans-porter proteins are described by Celik et al. (PLOS ONE 7 (2012), e43245; Leo et al. (Int J Med Microbiol. 305 (2015), 276-82) and Meuskens et al. (Front Microbiol. 31 (2019), 1163) the contents of which are herein incorporated by reference.

In certain embodiments, the autotransporter protein is AIDA-I (Jose et al., Microbiol. Mol. Biol. 71 (2007), 600-619) or haemoglobin protease (Hbp) (Jong et al., supra) the contents of which are herein incorporated by reference.

In certain embodiments, the autotransporter protein is an inverse autotransporter such as YeeJ, intimin, or invasin (Tian et al., Microb. Biotechnol. 15 (2022), 2235-2249; Martinez-Gil et al., Sci Rep. 7 (2017), 11326) the contents of which are herein incorporated by reference.

In particular embodiments, the autotransporter protein is EhaA, an autotransporter protein derived from E. coli strain 0157:H7 (Wells et al., Environ. Microbiol. 10 (2008), 589-604) the content of which is herein incorporated by reference.

In a specific embodiment, the autotransporter domain is an EhaA autotransporter β-barrel domain comprising amino acids 688-972 of SEQ ID NO: 2, or a polypeptide having an identity of at least 70%, at least 90%, at least 95% or at least 98% thereto over the whole sequence. The autotransporter domain may be encoded by the nucle-otide sequence of nucleotides 2062-2916 of SEQ ID NO: 1 or a nucleotide sequence within the scope of the degeneracy of the genetic code.

The EhaA transporter domain also includes variants which can be obtained by al-tering the amino acid sequence in the loop structures of the β-barrel not participating in the transmembrane portions. Optionally, the nucleic acid portions coding for the surface loops can be deleted completely. Also, within the amphipathic β-sheet con-served amino exchanges, i.e., the exchange of a hydrophilic by another hydrophilic amino acid or/and the exchange of a hydrophobic by another hydrophobic amino acid may take place.

The recombinant nucleic acid molecule can further comprise at least one nucleic acid sequence encoding an affinity tag. The nucleic acid sequence encoding the affinity tag can flank the portion encoding the immunogenic polypeptide to be displayed. For ex-ample, the affinity tag may be an His-epitope tag, a Myc tag and/or a PEYVK tag. In this embodiment, the nucleic acid sequence encoding the affinity tag can be separated from the portion encoding the immunogenic polypeptide by a sequence encoding at least one protease recognition sequence, e.g., a recognition sequence for a bacterial protease, and/or a recognition sequence for a eukaryotic, particularly a mammalian protease. For example, the at least one protease recognition sequence is inde-pendently selected from a factor Xa cleavage site, an OmpT cleavage site, and a TEV protease cleavage site.

Further, the recombinant nucleic acid molecule can further comprise at least one re-striction enzyme recognition site for facilitate the cloning of the portion encoding the immunogenic polypeptide to be displayed.

In certain embodiments, the non-viable bacterium cell comprises a first immunogenic polypeptide and a second immunogenic polypeptide which is different from the first immunogenic polypeptide. In particular embodiments, first immunogenic polypeptide and the second immunogenic polypeptide may be from a pathogen as described above, e.g., from the same pathogen or are from different pathogens. In these embod-iments, the first and the second immunogenic polypeptides may be separately fused to a first and a second autotransporter, and optionally the first autotransporter is differ-ent from the second autotransporter.

In further particular embodiments, first immunogenic polypeptide is from a pathogen and the second immunogenic polypeptide is an adjuvant polypeptide wherein the first immunogenic polypeptide is different from the second immunogenic polypeptide. In these embodiments, the first and the second immunogenic polypeptides may be sep-arately fused to a first and a second autotransporter, and optionally the first autotrans-porter is different from the second autotransporter, and optionally the first autotrans-porter is different from the second autotransporter. Alternatively, the extracellular por-tion of the autotransporter itself may comprise the second adjuvant immunogenic pol-ypeptide.

Examples of adjuvant polypetides used herein are e.g., cholera toxin or a subunit thereof, such as cholera toxin B subunit, enterotoxin or a subunit thereof, such as a subunit of type Ilb heat-labile enterotoxin (LT-Ilb-B5), Brucella cell-surface protein 31 (rBCSP31), endopeptidase O (PepO), and/or DnaJ as described by Holmgren et al., Immunol Lett. 97 (2) (2005), 181-188; Li et al., Cell. Mol. Immunol. 11 (2014), 477-494; Liang et al., Vaccine 27 (2009), 4302-4308; Zhang et al., Front. Cell. Infect. Microbiol. 6 (2016), 23 and Su et al., Front. Immunol. 8 (2017), 686, the contents of which are herein incorporated by reference.

In a particular embodiment, the present invention relates to a mixture of one or more different non-viable bacterium cells. In certain embodiments, the mixture comprises a first non-viable bacterium cell comprising a first immunogenic polypeptide and a second non-viable bacterium cell comprising a second immunogenic polypeptide wherein the first immunogenic polypeptide is different from the second immunogenic polypeptide. In particular embodiments, first immunogenic polypeptide and the sec-ond immunogenic polypeptide may be from a pathogen as described above, e.g., from the same pathogen or are from different pathogens. In these embodiments, the first and the second immunogenic polypeptides are separately fused to a first and a second autotransporter, and optionally the first autotransporter is different from the second autotransporter.

In further particular embodiments, first immunogenic polypeptide is from a pathogen and the second immunogenic polypeptide is an adjuvant polypeptide as described above.

The recombinant nucleic acid molecule encoding the fused polypeptide may be located on an extrachromosomal vector, e.g., a plasmid.

The recombinant bacterium cell may comprise a selection marker gene which is useful for propagation. In certain embodiments, the selection marker gene is located on an extrachromosomal vector, e.g., the vector which also harbors the recombinant nucleic acid molecule.

In certain embodiments, the vector contains a metabolic selection marker gene, such as an antibiotic resistance gene, e.g., an ampicillin resistance gene or a kan-amycin resistance gene.

In further embodiments, the bacterium cell does not contain a gene encoding an antibiotic resistance polypeptide. In these embodiments, the bacterium cell may be auxotrophic with regard to an essential metabolic product, in particular thymidylate, aspartate etc. Thus, in a preferred embodiment, the vector containing the recombinant nucleic acid molecule as described herein does not contain an antibiotic resistance gene, but an essential gene, e.g. a gene encoding an enzyme required for providing an essential metabolic product for the bacterium cell, in particular thymidylate syn-thase, aspartate-semialdehyde dehydrogenase, KDPG-aldolase, orotidine-5′-phos-phate decarboxylase/pyrroline-5-carboxylate reductase, as described by Bumann et al., Vaccine 20 (5-6) (2001), 845-852; Curtis et al., Res Microbiol. 141 (7-8) (1990), 797-805; Xin et al., Infect Immun. 80 (10) (2012), 3621-33; Voss and Steinbüchel, Metab Eng. 8 (1) (2006), 66-78 and Schneider et al., Biotechnol Prog. 21 (2) (2005), 343-8, the contents of which are herein incorporated by reference.

In particular, the essential gene is deleted from the chromosomal DNA and provided on an extrachromosomal vector, resulting in cells requiring the vector to survive. Growth of the bacterium cell containing said vector indicates incorporation of the plasmid into the bacterium cell.

The non-viable bacterium cell may be prepared by a process comprising:
(a) cultivating a viable bacterium cell in a culture medium wherein the bacterium cell is transformed or transfected with a recombinant nucleic acid molecule comprising:
   (i) a portion encoding a signal peptide,
   (ii) a portion encoding the immunogenic polypeptide to be displayed,
   (iii) a portion encoding an autotransporter comprising a transmem-brane linker and the transporter domain of an autotransporter protein, or
   (i) a portion encoding a signal peptide,
   (ii) a portion encoding an autotransporter comprising the transporter domain of an autotransporter protein and a transmembrane linker; and
   (iii) a portion encoding the immunogenic polypeptide to be displayed, under conditions where the recombinant nucleic acid molecule is expressed;
(b) obtaining a bacterium cell wherein an immunogenic polypeptide fused to an auto-transporter protein comprising transmembrane linker and an autotransporter domain encoding bay the recombinant nucleic acid molecule of (a) is displayed on the surface of the cell;
(c) subjecting the bacterium cell of (b) to conditions where a non-viable bacterium cell is obtained.

Steps (a) and (b) as described above may be performed by standard methods known in the art. The person skilled in the art knows suitable culture conditions allowing for an efficient expression of passenger proteins on the surface of host cells, particularly E. coli or other Gram-negative bacterial cells up to 100 000 or more molecules per cell by using a liquid medium of the following composition: 5 g/l to 20 g/l, preferably about 10 g/l tryptone, 2 g/l to 10 g/l, preferably about 5 g/l yeast extract, 5 g/l to 20 g/l, in particular about 10 g/l NaCl and the remaining part water. The medium should possibly contain as little as possible divalent cations. The liquid medium may contain in addition preferably EDTA in a concentration of 2 μM to 20 μM, in particular 10 μM. Moreover, it contains preferably reducing reagents, such as 2-mercaptoethanol, dithiothreitol or dithioerythritol in a preferred concentration of 2 mM to 20 mM. The reducing reagents favour a non-folded structure of the polypeptide during transport. The liquid medium can further contain additional C-sources, preferably glucose, e.g. in an amount of up to 10 g/l, in order to favour secretion i.e. transfer of the passenger to the surrounding medium.

In particular embodiments, the expression system as described herein employing the EhaA autotransporter domain is termed MATE (maximized autotransporter expres-sion) system of pMATE system. Plasmids, in particular expression plasmids comprises-ing the nucleic acid fusion as described herein to be used in the MATE system are also termed by the prefix “pMATE”. The MATE/pMATE system is described in WO 2014/139862, the content of which is herein incorporated by reference.

Step (c) comprises subjecting the bacterium cell to conditions wherein the bacterium cell is rendered non-viable. In certain embodiments, the bacterium cell is rendered non-viable by lyophilization. In particular embodiments, step (c) comprises lyophilizing the bacterium cell of (b) in the presence of a phosphate ion containing aqueous me-dium, e.g., a phosphate-buffered saline (PBS) containing aqueous medium. In certain embodiments, the lyophilization comprises freezing a suspension of the cells to a tem-perature of about −80° C., e.g. for a time period of at least 6 h or overnight. In certain embodiments, cells are kept after freezing for at least 1 d, e.g., at least 2 d at a tem-perature of −40° C. or −80° C.

In further embodiments, the non-viable bacterium cell may be obtained by UV irradia-tion, heating or treatment of an intact bacterium cell with chemical agent such as NaNO₂ or beta-propiolactone.

In embodiments, wherein mixtures of different non-viable bacterium cells comprising a first immunogenic polypeptide and non-viable bacterium cells comprising a second immunogenic polypeptide are prepared, the individual components of the mixture may be separately obtained in step (b), and mixed before lyophilization or after lyophiliza-tion in step (c).

The bacterium cell of the invention is a non-viable cell. In certain embodiments, the non-viable bacterium cell is an intact cell, e.g., a cell which comprises a cell membrane and cytoplasmic components wherein the cell membrane may be permeable. Accord-ing to flow cytometric measurements, the morphology of a non-viable cell after lyoph-ilization is similar or identical to a viable cell.

In certain embodiments, the bacterium cell of the invention is a lyophilized non-viable bacterium cell. In particular embodiments, the cell has been lyophilized in a phosphate and/or sodium, potassium and chloride containing medium, e.g., a PBS (phosphate-buffered saline) buffer, i.e., 137 mM NaCl, 2.7 mM KCl, 10 mM Na₂HPO₄, 2.0 mM KH₂PO₄, pH 7.4 as described herein. Surprisingly, the bacterium cell lyophilized un-der these conditions is non-viable, i.e., it cannot be recultivated by a procedure as described herein. After incubation for 16 or 24 h at 37° C., no cell growth was observed.

A further aspect of the present invention is a preparation comprising non-viable bacte-rium cells as described above, particularly lyophilized and optionally reconstituted non-viable bacterium cells, wherein the preparation is free from living bacterial cells. This is an advantageous feature for medical applications, particularly for applications in-volving an oral delivery. In certain embodiments, the preparation comprises about 10⁸ to 10¹² cells.

In certain embodiments, the preparation is a solid preparation, e.g., a tablet or a powder. In particular embodiments, the preparation is an effervescent powder. In further embodiments, the preparation is liquid preparation, e.g., an aqueous or oily suspension.

In certain embodiments, the preparation comprises bacterium cells and additional components from the lyophilization procedure, for example phosphate ions, and/or sodium and chloride ions. In certain embodiments, the preparation further comprises phosphate ions, sodium ions and chloride ions.

In certain embodiments, the preparation further comprises at least one pharmaceutically acceptable excipient, e.g., a filler, diluent, lubricant etc. Those excipients are typically added after the lyophilization procedure. Examples of suitable excipients include excipients known to the skilled person from tablet or powder formulations, e.g., cellulose, or cellulose derivatives, lactose, starch, sorbitol, and/or mannose. In particular embodiments, the preparation comprises an acidic compound and a carbonate or hydrogen carbonate containing compound for providing an effervescent preparation.

The preparation of the invention is stable, i.e., it retains immunogenicity when stored over an extended time period. In certain embodiments, it retains at least 50%, at least 70% or at least 90% immunogenicity when measured in vitro in suitable antigen test.

In particular embodiments, the preparation is stable as a lyophilizate at a temperature of 25° C., particularly at a temperature of 25° C. in a closed vial for at least 5 months or for at least 12 months. In further particular embodiments, the preparation is stable as a lyophilizate at a temperature of 37° C., particularly at a temperature of 37° C. in a closed vial for at least 1 month or for at least 12 months.

The non-viable bacterium cell or the preparation is suitable for pharmaceutical use. In certain embodiments, the non-viable bacterium cell or the preparation is useful as a vaccine, particularly an oral vaccine. In particular embodiments, the preparation is use-ful as a vaccine for immunization a subject against an infection by a pathogen, partic-ularly the pathogen from which the immunogenic polypeptide originates. Presentation of the immunogenic polypeptide on the surface of a bacterium cell is expected to pro-vide a strong immune response in a vaccinated subject, e.g., a vaccinated human subject since the components of the bacterium cell, particularly the autotransporter components presented on the cell surface are effective as an inherent adjuvant.

Thus, a further aspect of the present invention is a vaccine comprising the non-viable bacterium cell or the preparation as described herein. The inventors have found that the immunogenicity of the immunogenic peptides is preserved over a long time period even at room temperature or higher. Further, the inventos have found that administer-ing the preparation comprising the non-viable bacterium cells in a dried form, particu-larly in a lyophilize form provides an additional immunostimulatory effect.

The vaccine may comprise a single type of non-viable bacterium cell or a plurality of different types of non-viable bacterium cells, i.e., bacterium cells presenting different immunogenic polypeptides on their surface. The different immunogenic polypeptides may be from a single pathogen or from different pathogensas described above.

In a preferred embodiment, the vaccine is a polyvalent HPV vaccine, e.g., a nonavalent HPV vaccine that protects against HPV types 6, 11, 16, 18, 31, 33, 45, 52 and 58, or a quadrivalent HPV vaccine that protects against HPV types 6, 11, 16 and 18, or a divalent HPV vaccine that protects against HPV types 16 and 18.

The present invention is further explained by the following examples.

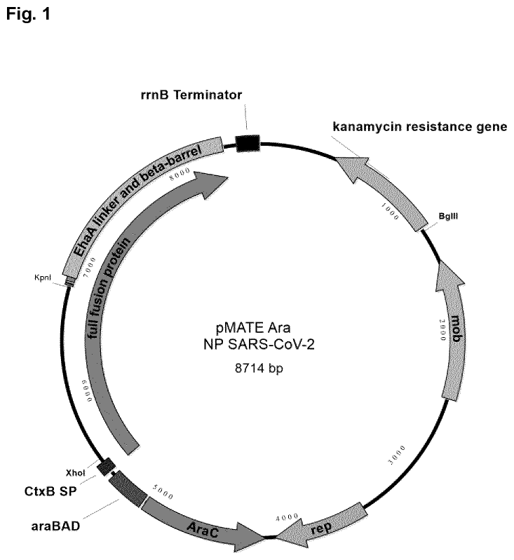

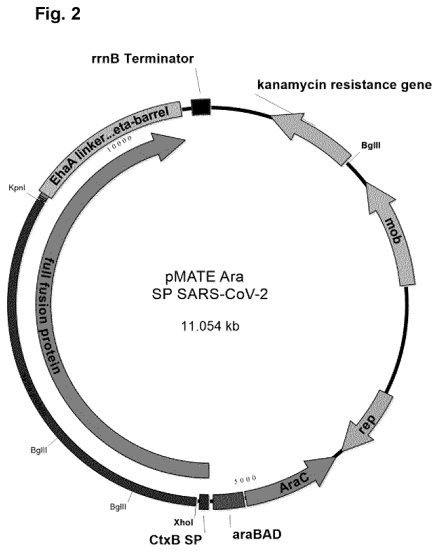

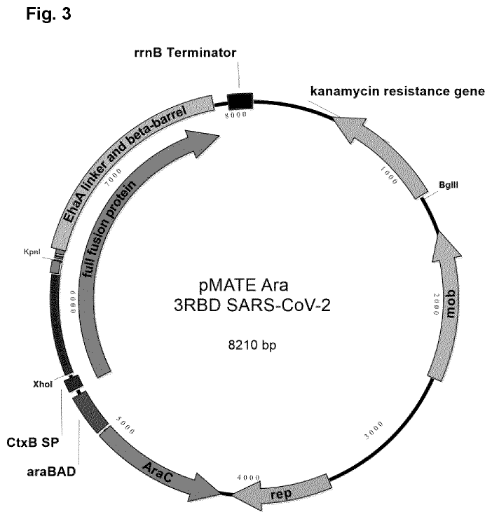

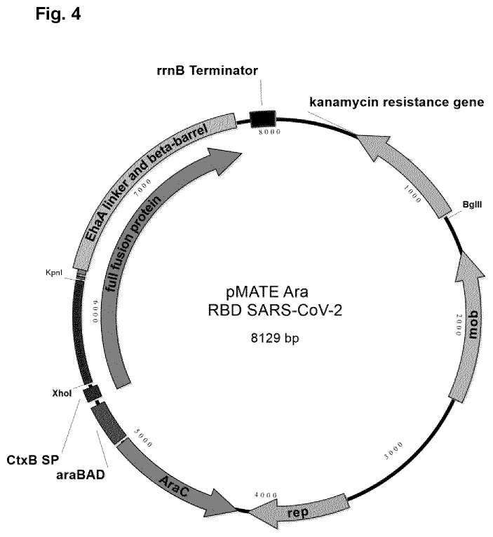

According to the methods described in WO 2014/139862, pMATE expression plas-mids were constructed. FIG. 1 shows the plasmid pMATE Ara NP SARS-COV2, FIG. 2 shows the plasmid pMATE Ara SP SARS-COV2, FIG. 3 shows the plasmid pMATE Ara 3RBD SARS-COV2 (including the trimerization domain), and FIG. 4 shows the plasmid pMATE Ara RBD SARS-COV2 (without trimerization domain).

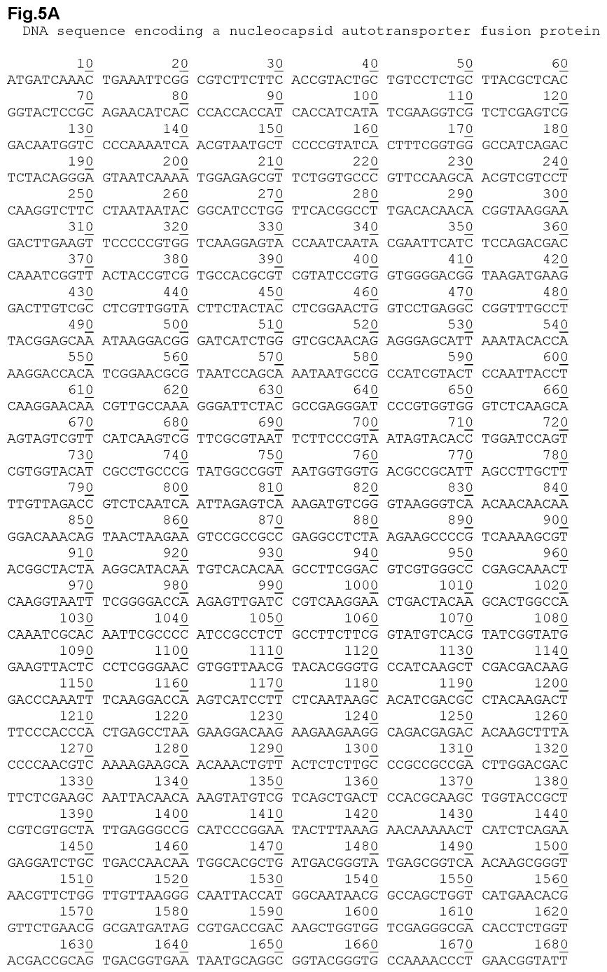

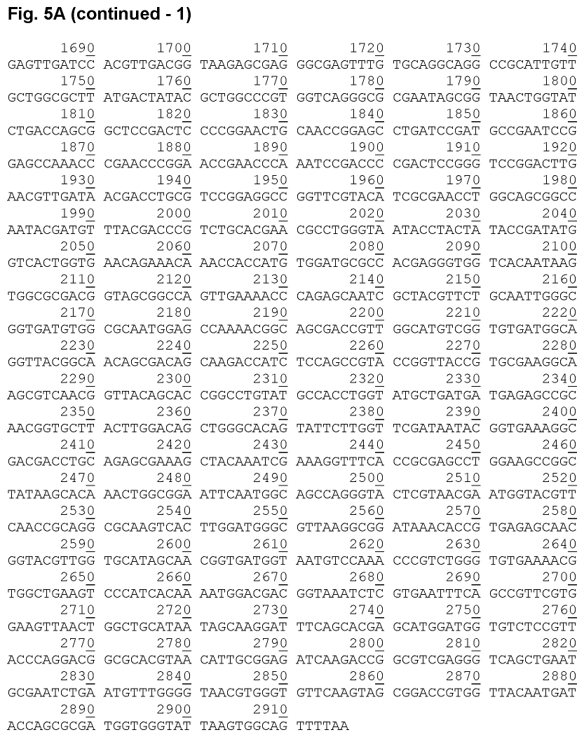

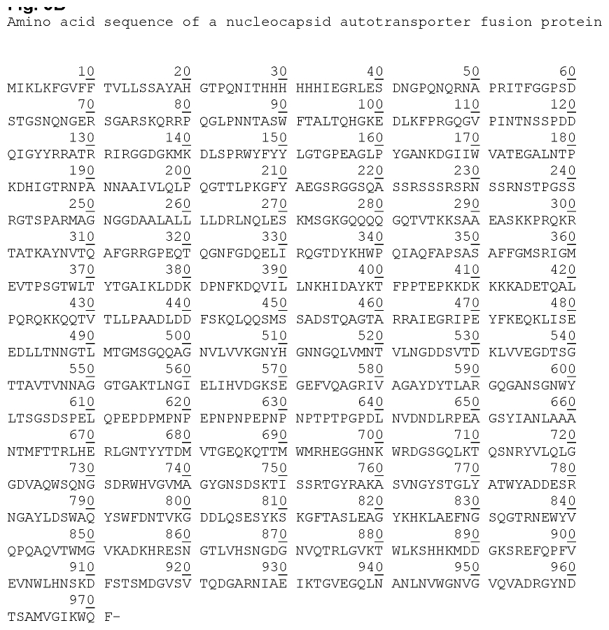

FIG. 5a shows the nucleotide sequence encoding the nucleocapsid autotransporter fusion protein from pMATE Ara NP SARS-COV2 (SEQ ID NO: 1).

### Annotation of SEQ ID NO:1

| feature | position |
|---|---|
| CtxB signal peptide sequence | 1-81 bp |
| 6xHis Tag | 82-99 bp |
| Factor Xa cleavage site | 100-111 bp |
| Xhol restriction site | 112-117 bp |
| Nucleocapsid protein | 118-1371 bp |
| Kpnl restriction site | 1372-1377 bp |
| OmpT cleavage site | 1378-1389 bp |
| Factor Xa cleavage site | 1390-1401 bp |
| PEYFK-Tag | 1405-1419 bp |
| Myc-Tag | 1420-1449 bp |
| EhaA Linker | 1450-2061 bp |
| β-Barrel | 2062-2916 bp |

FIG. 5b shows the amino acid sequence of the nucleocapsid autotransporter fusion protein (SEQ ID NO: 2).

### Annotation of SEQ ID NO:2

| feature | position |
|---|---|
| CtxB signal peptide sequence | 1-27 aa |
| 6xHis Tag | 28-33 aa |
| Factor Xa cleavage site | 34-37 aa |
| Xhol restriction site | 38-39 aa |
| Nucleocapsid protein | 40-457 aa |
| Kpnl restriction site | 458-459 aa |
| OmpT cleavage site | 460-463 aa |
| Factor Xa cleavage site | 464-467 aa |
| PEYFK-Tag | 469-473 aa |
| Myc-Tag | 474-483 aa |
| EhaA Linker | 484-687 aa |
| β-Barrel | 688-971 aa |

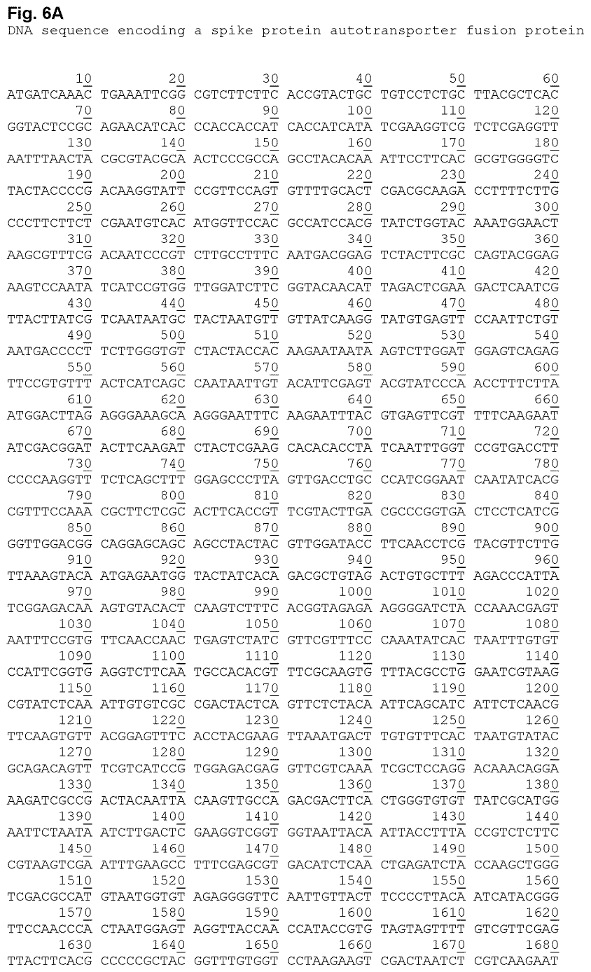

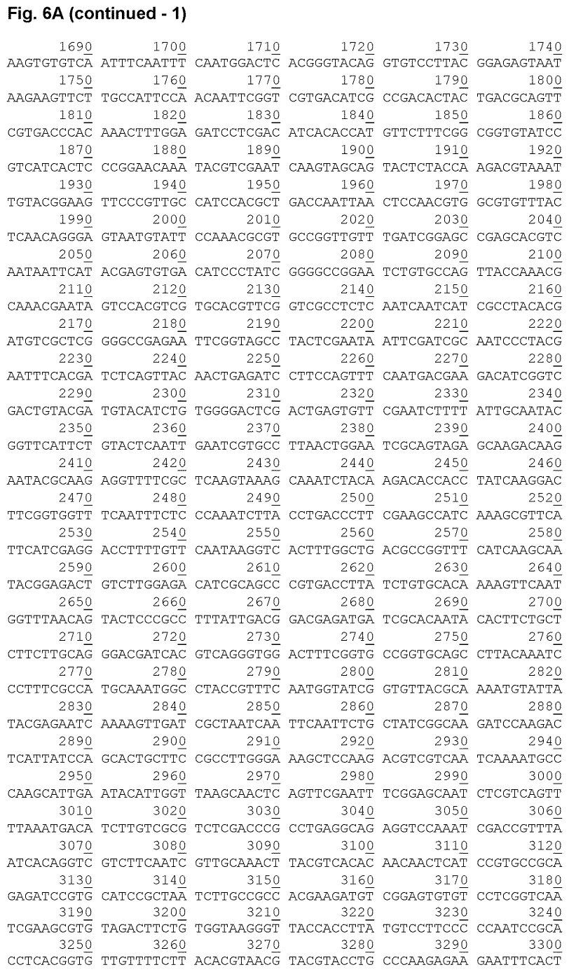

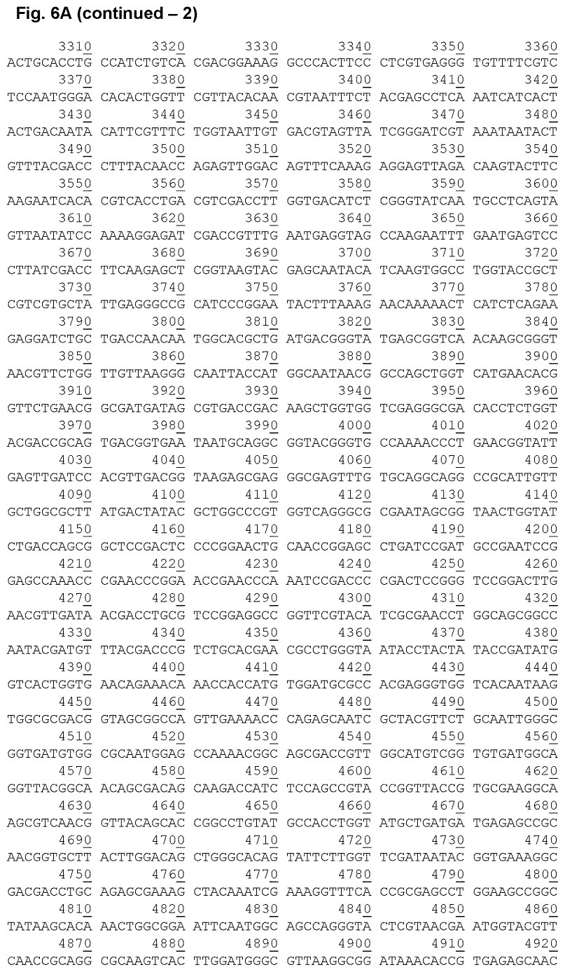

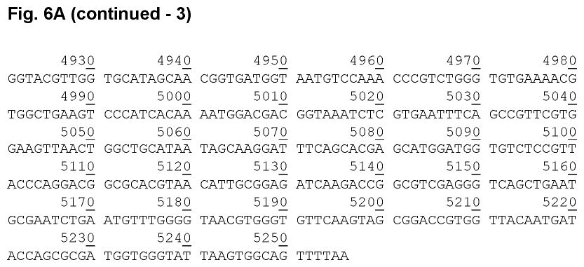

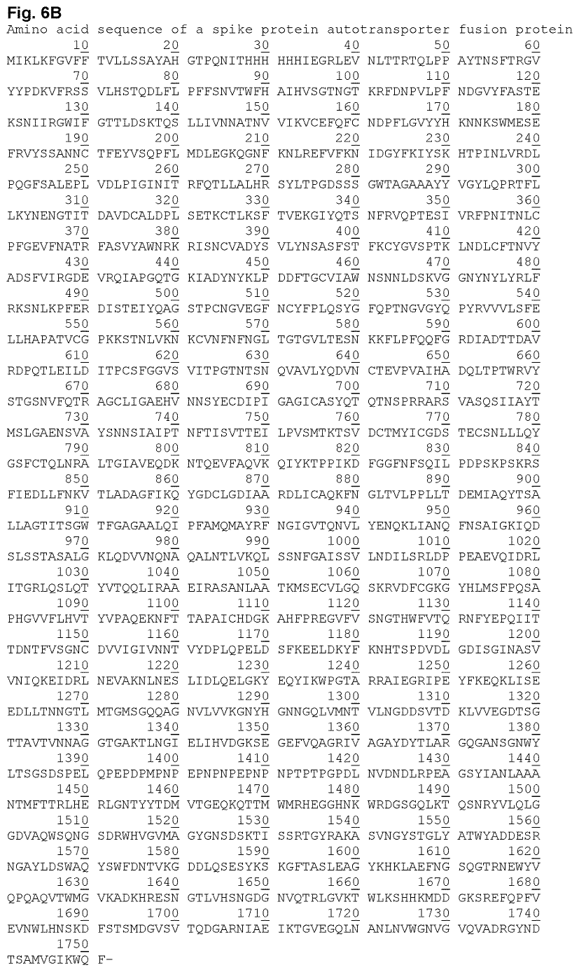

FIG. 6a shows the nucleotide sequence encoding the spike autotransporter fusion pro-tein from pMATE Ara SP SARS-COV2 (SEQ ID NO: 3).

### Annotation of SEQ ID NO:3

| feature | position |
|---|---|
| CtxB signal peptide sequence | 1-81 bp |
| 6xHis Tag | 82-99 bp |
| Factor Xa cleavage site | 100-111 bp |
| Xhol restriction site | 112-117 bp |
| Spike protein | 118-3711 bp |
| Kpnl restriction site | 3712-3717 bp |
| OmpT cleavage site | 3718-3729 bp |
| Factor Xa cleavage site | 3730-3741 bp |
| PEYFK-Tag | 3745-3759 bp |
| Myc-Tag | 3760-3789 bp |
| EhaA Linker | 3790-4401 bp |
| β-Barrel | 4402-5256 bp |

FIG. 6b shows the amino acid sequence of the spike autotransporter fusion protein (SEQ ID NO: 4).

### Annotation of SEQ ID NO:4

| feature | position |
|---|---|
| CtxB signal peptide sequence | 1-27 aa |
| 6xHis Tag | 28-33 aa |
| Factor Xa cleavage site | 34-37 aa |
| Xhol restriction site | 38-39 aa |
| Spike protein | 40-1237 aa |
| Kpnl restriction site | 1238-1339 aa |
| OmpT cleavage site | 1240-1243 aa |
| Factor Xa cleavage site | 1244-1247 aa |
| PEYFK-Tag | 1249-1253 aa |
| Myc-Tag | 1254-1263 aa |
| EhaA Linker | 1264-1467 aa |
| β-Barrel | 1468-1751 aa |

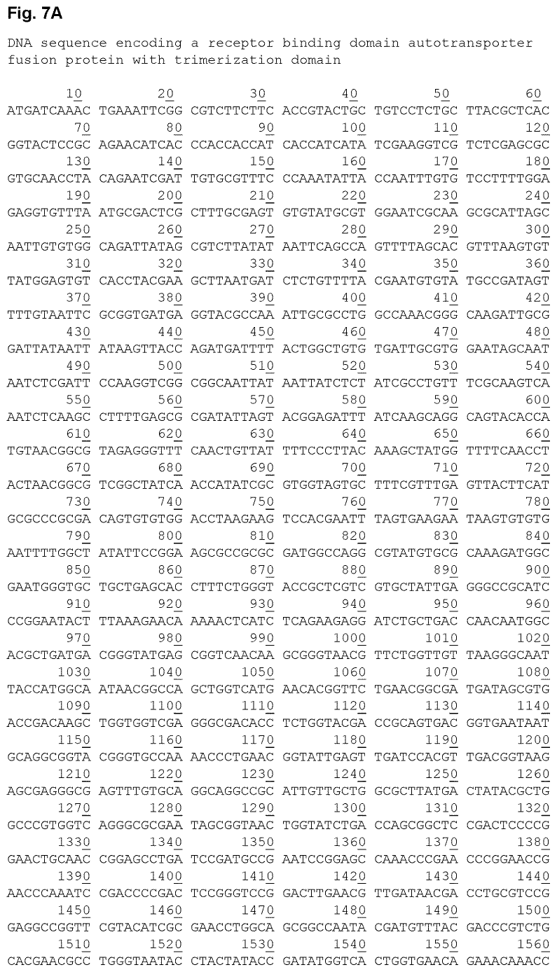

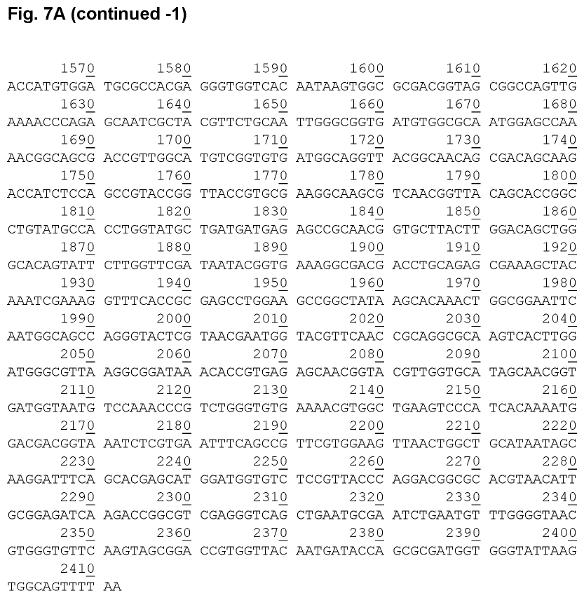

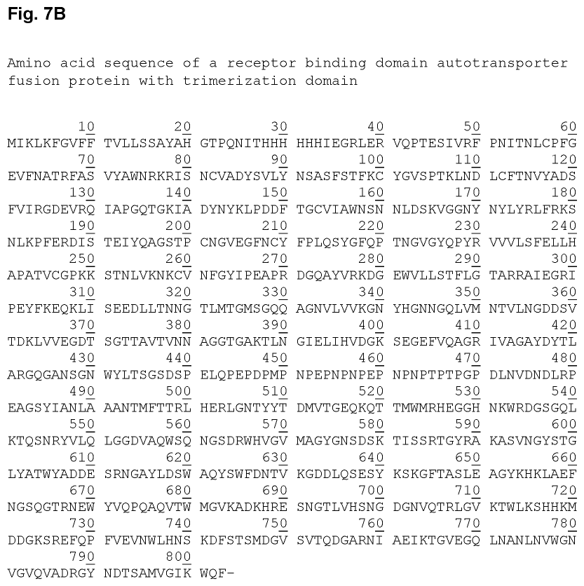

FIG. 7a shows the nucleotide sequence encoding the receptor binding domain (RBD) autotransporter fusion protein including the trimerization domain from pMATE Ara 3RBD SARS-COV2 (SEQ ID NO: 5).

### Annotation of SEQ ID NO:5

| feature | position |
|---|---|
| CtxB signal peptide sequence | 1-81 bp |
| 6xHis Tag | 82-99 bp |
| Factor Xa cleavage site | 100-111 bp |
| Xhol restriction site | 112-117 bp |
| RBD protein | 118-786 bp |
| Trimerization domain | 787-867 bp |
| Kpnl restriction site | 868-873 bp |
| OmpT cleavage site | 874-885 bp |
| Factor Xa cleavage site | 886-897 bp |
| PEYFK-Tag | 901-915 bp |
| Myc-Tag | 916-945 bp |
| EhaA Linker | 946-1557 bp |
| β-Barrel | 1558-2411 bp |

FIG. 7b shows the amino acid sequence of the receptor binding domain autotrans-porter fusion protein including the trimerization domain (SEQ ID NO: 6).

### Annotation of SEQ ID NO:6

| feature | position |
|---|---|
| CtxB signal peptide sequence | 1-27 aa |
| 6xHis Tag | 28-33 aa |
| Factor Xa cleavage site | 34-37 aa |
| Xhol restriction site | 38-39 aa |
| RBD protein | 40-262 aa |
| Trimerization domain | 263-289 aa |
| Kpnl restriction site | 290-291 aa |
| OmpT cleavage site | 292-295 aa |
| Factor Xa cleavage site | 296-299 aa |
| PEYFK-Tag | 301-305 aa |
| Myc-Tag | 306-315 aa |
| EhaA Linker | 316-519 aa |
| β-Barrel | 520-803 aa |

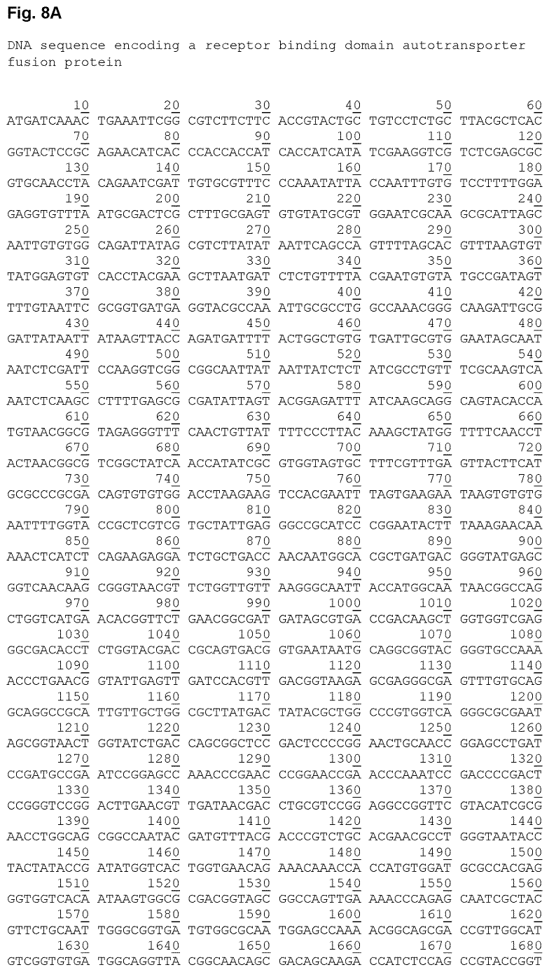

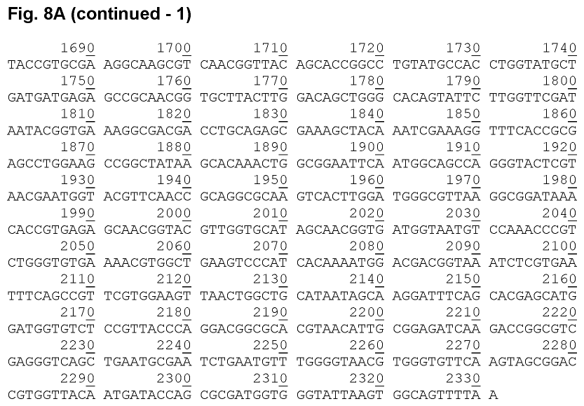

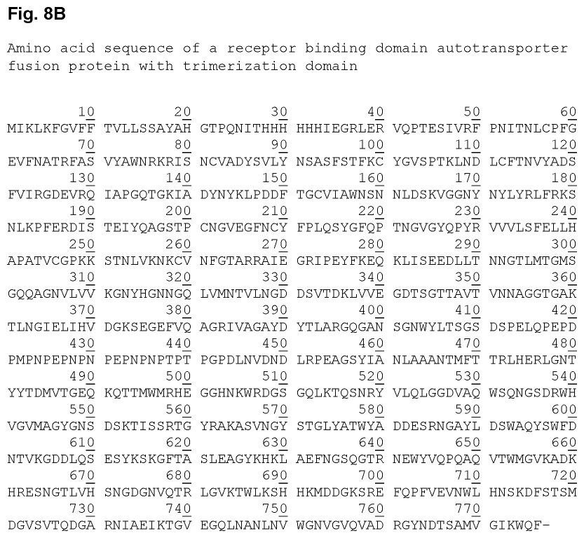

FIG. 8a shows the nucleotide sequence encoding the receptor binding domain auto-transporter fusion protein without trimerization domain from pMATE Ara RBD SARS-CoV2 (SEQ ID NO: 7).

### Annotation of SEQ ID NO:7

| feature | position |
|---|---|
| CtxB signal peptide sequence | 1-81 bp |
| 6xHis Tag | 82-99 bp |
| Factor Xa cleavage site | 100-111 bp |
| Xhol restriction site | 112-117 bp |
| RBD protein | 118-786 bp |
| Kpnl restriction site | 787-792 bp |
| OmpT cleavage site | 793-804 bp |
| Factor Xa cleavage site | 805-816 bp |
| PEYFK-Tag | 820-834 bp |
| Myc-Tag | 835-864 bp |
| EhaA Linker | 867-1476 bp |
| β-Barrel | 1477-2331 bp |

FIG. 8b shows the amino acid sequence of the receptor binding domain autotrans-porter fusion protein without trimerization domain (SEQ ID NO: 8).

### Annotation of SEQ ID NO:8

| feature | position |
|---|---|
| CtxB signal peptide sequence | 1-27 aa |
| 6xHis Tag | 28-33 aa |
| Factor Xa cleavage site | 34-37 aa |
| Xhol restriction site | 38-39 aa |
| RBD protein | 40-262 aa |
| Kpnl restriction site | 263-264 aa |
| OmpT cleavage site | 265-268 aa |
| Factor Xa cleavage site | 269-272 aa |
| PEYFK-Tag | 274-278 aa |
| Myc-Tag | 279-288 aa |
| EhaA Linker | 289-492 aa |
| β-Barrel | 493-776 aa |

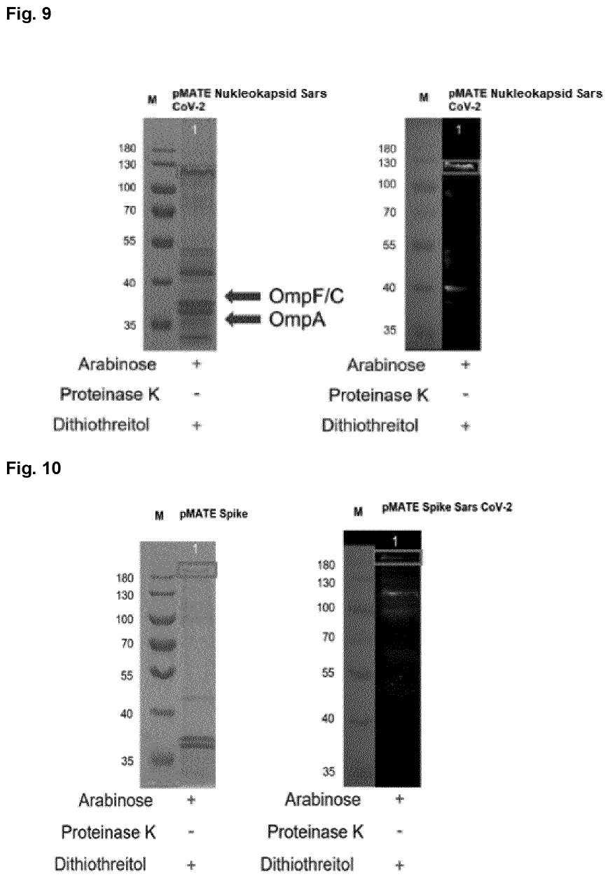

FIG. 9 shows the expression and surface display of SARS COV-2 nucleocapsid pro-tein in S. typhi Ty21a in a Coomassie gel (left) and a Western blot (right). The nu-cleocapsid protein is depicted in the box of lane 1. Arabinose was added to induce expression of the fusion protein. Proteinase K was added to digest surface-exposed proteins.

FIG. 10 shows the expression and surface display of SARS COV-2 spike protein in S. typhi Ty21a in a Coomassie gel (left) and a Western blot (right). The spike protein is depicted in the box of lane 1. Arabinose was added to induce expression of the fusion protein. Proteinase K was added to digest surface-exposed proteins.

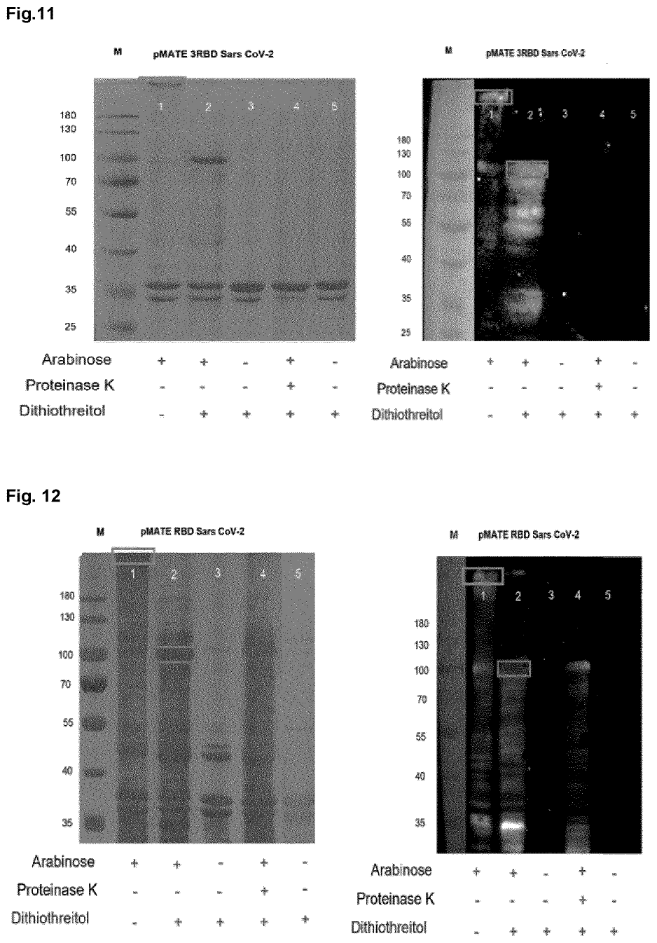

FIG. 11 shows the expression and surface display of SARS COV-2 3RBD with tri-merization domain in S. typhi Ty 21a in a Coomassie gel (left) and a Western blot (right). The trimeric RBD (w/o dithiothreitol) is depicted in the box of lane 1. The monomeric RBD (with dithiothreitol) is depicted in the box of lane 2. Arabinose was added to induce expression of the fusion protein. Proteinase K was added to digest surface-exposed proteins.

FIG. 12 shows the expression and surface display of SARS COV-2 RBD with trimer-ization domain in S. typhi Ty 21a in a Coomassie gel (left) and a Western blot (right). The trimeric RBD (w/o dithiothreitol) is depicted in the box of lane 1. The monomeric RBD (with dithiothreitol) is depicted in the box of lane 2. Arabinose was added to induce expression of the fusion protein. Proteinase K was added to digest surface-exposed proteins.

The S. typhi cells expressing the recombinant fusion proteins were harvested and lyophilized.

The lyophilization procedure was performed as follows:
cultivating bacterium cells in a shaker flask at 200 rpm and 37° C. in 20 ml soy LB medium with 50 ng/ml kanamycin to an OD₅₇₈ of 0.5;
adding 0.2% (w/v) arabinose and incubating the cells for additional 2 h at 200 rpm and 37° C.;
centrifuging the cells at 5,000 rpm and 4° C. for 5 min;
suspending the cells in phosphate-buffered saline and diluting to an OD₅₇₈ of 10;
freezing the cells overnight to −80° C.; and
lyophilizing the cells at a temperature of −40° C., e.g., for 4 d under a vacuum of 0.220 mbar in a freezer (Alpha 1-2LD plus, Martin Christ, Osterode am Harz, Germany).

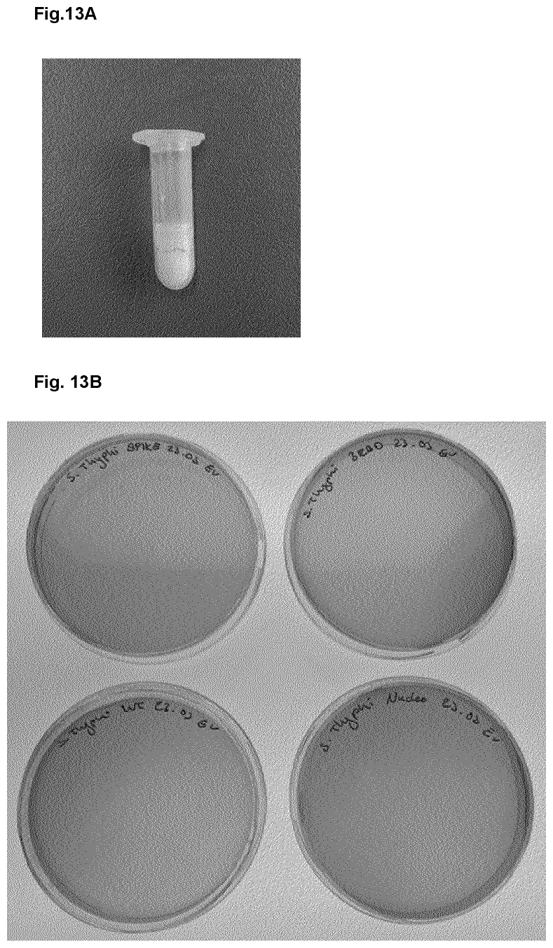

FIG. 13a shows a vial of suspended bacterium cells S. typhi Ty21a after lyophiliza-tion. The lyophilized cells were found to be non-viable when plated on LB agar plates as shown in FIG. 13b. Upper left: cells comprising pMATE Ara SP SARS-COV2 (spike protein); upper right: cells comprising pMATE Ara 3RBD (RBD with trimerization do-main); lower left: wild-type cells; lower right: cells comprising pMATE Ara NP SARS-CoV2 (nucleoprotein). No cell growth was visible overnight.

The plating procedure was performed as follows:
suspending the lyophilized cells (1 ml cell culture at an OD₅₇₈ of 10) in 1 ml ddH₂O;
plating 200 μl of the suspended lyophilized cells on an LB agar plate without antibiotics.

Lyophilized non-viable cells comprising pMATE Ara NP SARS-COV2 (nucleoprotein) and lyophilized S. typhi Ty21a wild-type cells were subjected to a SARS-COV-2 antigen test. The test with cells comprising the nucleoprotein gave a positive result.

The test procedure was performed as follows:
suspending the lyophilized cells (1 ml cell culture at an OD₅₇₈ of 10) in 1 ml ddH₂O;
applying 4 drops of the suspension to a test.

The immunogenicity was retained even after extended storage over extended time periods at 25° C. (at present up to 6 months or up to 12 months) and at 37° C. (at present up to 2 months or up to 12 months).

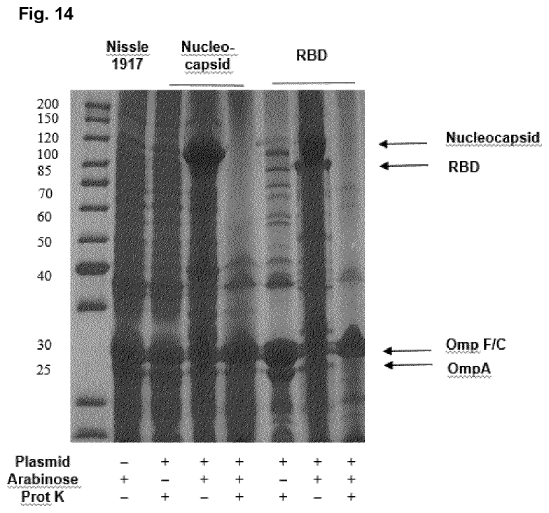

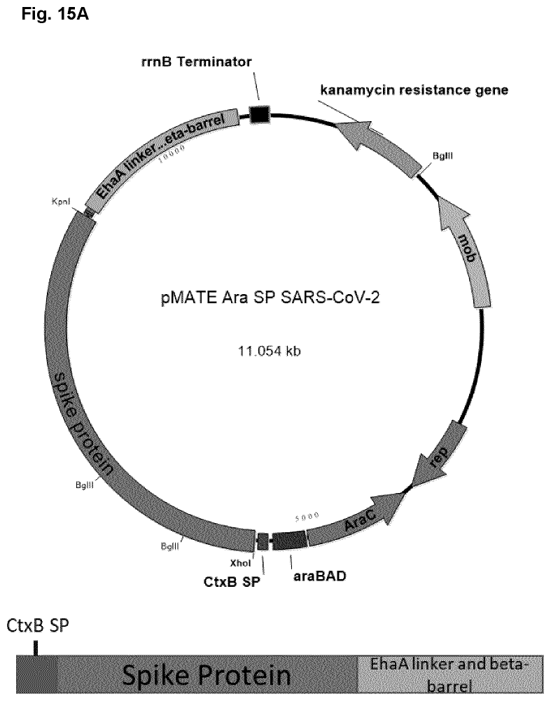

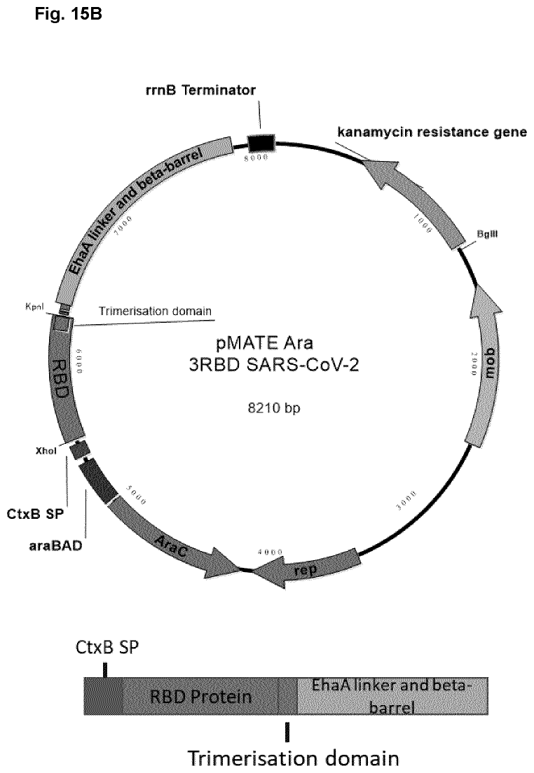

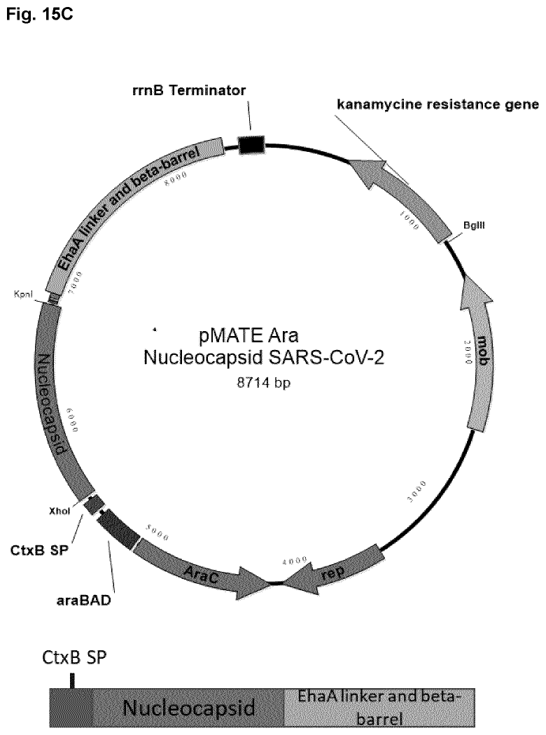

FIG. 14 shows the expression and surface display of SARS COV-2 nucleocapsid protein and the SARS COV-2 RBD in the vaccine strain E. coli Nissle 1917 in a Coomassie gel. Arabinose was added to induce expression of the fusion protein. Proteinase K was added to digest surface-exposed proteins.

A SARS COV-2 antigen test with E. coli Nissle 1917 displaying the nucleoprotein cells gave a positive result (not shown).

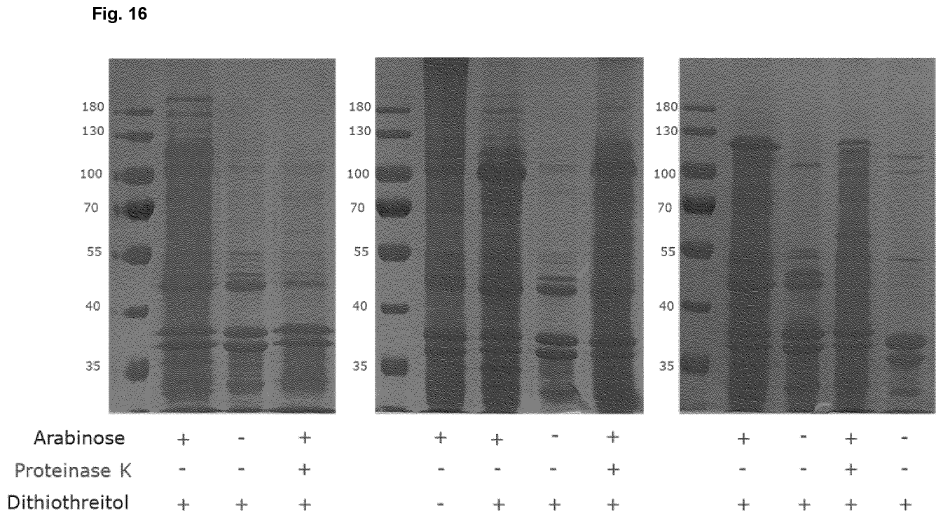

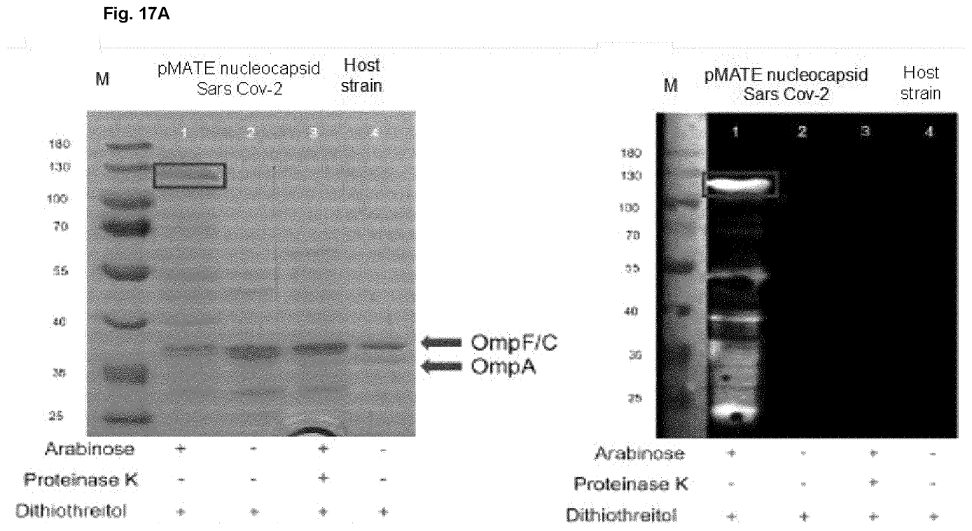

According to the methods described in WO 2014/139862, pMATE expression plas-mids were constructed. FIG. 15A shows the plasmid of pMATE Ara Spike protein SARS COV-2 (Spike(S) glycoprotein) and the corresponding proteins. FIG. 15B shows the plasmid of pMATE Ara 3RBD SARS COV-2 (receptor binding domain with N-terminal trimerization domain) and the corresponding proteins. FIG. 15C shows the plasmid of pMATE Ara nucleocapsid protein SARS COV-2 (nucleocapsid pro-tein) and the corresponding proteins.

FIG. 16 shows the expression and surface display of SARS COV-2 Spike protein (a), 3RBD with trimerization domain (b) and nucleocapsid (c) in S. typhi Ty 21a in a Coomassie gel in the presence or absence of dithiothreitol. Arabinose was added to induce expression of the fusion protein. Proteinase K was added to digest sur-face-exposed proteins.

The molecular weights of the proteins are as follows:
Spike-EhaA: 191.4 kDa
3RDD-EhaA: 82.8 kDa
Nucleocapsid-EhaA: 103.2 kDa

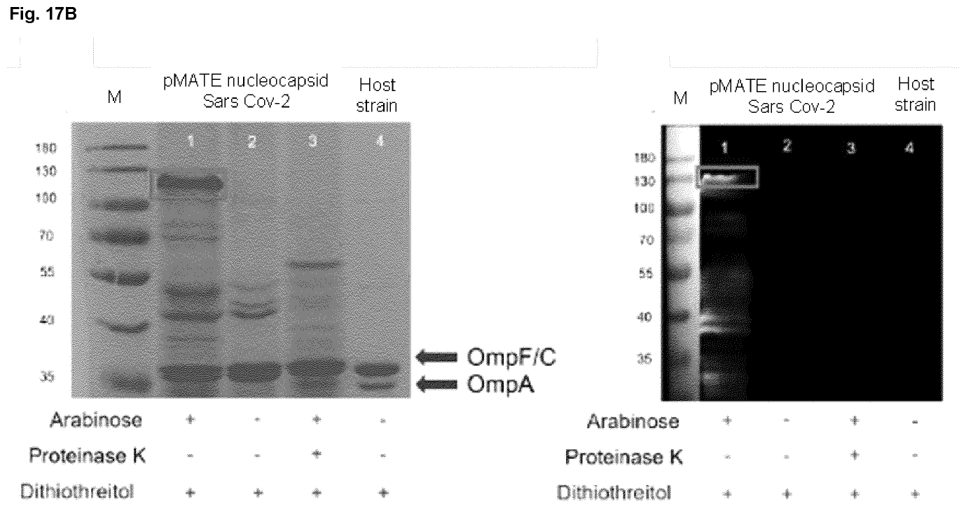

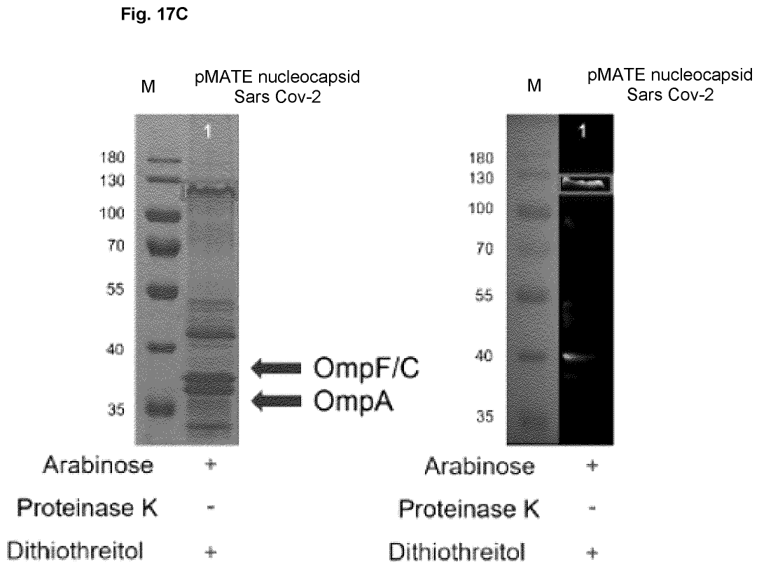

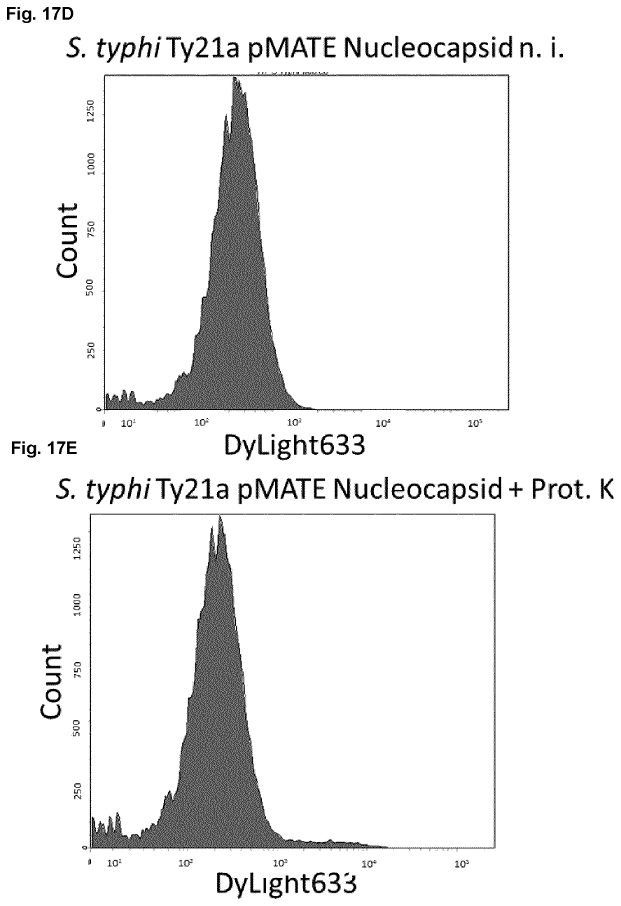

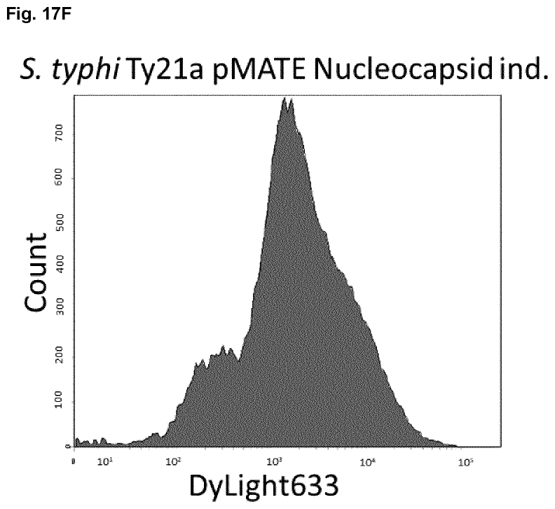

FIG. 17A-C show the expression and surface display of SARS COV-2 nucleocapsid protein in S. typhi Ty21a in a Coomassie gel (left) and a Western blot (right) in the presence of dithiothreitol. The nucleocapsid protein is depicted in the box of lane 1. Arabinose was added to induce expression of the fusion protein. Proteinase K was added to digest surface-exposed proteins. FIG. 17D-E show the amount of induced (ind.) and not induced (n.i.) nucleocapsid protein and proteinase K in S. typhi Ty 21a, measured via FACS.

The following results were obtained:
S. typhi Ty21a pMATE Nucleocapsid n. i.: Mean DyLight 633:261
S. typhi Ty21a pMATE Nucleocapsid ind.: Mean DyLight 633:3741
S. typhi Ty21a pMATE Nucleocapsid+Prot. K: Mean DyLight 633:389

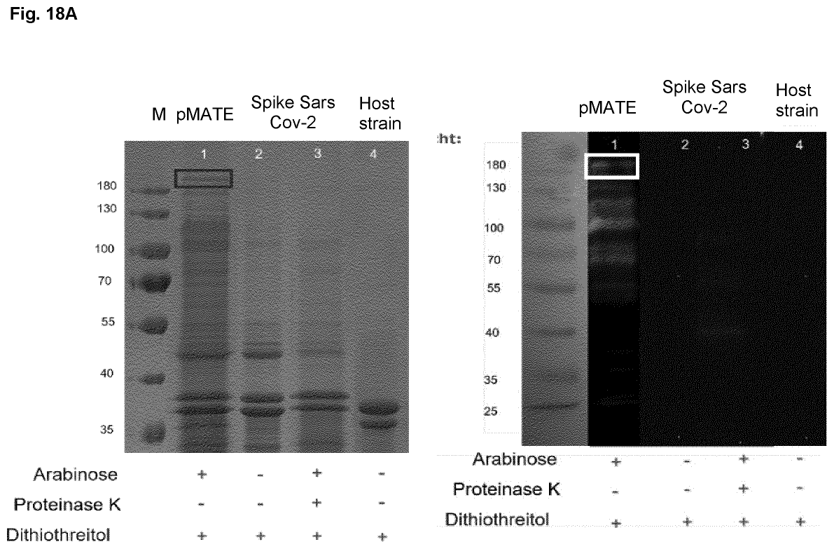

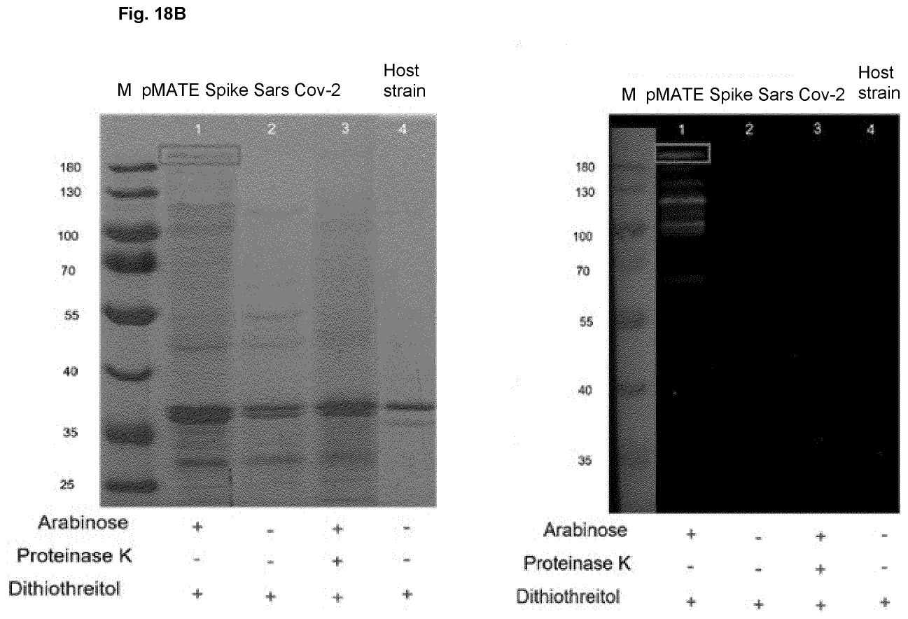

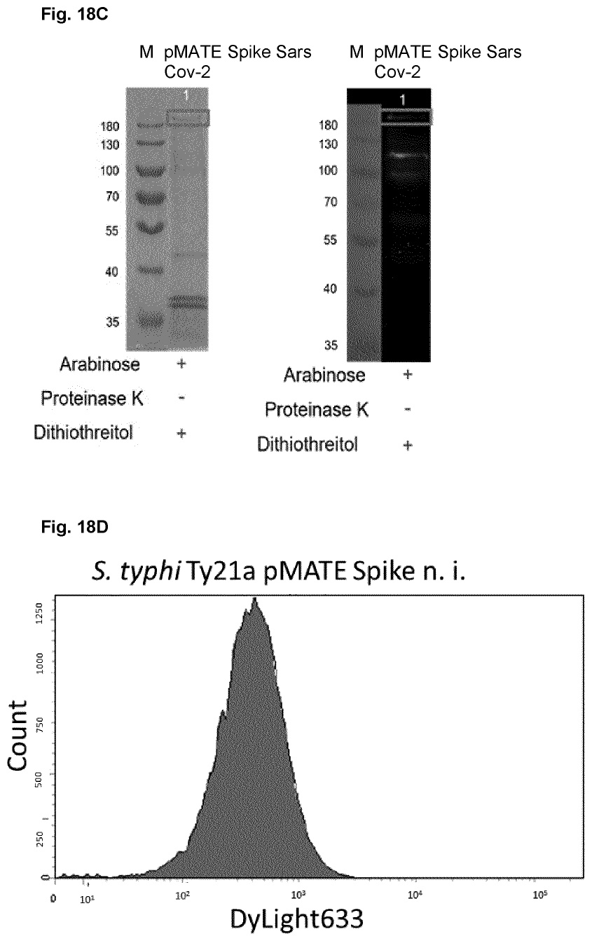

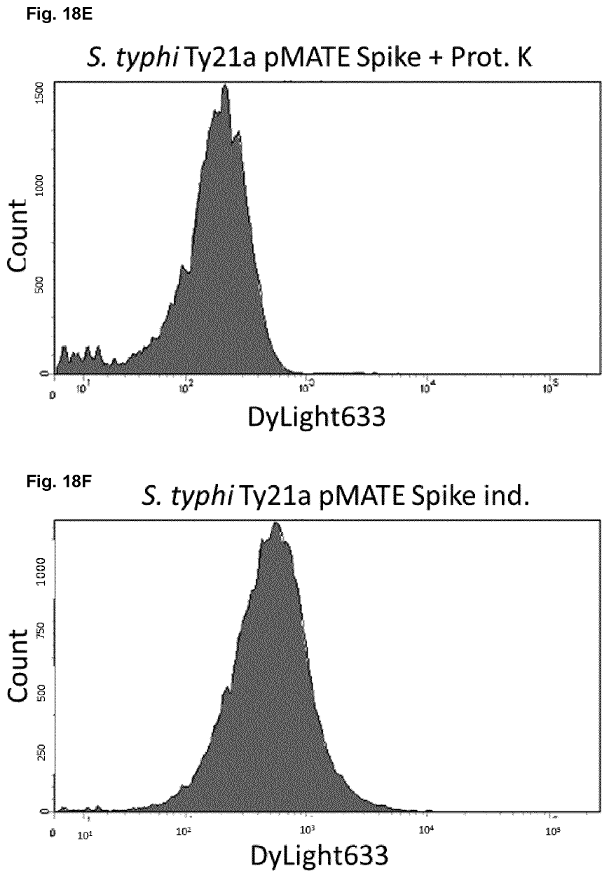

FIG. 18A-C show the expression and surface display of SARS COV-2 spike protein in S. typhi Ty21a in a Coomassie gel (left) and a Western blot (right) in the presence of dithiothreitol. The spike protein is depicted in the box of lane 1. Arabinose was added to induce expression of the fusion protein. Proteinase K was added to digest surface-exposed proteins. FIG. 18D-F show the amount of induced (ind.) and not induced (n.i.) spike protein and spike protein+proteinase K in S. typhi Ty 21a, measured via FACS.

The following results were obtained:
S. typhi Ty21a pMATE Spike n. i.: Mean DyLight 633:462
S. typhi Ty21a pMATE Spike ind.: Mean DyLight 633:759
S. typhi Ty21a pMATE Spike+Prot. K: Mean DyLight 633:229.

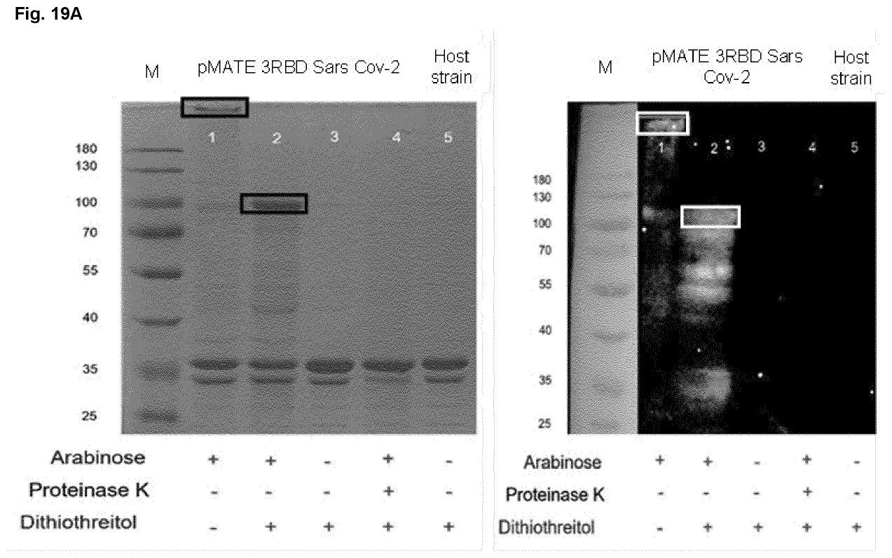

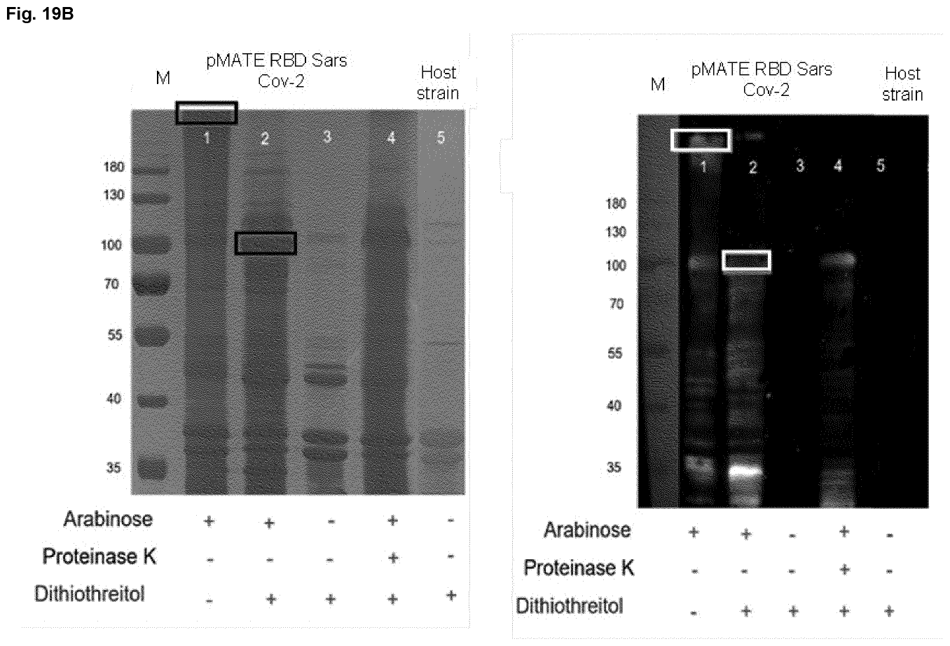

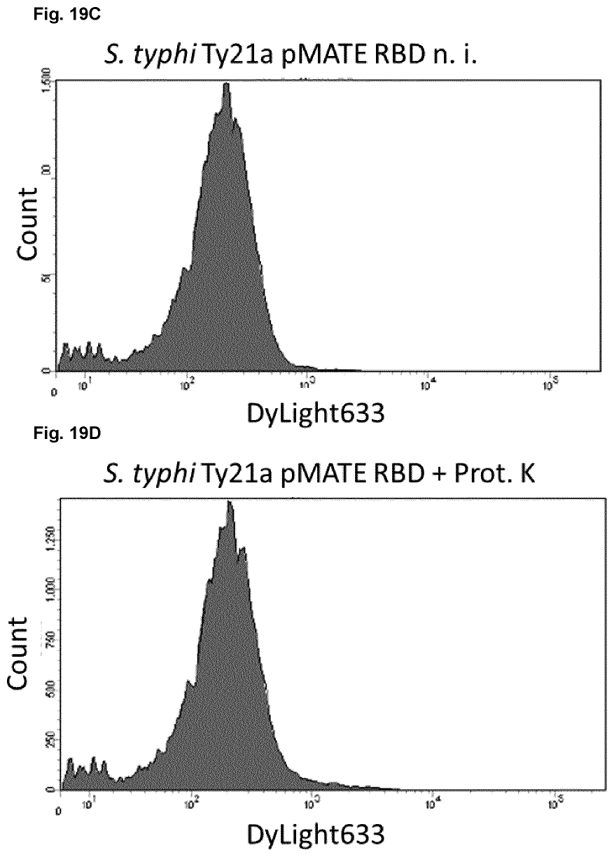

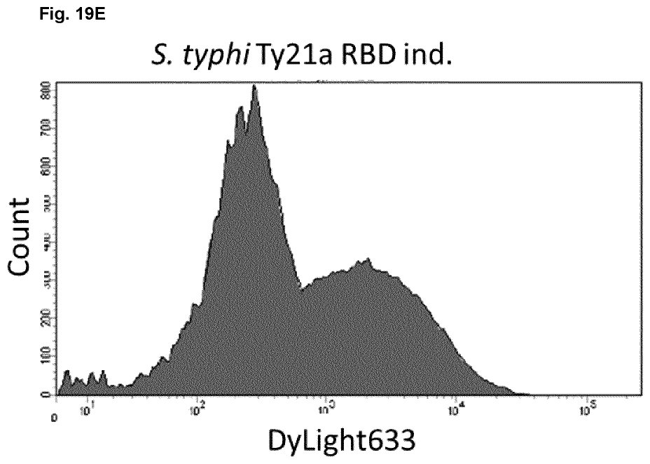

FIG. 19A shows the expression and surface display of SARS COV-2 3RBD with tri-merization domain in S. typhi Ty 21a in a Coomassie gel (left) and a Western blot (right). The trimeric RBD (w/o dithiothreitol) is depicted in the box of lane 1. The monomeric RBD (with dithiothreitol) is depicted in the box of lane 2. Arabinose was added to induce expression of the fusion protein. Proteinase K was added to digest surface-exposed proteins. FIG. 19B shows the expression and surface display of SARS COV-2 RBD with trimerization domain in S. typhi Ty 21a in a Coomassie gel (left) and a Western blot (right). The trimeric RBD (w/o dithiothreitol) is depicted in the box of lane 1. The monomeric RBD (with dithiothreitol) is depicted in the box of lane 2. Arabinose was added to induce expression of the fusion protein. Proteinase K was added to digest surface-exposed proteins. FIG. 19C-E show the amount of induced (ind.) and not induced (n.i.) RBD protein and RBD+proteinase K in S. typhi Ty 21a, measured via FACS.

The following results were obtained:
S. typhi Ty21a pMATE RBD n. i.: Mean DyLight 633:248
S. typhi Ty21a pMATE RBD ind.: Mean DyLight 633:1577
S. typhi Ty21a pMATE RBD+Prot. K: Mean DyLight 633:268

The S. typhi cells expressing the recombinant fusion proteins were harvested and lyophilized.

The lyophilization procedure was as described above.

FIG. 20A and FIG. 20B show a vial/scale pan of suspended bacterium cells S. typhi Ty21a after lyophilization. The lyophilized cells were found to be non-viable when plated on LB agar plates as shown in FIG. 20C. FIG. 20C: Upper left: cells comprising pMATE Ara SP SARS-COV2 (spike protein); upper right: cells comprising pMATE Ara 3RBD (RBD with trimerization domain); lower left: wild-type cells; lower right: cells comprising pMATE Ara NP SARS-COV2 (nucleoprotein). No cell growth was visible overnight.

The plating procedure was performed as follows:
suspending the lyophilized cells (1 ml cell culture at an OD₅₇₈ of 10) in 1 ml ddH₂O;
plating 200 μl of the suspended lyophilized cells on an LB agar plate without antibiotics.

Lyophilized non-viable cells comprising pMATE Ara NP SARS-COV2 (nucleoprotein) and lyophilized S. typhi Ty21a wild-type cells were subjected to a SARS-COV-2 antigen test. The test with cells comprising the nucleoprotein gave a positive result.

The test procedure was performed as follows:
suspending the lyophilized cells (1 ml cell culture at an OD₅₇₈ of 10) in 1 ml ddH₂O;
applying 4 drops of the suspension to a test.

The immunogenicity was retained even after extended storage over extended time periods at 25° C. (at present up to 6 months) and at 37° C. (at present up to 2 months).

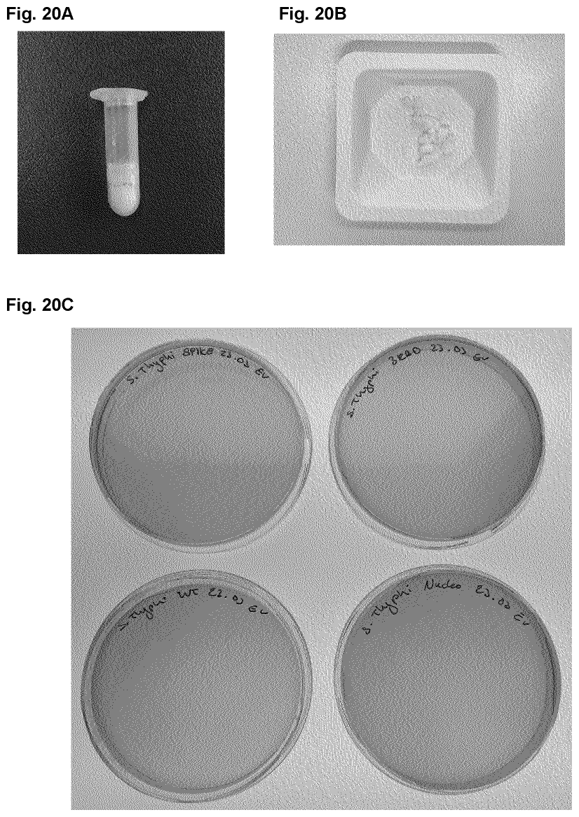

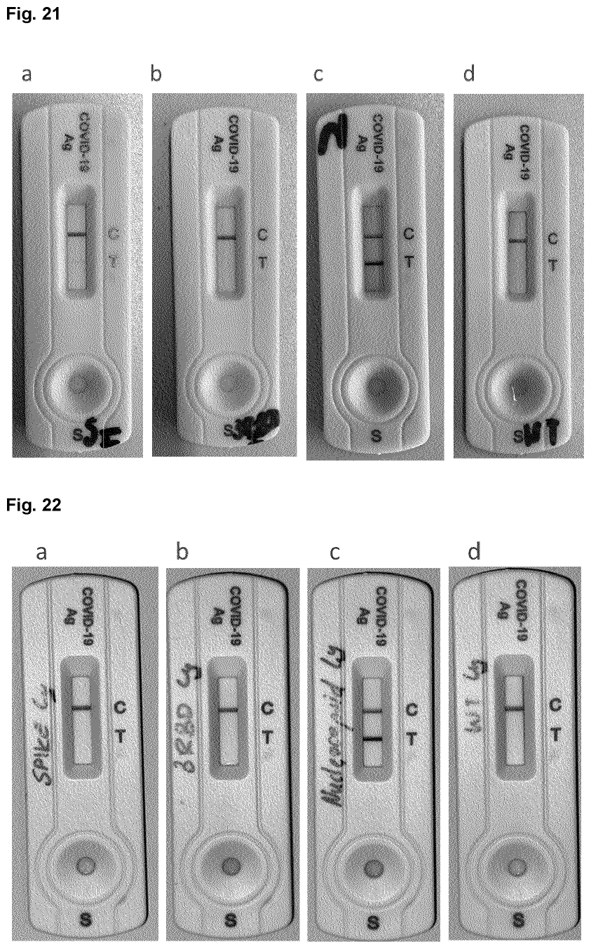

FIG. 21 shows Covid-19 antigen tests with the following Ty21a vaccine strains: S. typhi pMATE_{Ara}-spike protein (a), S. typhi pMATE_{Ara}-3RBD (b), S. typhi pMATE_{Ara}-nucleocapsid (c), and S. typhi (d). The test procedure was as follows:
Setting the cells to an OD₅₇₈ of 1 in PBS
4 drops of the suspension were applied to the antigen tests.

FIG. 22 shows Covid-19 antigen tests with the following lyophilized Ty21a vaccine strains: lyophilized S. typhi pMATE_{Ara}-spike protein (a), lyophilized S. typhi pMATE_{Ara}-3RBD (b), lyophilized S. typhi pMATE_{Ara}-nucleocapsid (c), and lyophilized S. typhi (d). The test procedure was as follows:
suspending the lyophilized cells (1 ml cell culture at an OD₅₇₈ of 10) in 1 ml ddH₂O;
4 drops of the suspension were applied to the antigen tests.

FIG. 23A shows Covid-19 antigen tests with lyophilized Ty21a vaccine strains 1 day after harvesting, while FIG. 23B shows Covid-19 antigen tests with Ty21a vaccine strains 2 days after lyophilization: nucleocapsid (a), spike protein (b), and RBD (c). The test procedure was as follows:
suspending the lyophilized cells (1 ml cell culture at an OD₅₇₈ of 10) in 1 ml ddH₂O;
4 drops of the suspension were applied to the antigen tests.

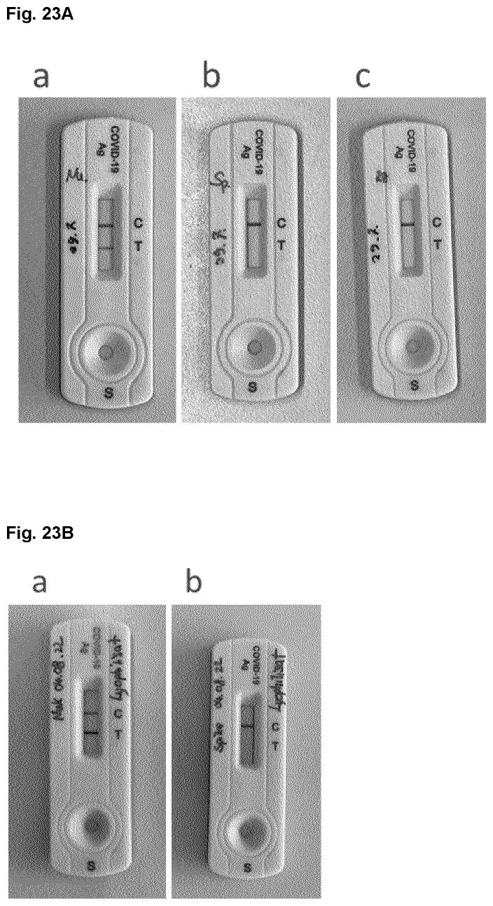

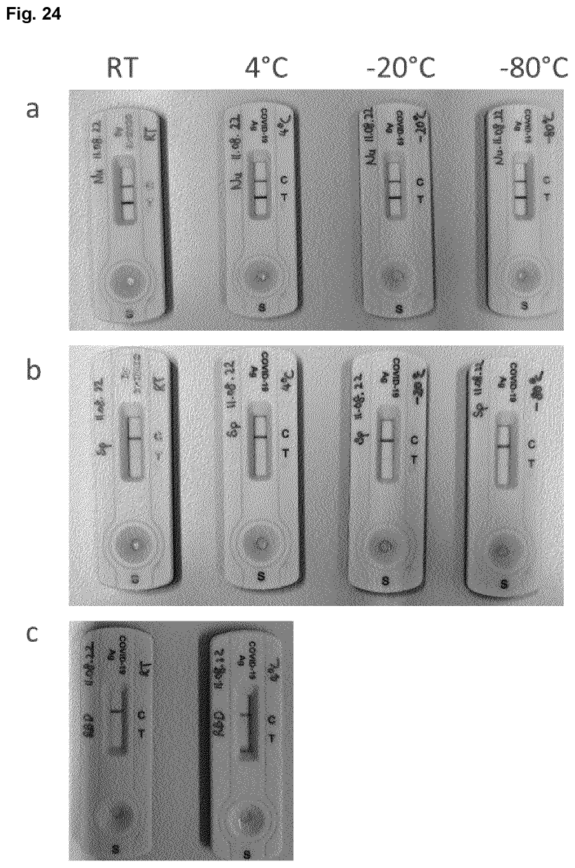

FIG. 24 shows Covid-19 antigen tests with the following lyophilized Ty21a vaccine strains after 1 week: nucleocapsid (a), spike protein (b), and RBD (c). Tests were conducted at room temperature (RT), 4° C., −20° C., and −80° C. The test procedure was as follows:
suspending the lyophilized cells (1 ml cell culture at an OD₅₇₈ of 10) in 1 ml ddH₂O;
4 drops of the suspension were applied to the antigen tests.

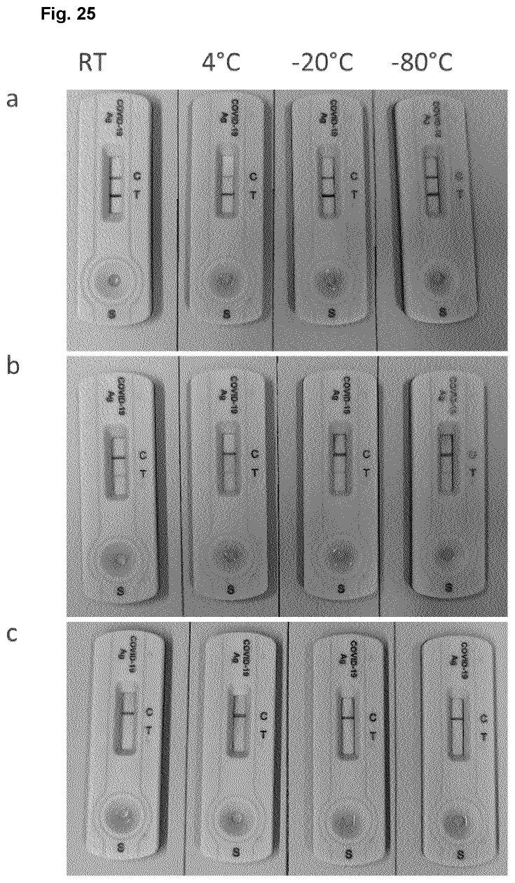

FIG. 25 shows Covid-19 antigen tests with the following lyophilized Ty21a vaccine strains after 4 weeks: nucleocapsid (a), spike protein (b), and RBD (c). Tests were conducted at room temperature (RT), 4° C., −20° C., and −80° C. The test procedure was as follows:
suspending the lyophilized cells (1 ml cell culture at an OD₅₇₈ of 10) in 1 ml ddH₂O;
4 drops of the suspension were applied to the antigen tests.

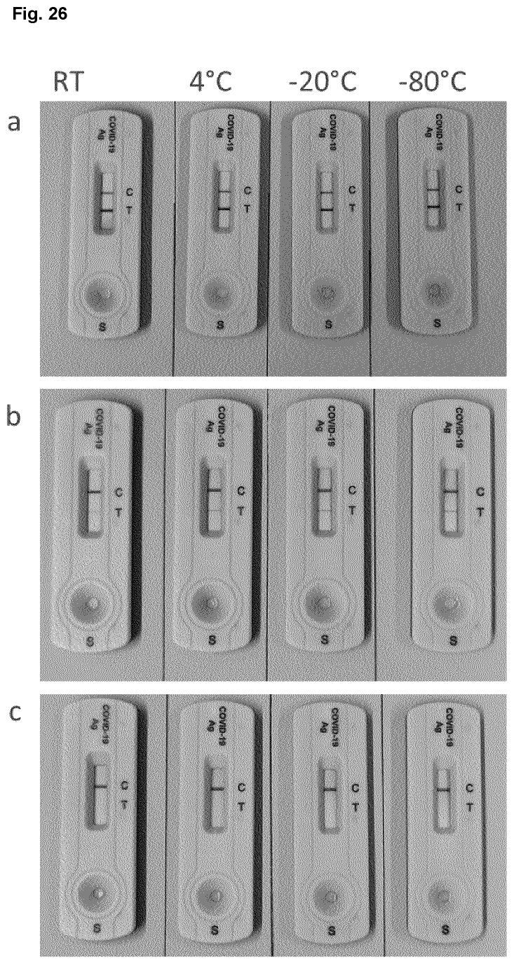

FIG. 26 shows Covid-19 antigen tests with the following lyophilized Ty21a vaccine strains after 2 months: nucleocapsid (a), spike protein (b), and RBD (c). Tests were conducted at room temperature (RT), 4° C., −20° C., and −80° C. The test procedure was as follows:
suspending the lyophilized cells (1 ml cell culture at an OD₅₇₈ of 10) in 1 ml ddH₂O;
4 drops of the suspension were applied to the antigen tests.

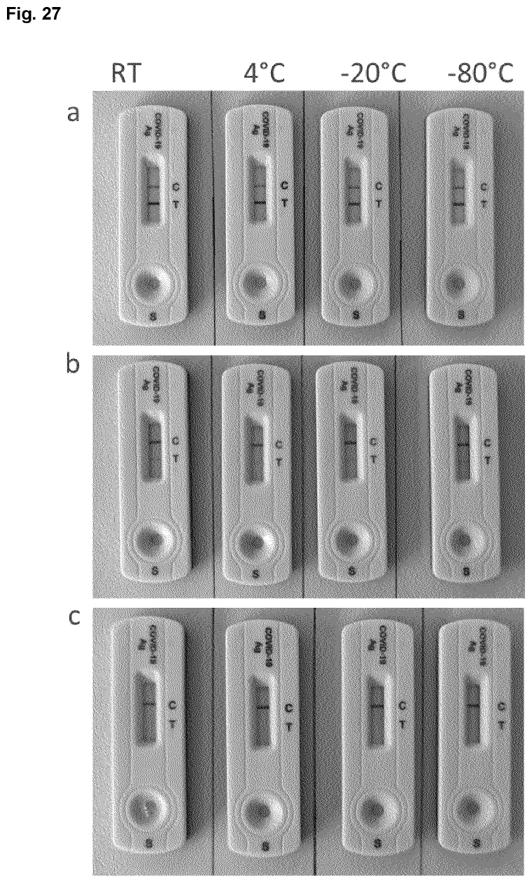

FIG. 27 shows Covid-19 antigen tests with the following lyophilized Ty21a vaccine strains after 3 months: nucleocapsid (a), spike protein (b), and RBD (c). Tests were conducted at room temperature (RT), 4° C., −20° C., and −80° C. The test procedure was as follows:
suspending the lyophilized cells (1 ml cell culture at an OD₅₇₈ of 10) in 1 ml ddH₂O;
4 drops of the suspension were applied to the antigen tests.

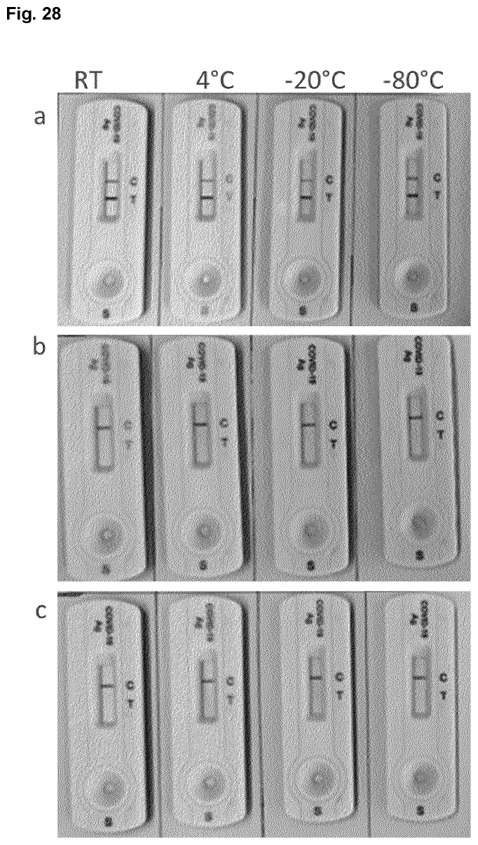

FIG. 28 shows Covid-19 antigen tests with the following lyophilized Ty21a vaccine strains after 12 months: nucleocapsid (a), spike protein (b), and RBD (c). Tests were conducted at room temperature (RT), 4° C., −20° C., and −80° C. The test procedure was as follows:
suspending the lyophilized cells (1 ml cell culture at an OD₅₇₈ of 10) in 1 ml ddH₂O;
4 drops of the suspension were applied to the antigen tests.

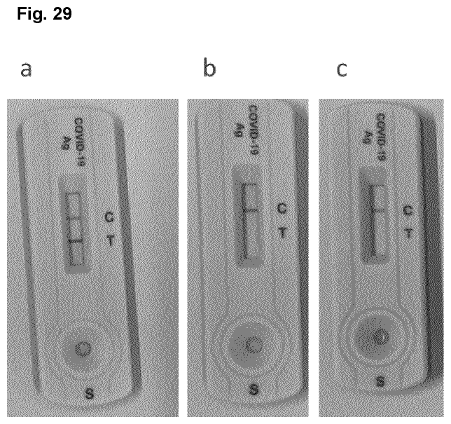

FIG. 29 shows Covid-19 antigen tests with the following lyophilized Ty21a vaccine strains after 12 months at 37° C.: nucleocapsid (a), spike protein (b), and RBD (c).

The test procedure was as follows:
suspending the lyophilized cells (1 ml cell culture at an OD₅₇₈ of 10) in 1 ml ddH₂O;
4 drops of the suspension were applied to the antigen tests.

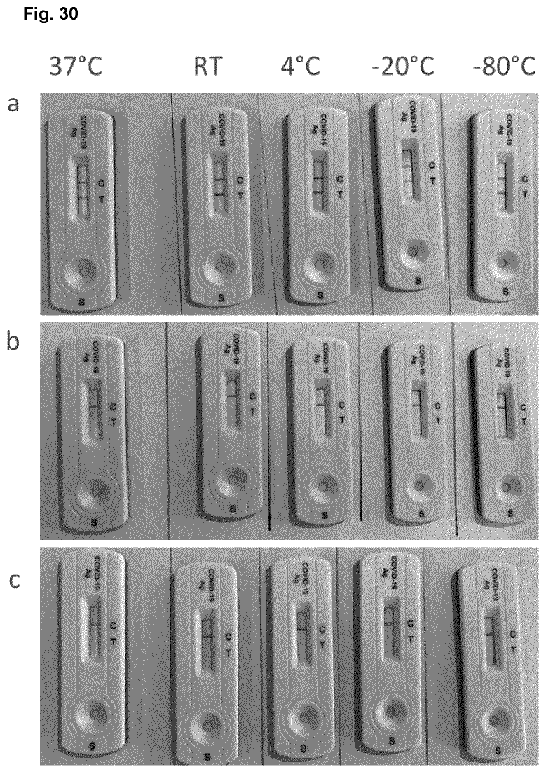

FIG. 30 shows Covid-19 antigen tests with the following lyophilized E. coli Nissle vaccine strains after 8 months: nucleocapsid (a), spike protein (b), and RBD (c). Tests were conducted at 37° C., room temperature (RT), 4° C., −20° C., and −80° C. The test procedure was as follows:
suspending the lyophilized cells (1 ml cell culture at an OD₅₇₈ of 10) in 1 ml ddH₂O;
4 drops of the suspension were applied to the antigen tests.

In sum, the storage (and transport) of the lyophilized preparation was without the need of a cool chain (at least for 12 month). A long-term storage of lyophilized prep-arations in both strains, Salmonella Ty21a and E. coli nissle, was observed without loss of antigenicity.

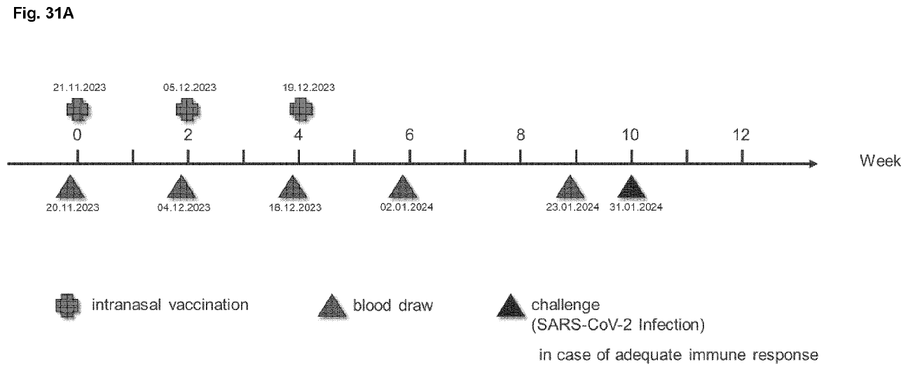

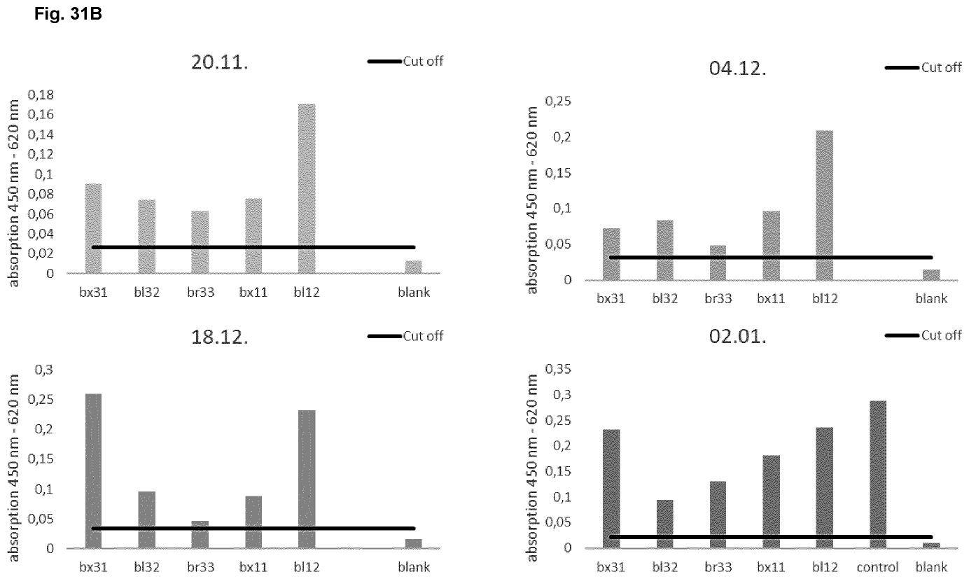

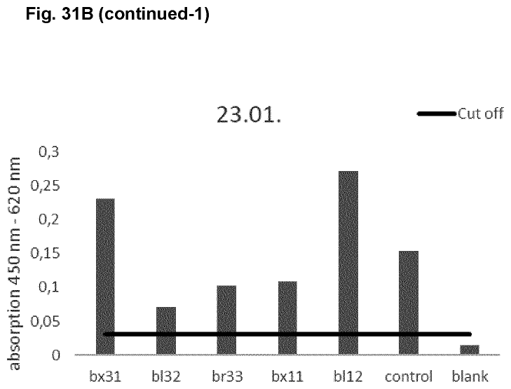

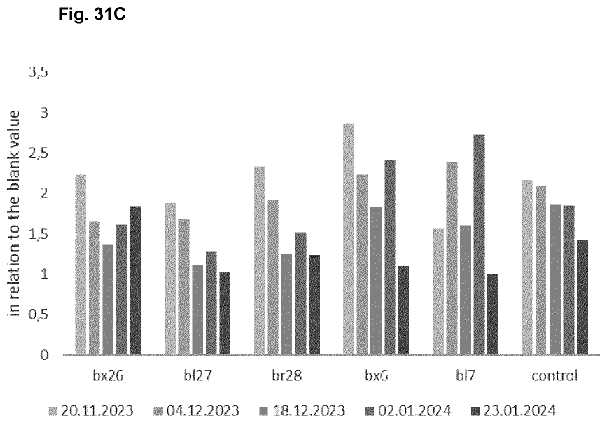

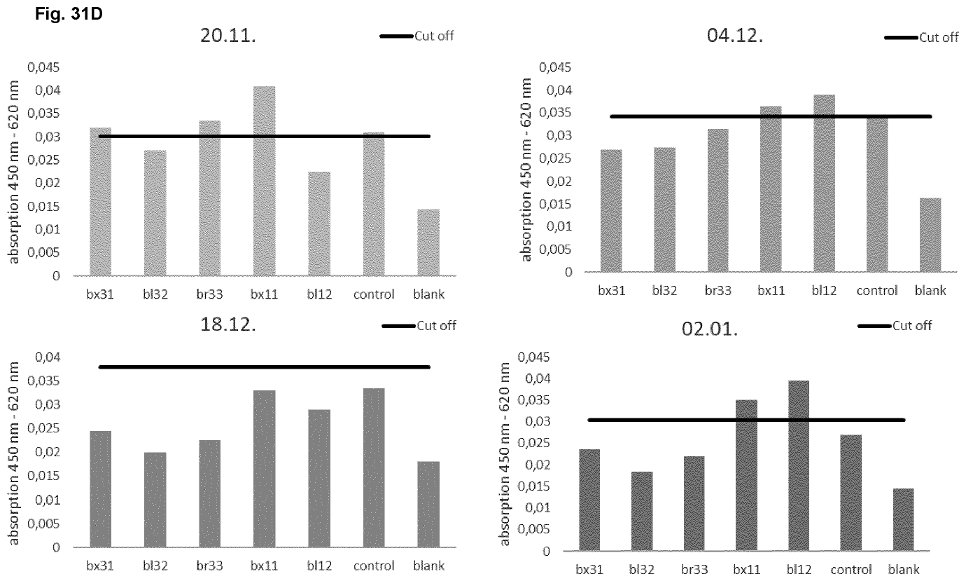

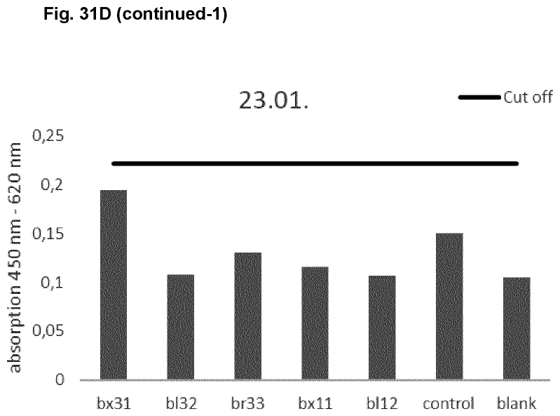

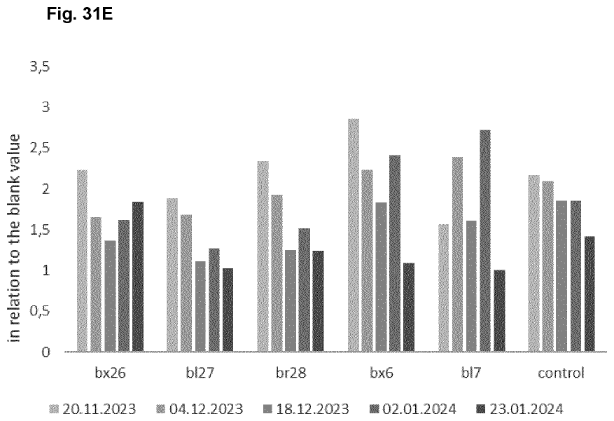

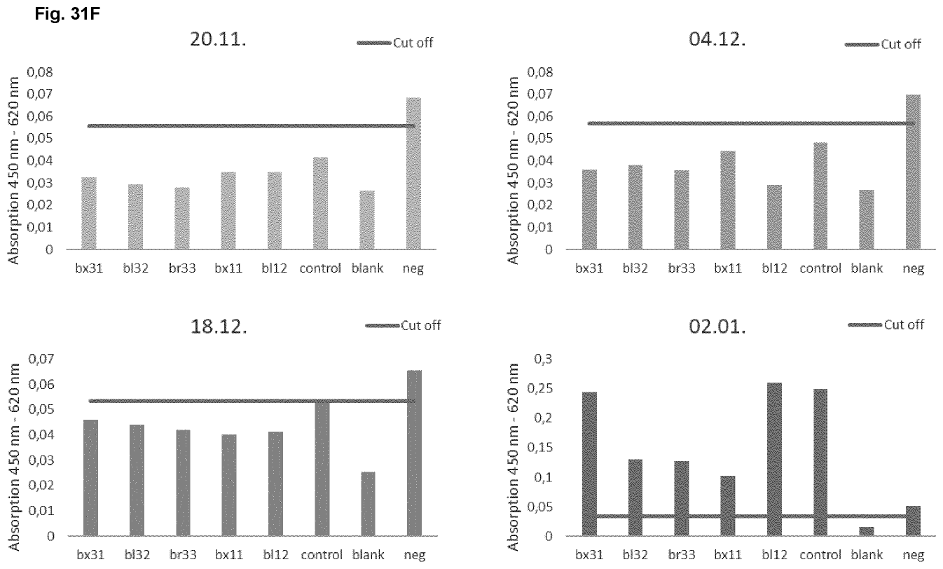

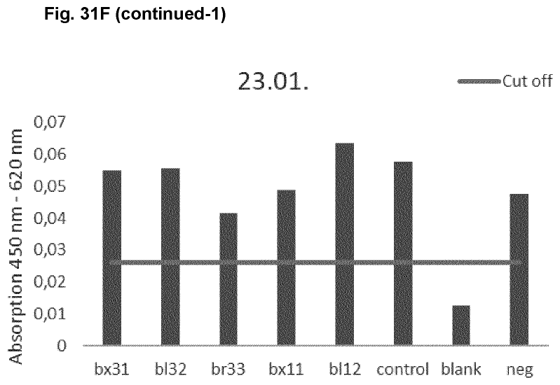

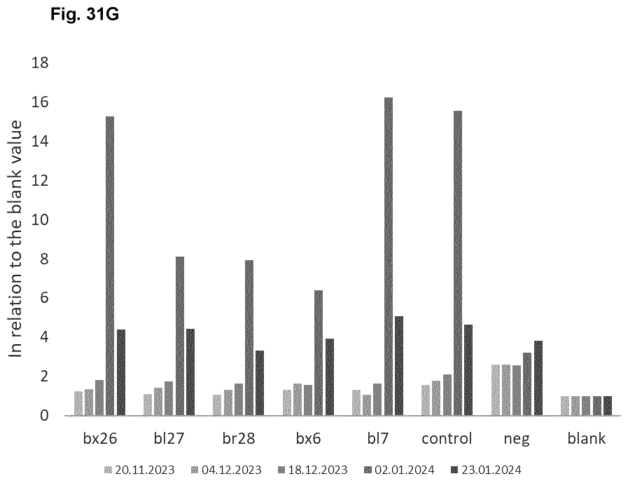

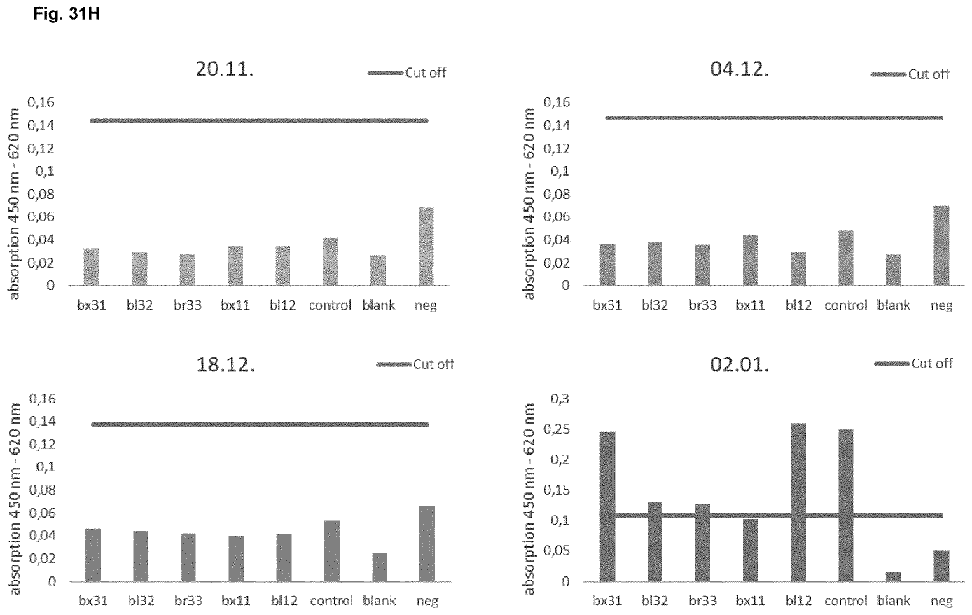

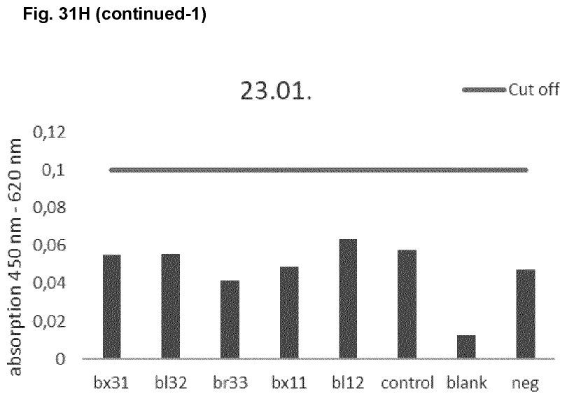

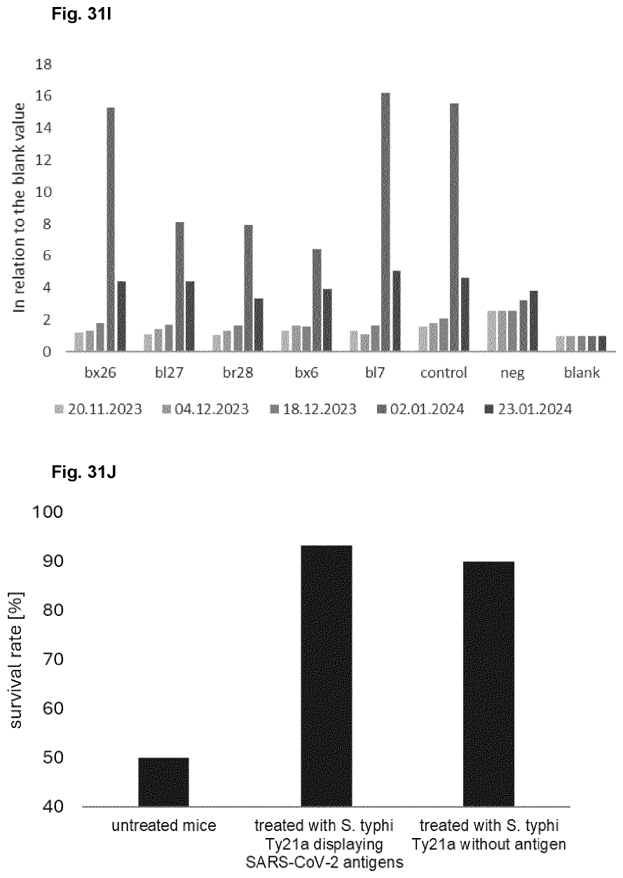

FIG. 31A shows the timeline of the immunization test procedure of BALB/c hACE2tg mice. On 21.11.2023, 05.12.2023 and 19.12.2023, intranasal vaccination was ap-plied to the mice. Before (20.11.2023) and after the intranasal vaccination (04.12.2023, 18.12.2023; 02.01.2024 and 23.01.2024) blood was taken and the ab-sorption analysed at 450 nm-620 nm. “Control” refers to cells without antigen; “Neg” refers to a negative control provided by the ELISA manufacturer. FIG. 31B and FIG. 31C show the nucleocapsid-specific IgG-response in the sera of mice treated with S. typhi nucleocapsid. FIG. 31D and FIG. 31E show the RBD-specific IgG-response in the sera of mice treated with S. typhi RBD. FIG. 31F and FIG. 31G show anti-spike protein IgG-response in the sera of mice treated with S. typhi spike protein. The ELISA spike protein is in relation to the blank. FIG. 31H and FIG. 311 show anti-nucleocapsid IgG-response in the sera of mice treated with S. typhi nu-cleocapsid. The ELISA spikeprotein is in relation to neg. FIG. 31J shows the survival rate in [%] of mice infected with a LD₅₀ of SARS-COV-2 after treatment with S. typhi vaccines. LD₅₀: 2.5×10³ SARS-COV-2 virus particles.

The immunization of BALB/c hACE2tg mice with spike protein, RBD and nucleocap-sid protein led to different IgG response. Application of the vaccine was possible as a sterile effervescent powder (no water required). The formulation as an efferves-cent powder is not erasing antigenicity.

FIG. 32 shows the development of an effervescent powder (ePowder). The left vial shows the effervescent powder without cells and water. In the right vial, water was added to the effervescent powder (without cells).

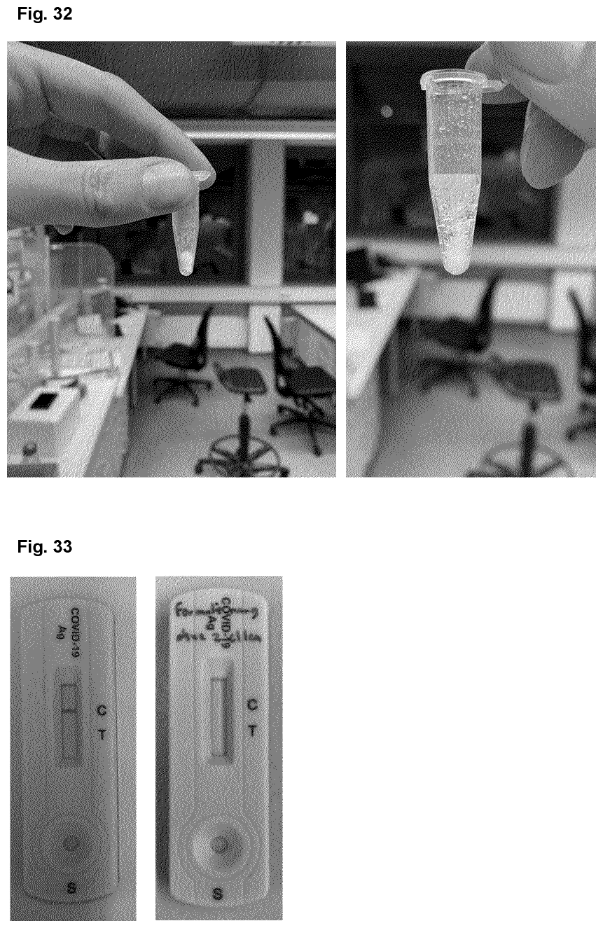

FIG. 33 shows Covid-19 antigen tests with ePowder (left; 0.05 g NaHCO₃, 0.05 g citric acid and 490 μL ddH₂O) or the formulation according to german pharmaco-poeia (NRF) 20.2 (right; 0.05 g NaHCO₃, 0.05 g citric acid, 0.091 g sorbitol, 0.83*10⁻³ g Syloid 244 FP, and 490 μL ddH₂O).

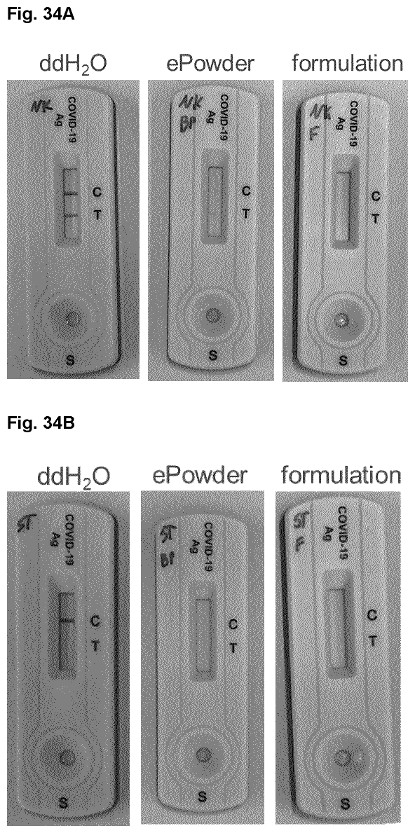

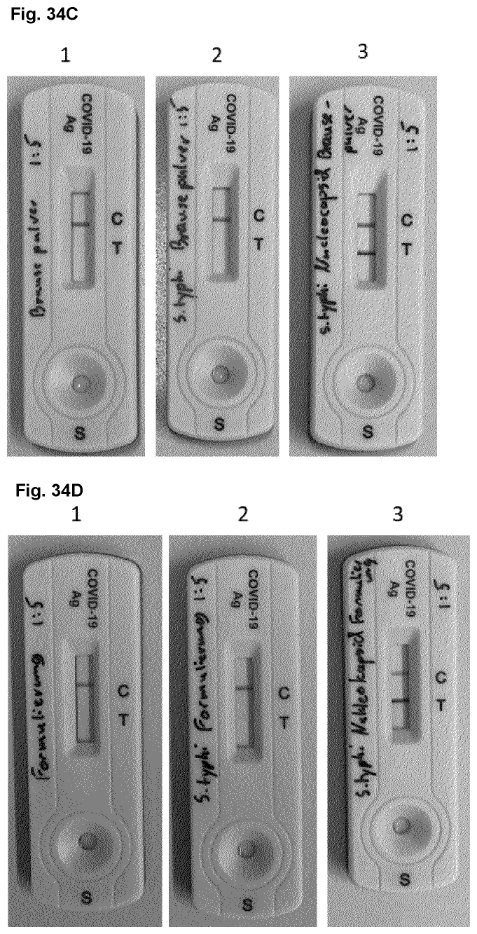

FIG. 34A shows Covid-19 antigen tests of S. typhi Ty21a pMATE nucleocapsid with ddH₂O, ePowder, or formulation. FIG. 34B shows Covid-19 antigen tests of S. typhi Ty21a with ddH₂O, ePowder, or formulation. FIG. 34C shows the comparison of Covid-19 antigen tests in case of ePowder: (1): ddH₂O; (2): S. typhi Ty21a; (3): S. typhi Ty21a with nucleocapsid. ePowder: 0.05 g NaHCO₃, 0.05 g citric acid, 490 μL ddH₂O and lyophilised cells (from 500 μL OD₁ in PBS). FIG. 34D shows the com-parison of Covid-19 antigen tests in case of the formulation: (1): ddH₂O; (2): S. typhi Ty21a; (3): S. typhi Ty21a with nucleocapsid. Formulation: 0.05 g NaHCO₃, 0.05 g citric acid, 0.091 g sorbitol, 0,83*10⁻³ g syloide 244 FP, 490 μL ddH₂O and lyophi-lised cells (from 500 μL OD₁ in PBS).

In case of FIG. 34C and FIG. 34D: Identical treatment as in the experiments before, but 1:5 diluted in ddH₂O right before the test No problems such as stucking of the dye in the antigen test were observed.

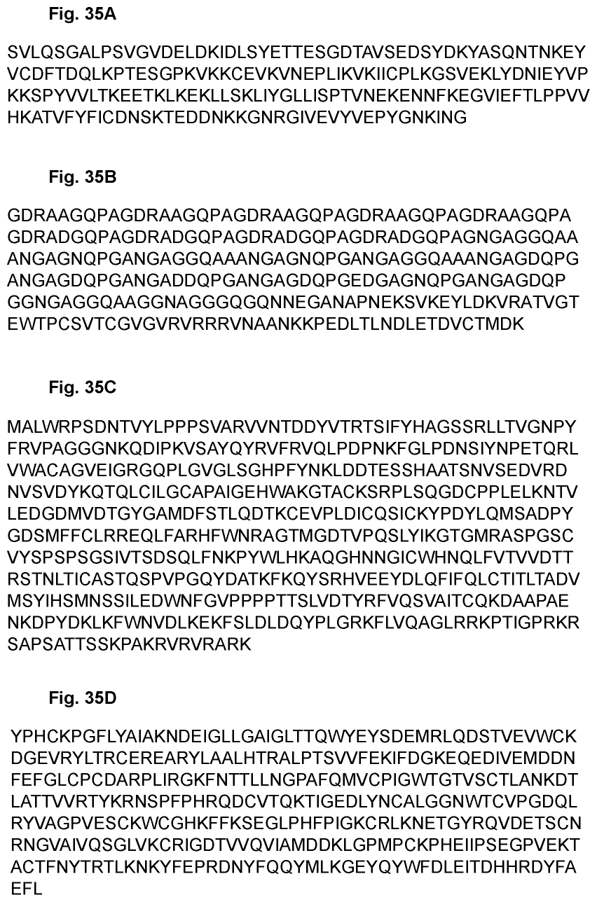

FIG. 35A shows the amino acid sequence of Pfs230D1M (corresponding to SEQ ID NO: 9)-a part of surface-associated 6-cystein domain of Plasmodium falciparum sexual stadium protein Pfs230 (Malaria). FIG. 35B shows the amino acid sequence of RV21 (corresponding to SEQ ID NO: 10)-a part of surface associated circum-sporozoit-protein, sporozoid stadium of Plasmodium falciparum (Malaria). FIG. 35C shows the amino acid sequence of the L1 protein of human papilloma virus (corre-sponding to SEQ ID NO: 11). FIG. 35D shows the amino acid sequence of N-termi-nal amino acids (22 kDa) of the BVDV E2 protein (BVDV344) (corresponding to SEQ ID NO: 12).

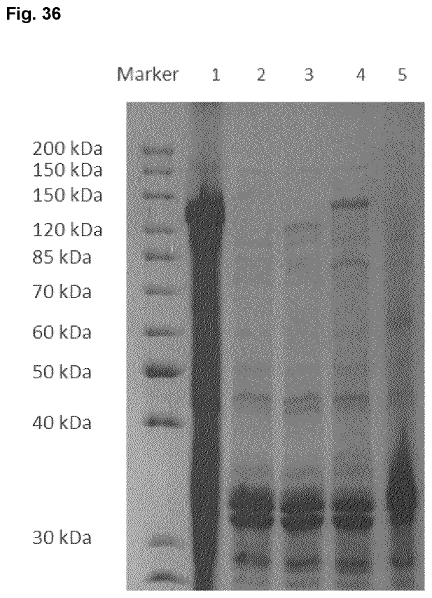

FIG. 36 shows the expression of four further antigens in E. coli (two malaria antigens, one HPV antigen and one BVD antigen).

The calculated sizes are as follows:
MATE-BVDV344: 99.96 kDa
MATE-HPVL1-18:117.15 kDa
MATE-Pfs230D1M: 82.47 kDa
MATE-RV21: 85.62 kDa

FIG. 36 demonstrates the autodisplay of two malaria antigens, one HPV antigen and one BVD antigen in E. coli.

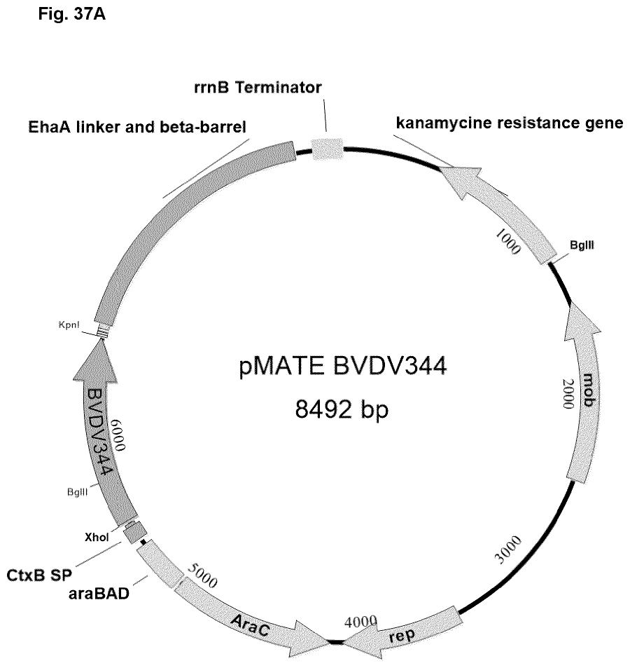

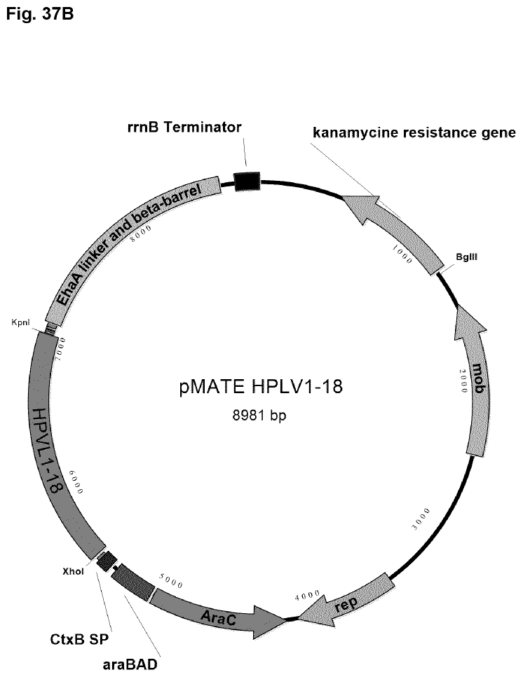

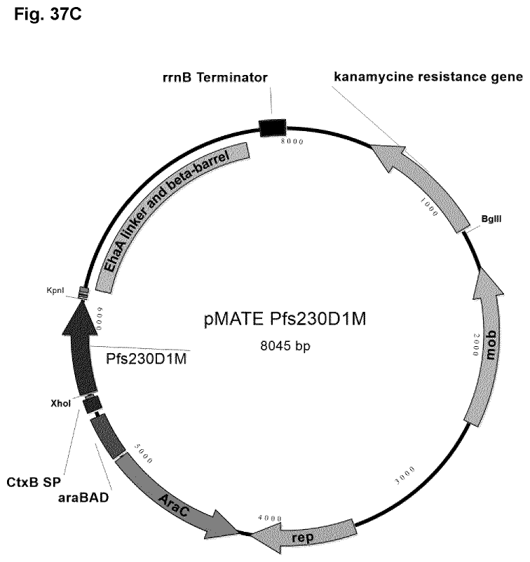

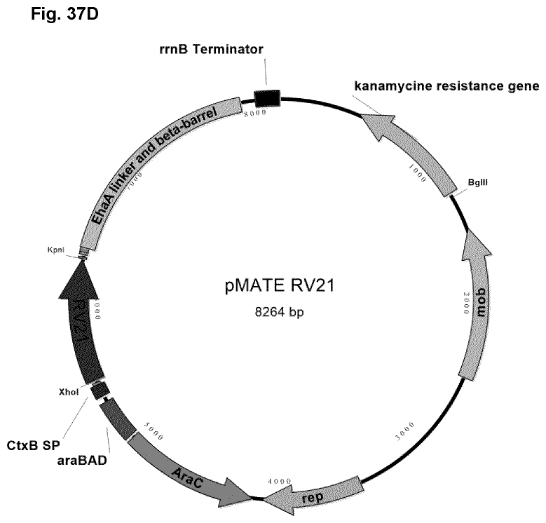

According to the methods described in WO 2014/139862, pMATE expression plas-mids were constructed. FIG. 37A shows the plasmid of pMATE BVDV344. FIG. 37B shows the plasmid of pMATE HPVL1 of HPV-type 18. FIG. 37C shows the plasmid of pMATE Pfs230D1M. FIG. 37D shows the plasmid of pMATE RV21.

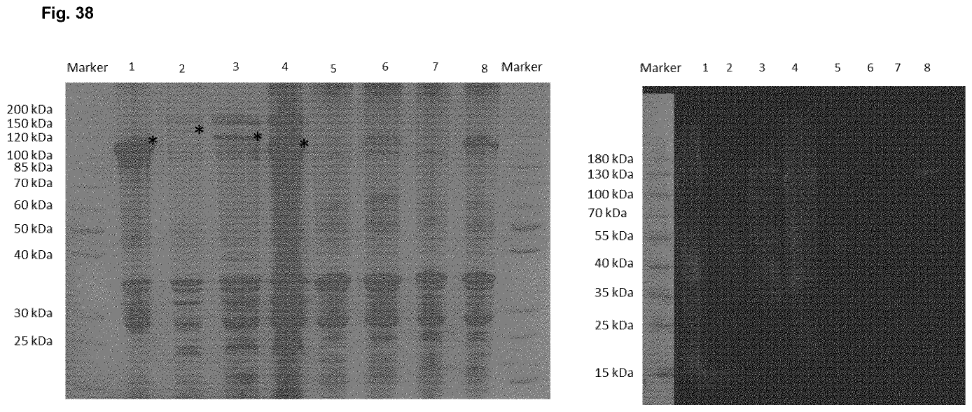

FIG. 38 shows the expression and surface display of BVDV, HPV and Malaria anti-gens in S. typhi Ty 21a in a Coomassie gel (left) and a Western blot (right). The numbers are as follows (S. typhi Ty21a pMATE):
1: BVDV344 ind.
2: HPVL1 of PHV-type 18 ind.
3: RV21 ind.
4: Pfs230D1M ind.
5: BVDV344 n. i.
6: HPVL1 of HPV-type 18 n.i.
7: RV21 n. i.
8: Pfs230D1M n. i.
(n. i. =not induced; ind. =induced)

The S. typhi cells expressing the recombinant fusion proteins may be harvested and lyophilized aas described above.

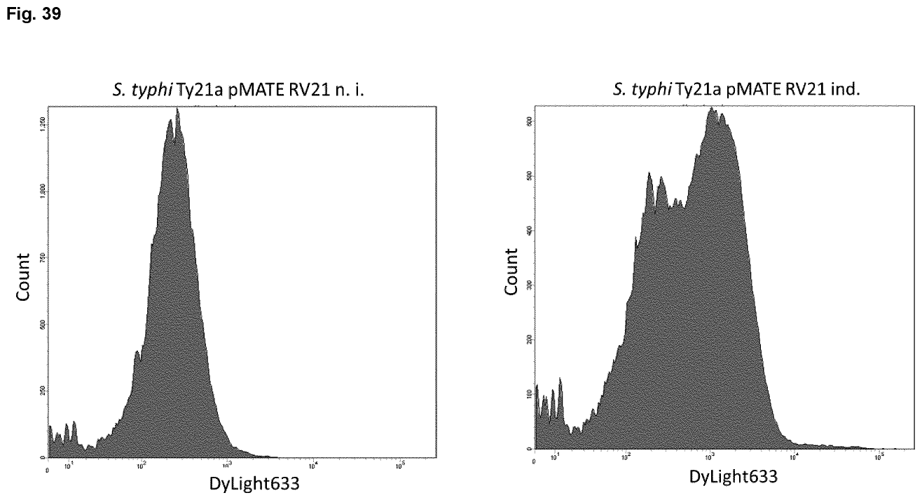

FIG. 39 shows the proof of surface display for S. typhi Ty21a pMATE RV21. Left: S. typhi Ty21a pMATE RV21 n. i.; right: S. typhi Ty21a pMATE RV21 ind. For the ex-periment, 50.000 cells were marked, treated with primary mouse anti 6xHis and secondary goat anti mouse DyLight 633 antibody and measured with a BD FACSAria™ III flow cytometer. For S. typhi Ty21a pMATE RV21 n. i. the mean DyLight 633 value was 297 and for S. typhi Ty21a pMATE RV21 ind, the mean DyLight 633 value was 1254 (n. i. =not induced; ind. =induced).

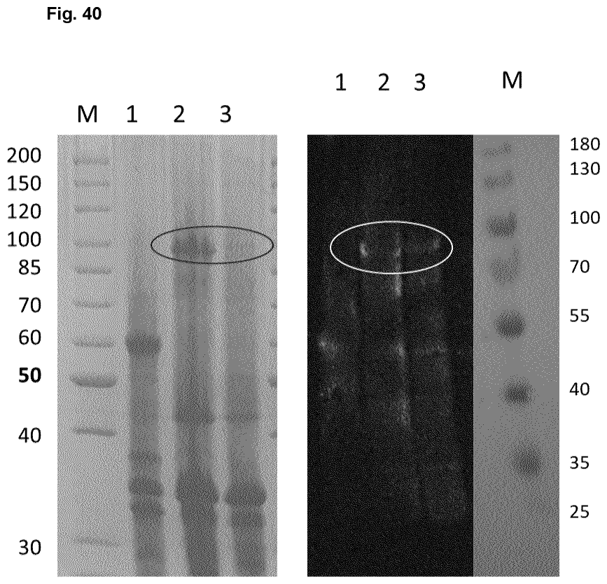

FIG. 40 shows the expression and surface display for E. coli Nissle pMATE BVDV344 in a Coomassie gel (left) and a Western blot (right). The numbers are as follows (E. coli Nissle):
1—pMATE without passenger
2—pMATE BVDV344
3—pMATE BVDV344+Prot. K

1 (pMATE without passenger) has the CtxB signal peptide, 6xHis, the linker and the β-barrel, representing the “empty pMATE vector” that serves as control.

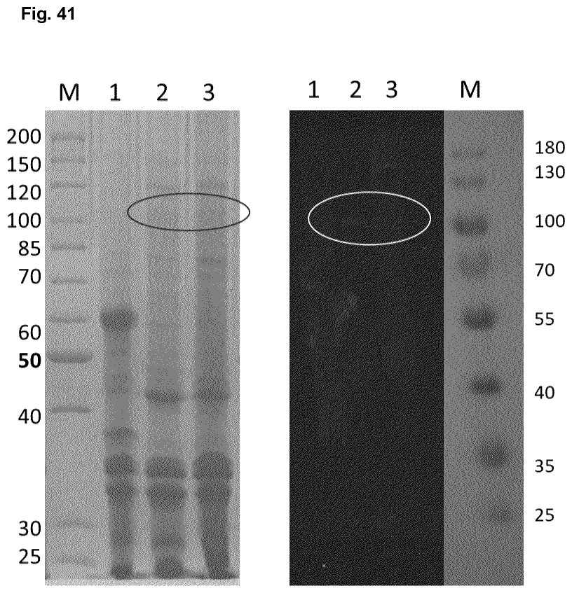

FIG. 41 shows the expression and surface display for E. coli Nissle pMATE HPVL of HPV-type 18 in a Coomassie gel (left) and a Western blot (right). The numbers are as follows (E. coli Nissle):
1—pMATE without passenger
2—pMATE HPVL1-18
3—pMATE HPVL1-18+Prot. K

1 (pMATE without passenger) has the CtxB signal peptide, 6xHis, the linker and the β-barrel, representing the “empty pMATE vector” that serves as control.

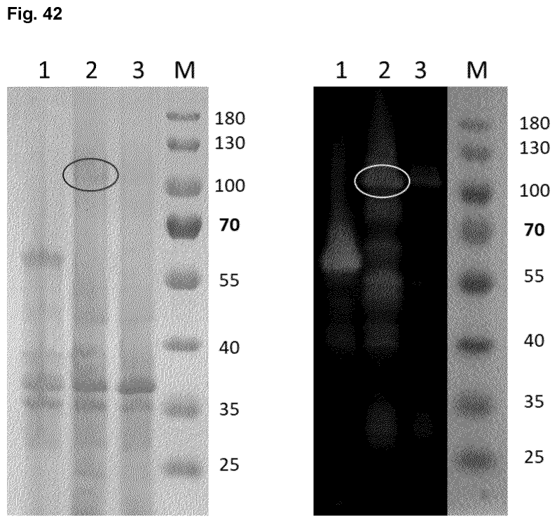

FIG. 42 shows the expression and surface display for E. coli Nissle pMATE Pfs230D1M in a Coomassie gel (left) and a Western blot (right). The numbers are as follows (E. coli Nissle):
1—pMATE without passenger
2—pMATE Pfs230D1M
3—pMATE Pfs230D1M+Prot K

1 (pMATE without passenger) has the CtxB signal peptide, 6xHis, the linker and the β-barrel, representing the “empty pMATE vector” that serves as control.

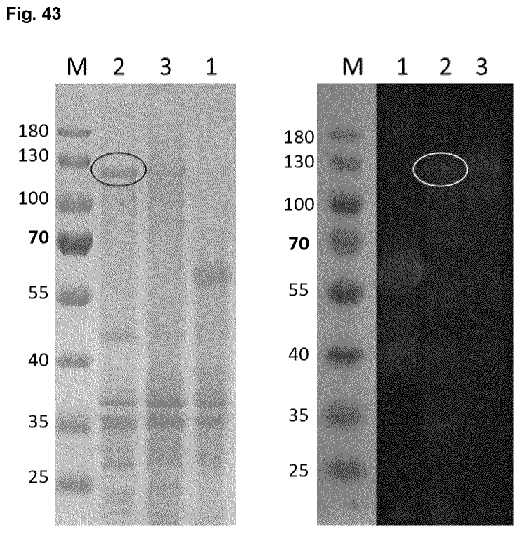

FIG. 43 shows the expression and surface display for E. coli Nissle pMATE RV21 in a Coomassie gel (left) and a Western blot (right). The numbers are as follows (E. coli Nissle):

1—pMATE without passenger
2—pMATE RV21
3—pMATE RV21+Prot. K

1 (pMATE without passenger) has the CtxB signal peptide, 6xHis, the linker and the β-barrel, representing the “empty pMATE vector” that serves as control.

1. A preparation comprising dried non-viable bacterium cells wherein an immunogenic polypeptide fused to an autotransporter comprising a transmembrane linker and a transporter domain is displayed on the surface of the cells.

2. The preparation of claim 1, wherein the non-viable bacterium cells are transformed or transfected with a recombinant nucleic acid molecule comprising:
(i) a portion encoding a signal peptide,
(ii) a portion encoding the immunogenic polypeptide to be displayed,
(iii) a portion encoding an autotransporter comprising a trans-membrane linker and a transporter domain, or
(i) a portion encoding a signal peptide,
(ii) a portion encoding an autotransporter comprising a transporter domain and a transmembrane linker; and
(iii) a portion encoding the immunogenic polypeptide to be dis-played.

3. The preparation of claim 1, wherein the autotransporter comprises the transporter domain of an EhaA protein or the autotransporter domain of a YeeJ protein.

4. The preparation of claim 1, wherein the bacterium cells are Gram-negative bacterium cells, e.g., Salmonella typhi Ty21a, E. coli Nissle 1917 or Vibrio cholerae Vaxchora.

5. The preparation of claim 1, wherein the immunogenic polypeptide is from a pathogen, e.g., a virus, bacterium, fungus, or parasite.

6. The preparation of claim 1, wherein the immunogenic polypeptide is from a coronavirus, e.g., MERS, SARS-COV-1, or SARS-COV-2, or from a Plasmodium organism, e.g., Plasmodium falciparum, or from a human papillomavirus, or from a bovine viral diarrhea virus.

7. The preparation of claim 1, wherein the immunogenic polypeptide is a SARS-COV-2 spike glycoprotein or an immunogenic fragment thereof, a SARS-COV-2 receptor binding domain or an immunogenic fragment thereof optionally including a trimerization domain, or a SARS-COV-2 nucleocapsid protein or an immunogenic fragment thereof.

8. The preparation of claim 1, wherein the immunogenic polypeptide is a surface-associated 6-cysteine domain of Plasmodium falciparum sexual stadium protein Pfs230 or an immunogenic fragment thereof, in particular Pfs230D1M, a surface associated circumsporozoid-protein of Plasmodium falciparum or an immunogenic fragment thereof, in particular RV21, a L1 protein of a human papillomavirus or an immunogenic fragment thereof, in particular HPVL1 from HPV-type 18, a mixture of tumorigenic HPV-types, in particular a mixture of HPV-types 6, 11, 16, 18, 31, 33, 45, 52, and 58, or a bovine viral diarrhea virus E2 protein or an immunogenic fragment thereof, in particular BVDV344.

9. The preparation of claim 1, wherein the preparation is free from living bacterial cells.

10. The preparation of claim 1, wherein the bacterium cells are lyophilized.

11. The preparation of claim 1, further comprising phosphate ions and which optionally further comprises sodium, potassium and/or chloride ions.

12. The preparation of claim 1, wherein the non-viable bacterium cells comprise a first immunogenic polypeptide and a second immunogenic polypeptide, wherein the first immunogenic polypeptide is different from the second immunogenic polypeptide.

13. The preparation of claim 12, wherein the first immunogenic polypeptide and the second immunogenic polypeptide are from the same pathogen or are from different pathogens.

14. The preparation of claim 12, wherein the first immunogenic polypeptide and the second immunogenic polypeptide are separately fused to a first and a second autotransporter, and optionally the first autotransporter is different from the second autotransporter.

15. The preparation of claim 12, wherein the first immunogenic polypeptide is from a pathogen and the second immunogenic polypeptide is an adjuvant polypeptide.

16. The preparation of claim 15, wherein the adjuvant polypeptide is fused to a second autotransporter or wherein the adjuvant polypeptide is the extracellular portion of the autotransporter or a part thereof.

17. The preparation of claim 1, comprising a mixture of non-viable bacterium cells comprising a first immunogenic polypeptide and non-viable bacterium cells comprising a second immunogenic polypeptide, wherein the first immunogenic polypeptide is different from the second immunogenic polypeptide.

18. The preparation of claim 16, wherein the first immunogenic polypeptide and the second immunogenic polypeptide are from the same pathogen or are from different pathogens.

19. The preparation of claim 16, wherein the first immunogenic polypeptide is from a pathogen and the second immunogenic polypeptide is an adjuvant polypeptide.

20. The preparation of claim 1, wherein the preparation is an effervescent powder.

21. A process for preparing the non-viable bacterium cells of the preparation of claim 1, comprising:
(a) cultivating viable bacterium cells in a culture medium wherein the bacterium cells are transformed or transfected with a recombinant nucleic acid molecule comprising:
(i) a portion encoding a signal peptide,
(ii) a portion encoding the immunogenic polypeptide to be dis-played,
(iii) a portion encoding a transmembrane linker, and
(iv) a portion encoding the transporter domain of an autotransporter protein; under conditions where the recombinant nucleic acid molecule is expressed;
(b) obtaining bacterium cells wherein an immunogenic polypeptide fused to an autotransporter protein comprising transmembrane linker and an autotransporter domain encoded by the recombinant nucleic acid molecule of (a) is displayed on the surface of the cells; and
(c) subjecting the bacterium cells of (b) to conditions where non-viable bacterium cells are obtained,
wherein in step (c) the bacterium cells are rendered non-viable by lyophilization.

22. The process of claim 21, wherein step (c) comprises lyophilizing the bacterium cells of (b) in the presence of a phosphate ion containing aqueous medium, e.g., a phosphate-buffered saline (PBS) containing aqueous medium.

23. The process of claim 21, wherein non-viable bacterium cells comprising a first immunogenic polypeptide and non-viable bacterium cells comprising a second immunogenic polypeptide are mixed, wherein the non-viable bacterium cells comprising a first immunogenic polypeptide and the non-viable bacterium cells comprising a second immunogenic polypeptide are separately obtained in step (b).

24. The process of claim 23, wherein the non-viable bacterium cells are mixed before lyophilization or are mixed after lyophilization in step (c).

25. The preparation of claim 1 for use in medicine.

26. An oral vaccine comprising the preparation of claim 1.


SEQUENCE LISTING (12 sequences)

SEQ ID NO: 1 - DNA, 2916 bp, synthetic construct
  source 1..2916: mol_type = other DNA
  misc_feature 1..81: note = CtxB signal peptide sequence
  misc_feature 82..99: note = 6xHis Tag
  misc_feature 100..111: note = Factor Xa cleavage site
  misc_feature 112..117: note = XhoI restriction site
  misc_feature 118..1371: note = Nucleocapsid protein
  misc_feature 1372..1377: note = KpnI restriction site
  misc_feature 1378..1389: note = OmpT cleavage site
  misc_feature 1390..1401: note = Factor Xa cleavage site
  misc_feature 1405..1419: note = PEYFK-Tag
  misc_feature 1420..1449: note = Myc-Tag
  misc_feature 1450..2061: note = EhaA Linker
  misc_feature 2062..2916: note = Beta-Barrel
  CDS 1..2916: protein_id = 2; translation = MIKLKFGVFFTVLLSSAYAHGTPQNITHHHHHHIEGRLESDNGP QNQRNAPRITFGGPSDSTGSNQNGERSGARSKQRRPQGLPNNTASWFTALTQHGKEDL KFPRGQGVPINTNSSPDDQIGYYRRATRRIRGGDGKMKDLSPRWYFYYLGTGPEAGLP YGANKDGIIWVATEGALNTPKDHIGTRNPANNAAIVLQLPQGTTLPKGFYAEGSRGGS QASSRSSSRSRNSSRNSTPGSSRGTSPARMAGNGGDAALALLLLDRLNQLESKMSGKG QQQQGQTVTKKSAAEASKKPRQKRTATKAYNVTQAFGRRGPEQTQGNFGDQELIRQGT DYKHWPQIAQFAPSASAFFGMSRIGMEVTPSGTWLTYTGAIKLDDKDPNFKDQVILLN KHIDAYKTFPPTEPKKDKKKKADETQALPQRQKKQQTVTLLPAADLDDFSKQLQQSMS SADSTQAGTARRAIEGRIPEYFKEQKLISEEDLLTNNGTLMTGMSGQQAGNVLVVKGN YHGNNGQLVMNTVLNGDDSVTDKLVVEGDTSGTTAVTVNNAGGTGAKTLNGIELIHVD GKSEGEFVQAGRIVAGAYDYTLARGQGANSGNWYLTSGSDSPELQPEPDPMPNPEPNP NPEPNPNPTPTPGPDLNVDNDLRPEAGSYIANLAAANTMFTTRLHERLGNTYYTDMVT GEQKQTTMWMRHEGGHNKWRDGSGQLKTQSNRYVLQLGGDVAQWSQNGSDRWHVGVMA GYGNSDSKTISSRTGYRAKASVNGYSTGLYATWYADDESRNGAYLDSWAQYSWFDNTV KGDDLQSESYKSKGFTASLEAGYKHKLAEFNGSQGTRNEWYVQPQAQVTWMGVKADKH RESNGTLVHSNGDGNVQTRLGVKTWLKSHHKMDDGKSREFQPFVEVNWLHNSKDFSTS MDGVSVTQDGARNIAEIKTGVEGQLNANLNVWGNVGVQVADRGYNDTSAMVGIKWQF
atgatcaaactgaaattcggcgtcttcttcaccgtactgctgtcctctgcttacgctcacggtactccgcagaacatcacccaccaccatcaccatcatatcgaaggtcgtctcgagtcggacaatggtccccaaaatcaacgtaatgctccccgtatcactttcggtgggccatcagactctacagggagtaatcaaaatggagagcgttctggtgcccgttccaagcaacgtcgtcctcaaggtcttcctaataatacggcatcctggttcacggccttgacacaacacggtaaggaagacttgaagttcccccgtggtcaaggagtaccaatcaatacgaattcatctccagacgaccaaatcggttactaccgtcgtgccacgcgtcgtatccgtggtggggacggtaagatgaaggacttgtcgcctcgttggtacttctactacctcggaactggtcctgaggccggtttgccttacggagcaaataaggacgggatcatctgggtcgcaacagagggagcattaaatacaccaaaggaccacatcggaacgcgtaatccagcaaataatgccgccatcgtactccaattacctcaaggaacaacgttgccaaagggattctacgccgagggatcccgtggtgggtctcaagcaagtagtcgttcatcaagtcgttcgcgtaattcttcccgtaatagtacacctggatccagtcgtggtacatcgcctgcccgtatggccggtaatggtggtgacgccgcattagccttgcttttgttagaccgtctcaatcaattagagtcaaagatgtcgggtaagggtcaacaacaacaaggacaaacagtaactaagaagtccgccgccgaggcctctaagaagccccgtcaaaagcgtacggctactaaggcatacaatgtcacacaagccttcggacgtcgtgggcccgagcaaactcaaggtaatttcggggaccaagagttgatccgtcaaggaactgactacaagcactggccacaaatcgcacaattcgccccatccgcctctgccttcttcggtatgtcacgtatcggtatggaagttactccctcgggaacgtggttaacgtacacgggtgccatcaagctcgacgacaaggacccaaatttcaaggaccaagtcatccttctcaataagcacatcgacgcctacaagactttcccacccactgagcctaagaaggacaagaagaagaaggcagacgagacacaagctttaccccaacgtcaaaagaagcaacaaactgttactctcttgcccgccgccgacttggacgacttctcgaagcaattacaacaaagtatgtcgtcagctgactccacgcaagctggtaccgctcgtcgtgctattgagggccgcatcccggaatactttaaagaacaaaaactcatctcagaagaggatctgctgaccaacaatggcacgctgatgacgggtatgagcggtcaacaagcgggtaacgttctggttgttaagggcaattaccatggcaataacggccagctggtcatgaacacggttctgaacggcgatgatagcgtgaccgacaagctggtggtcgagggcgacacctctggtacgaccgcagtgacggtgaataatgcaggcggtacgggtgccaaaaccctgaacggtattgagttgatccacgttgacggtaagagcgagggcgagtttgtgcaggcaggccgcattgttgctggcgcttatgactatacgctggcccgtggtcagggcgcgaatagcggtaactggtatctgaccagcggctccgactccccggaactgcaaccggagcctgatccgatgccgaatccggagccaaacccgaacccggaaccgaacccaaatccgaccccgactccgggtccggacttgaacgttgataacgacctgcgtccggaggccggttcgtacatcgcgaacctggcagcggccaatacgatgtttacgacccgtctgcacgaacgcctgggtaatacctactataccgatatggtcactggtgaacagaaacaaaccaccatgtggatgcgccacgagggtggtcacaataagtggcgcgacggtagcggccagttgaaaacccagagcaatcgctacgttctgcaattgggcggtgatgtggcgcaatggagccaaaacggcagcgaccgttggcatgtcggtgtgatggcaggttacggcaacagcgacagcaagaccatctccagccgtaccggttaccgtgcgaaggcaagcgtcaacggttacagcaccggcctgtatgccacctggtatgctgatgatgagagccgcaacggtgcttacttggacagctgggcacagtattcttggttcgataatacggtgaaaggcgacgacctgcagagcgaaagctacaaatcgaaaggtttcaccgcgagcctggaagccggctataagcacaaactggcggaattcaatggcagccagggtactcgtaacgaatggtacgttcaaccgcaggcgcaagtcacttggatgggcgttaaggcggataaacaccgtgagagcaacggtacgttggtgcatagcaacggtgatggtaatgtccaaacccgtctgggtgtgaaaacgtggctgaagtcccatcacaaaatggacgacggtaaatctcgtgaatttcagccgttcgtggaagttaactggctgcataatagcaaggatttcagcacgagcatggatggtgtctccgttacccaggacggcgcacgtaacattgcggagatcaagaccggcgtcgagggtcagctgaatgcgaatctgaatgtttggggtaacgtgggtgttcaagtagcggaccgtggttacaatgataccagcgcgatggtgggtattaagtggcagttttaa

SEQ ID NO: 2 - protein, 971 aa, synthetic construct
  REGION 1..27: note = CtxB signal peptide sequence
  REGION 28..33: note = 6xHis Tag
  REGION 34..37: note = Factor Xa cleavage site
  REGION 38..39: note = XhoI restriction site
  REGION 40..457: note = Nucleocapsid protein
  REGION 458..459: note = KpnI restriction site
  REGION 460..463: note = OmpT cleavage site
  REGION 464..467: note = Factor Xa cleavage site
  REGION 469..473: note = PEYFK-Tag
  REGION 474..483: note = Myc-Tag
  REGION 484..687: note = EhaA Linker
  REGION 688..971: note = Beta-Barrel
  REGION 1..971: note = nucleocapsid autotransporter fusion protein
MIKLKFGVFFTVLLSSAYAHGTPQNITHHHHHHIEGRLESDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGTARRAIEGRIPEYFKEQKLISEEDLLTNNGTLMTGMSGQQAGNVLVVKGNYHGNNGQLVMNTVLNGDDSVTDKLVVEGDTSGTTAVTVNNAGGTGAKTLNGIELIHVDGKSEGEFVQAGRIVAGAYDYTLARGQGANSGNWYLTSGSDSPELQPEPDPMPNPEPNPNPEPNPNPTPTPGPDLNVDNDLRPEAGSYIANLAAANTMFTTRLHERLGNTYYTDMVTGEQKQTTMWMRHEGGHNKWRDGSGQLKTQSNRYVLQLGGDVAQWSQNGSDRWHVGVMAGYGNSDSKTISSRTGYRAKASVNGYSTGLYATWYADDESRNGAYLDSWAQYSWFDNTVKGDDLQSESYKSKGFTASLEAGYKHKLAEFNGSQGTRNEWYVQPQAQVTWMGVKADKHRESNGTLVHSNGDGNVQTRLGVKTWLKSHHKMDDGKSREFQPFVEVNWLHNSKDFSTSMDGVSVTQDGARNIAEIKTGVEGQLNANLNVWGNVGVQVADRGYNDTSAMVGIKWQF

SEQ ID NO: 3 - DNA, 5256 bp, synthetic construct
  source 1..5256: mol_type = other DNA
  misc_feature 1..81: note = CtxB signal peptide sequence
  misc_feature 82..99: note = 6xHis Tag
  misc_feature 100..111: note = Factor Xa cleavage site
  misc_difference 112..117: note = XhoI restriction site
  misc_feature 118..3711: note = Spike protein
  misc_feature 3712..3717: note = KpnI restriction site
  misc_feature 3718..3729: note = OmpT cleavage site
  misc_feature 3730..3741: note = Factor Xa cleavage site
  misc_feature 3745..3759: note = PEYFK-Tag
  misc_feature 3760..3789: note = Myc-Tag
  misc_feature 3790..4401: note = EhaA Linker
  misc_feature 4402..5256: note = Beta-Barrel
  CDS 1..5256: protein_id = 4; translation = MIKLKFGVFFTVLLSSAYAHGTPQNITHHHHHHIEGRLEVNLTT RTQLPPAYTNSFTRGVYYPDKVFRSSVLHSTQDLFLPFFSNVTWFHAIHVSGTNGTKR FDNPVLPFNDGVYFASTEKSNIIRGWIFGTTLDSKTQSLLIVNNATNVVIKVCEFQFC NDPFLGVYYHKNNKSWMESEFRVYSSANNCTFEYVSQPFLMDLEGKQGNFKNLREFVF KNIDGYFKIYSKHTPINLVRDLPQGFSALEPLVDLPIGINITRFQTLLALHRSYLTPG DSSSGWTAGAAAYYVGYLQPRTFLLKYNENGTITDAVDCALDPLSETKCTLKSFTVEK GIYQTSNFRVQPTESIVRFPNITNLCPFGEVFNATRFASVYAWNRKRISNCVADYSVL YNSASFSTFKCYGVSPTKLNDLCFTNVYADSFVIRGDEVRQIAPGQTGKIADYNYKLP DDFTGCVIAWNSNNLDSKVGGNYNYLYRLFRKSNLKPFERDISTEIYQAGSTPCNGVE GFNCYFPLQSYGFQPTNGVGYQPYRVVVLSFELLHAPATVCGPKKSTNLVKNKCVNFN FNGLTGTGVLTESNKKFLPFQQFGRDIADTTDAVRDPQTLEILDITPCSFGGVSVITP GTNTSNQVAVLYQDVNCTEVPVAIHADQLTPTWRVYSTGSNVFQTRAGCLIGAEHVNN SYECDIPIGAGICASYQTQTNSPRRARSVASQSIIAYTMSLGAENSVAYSNNSIAIPT NFTISVTTEILPVSMTKTSVDCTMYICGDSTECSNLLLQYGSFCTQLNRALTGIAVEQ DKNTQEVFAQVKQIYKTPPIKDFGGFNFSQILPDPSKPSKRSFIEDLLFNKVTLADAG FIKQYGDCLGDIAARDLICAQKFNGLTVLPPLLTDEMIAQYTSALLAGTITSGWTFGA GAALQIPFAMQMAYRFNGIGVTQNVLYENQKLIANQFNSAIGKIQDSLSSTASALGKL QDVVNQNAQALNTLVKQLSSNFGAISSVLNDILSRLDPPEAEVQIDRLITGRLQSLQT YVTQQLIRAAEIRASANLAATKMSECVLGQSKRVDFCGKGYHLMSFPQSAPHGVVFLH VTYVPAQEKNFTTAPAICHDGKAHFPREGVFVSNGTHWFVTQRNFYEPQIITTDNTFV SGNCDVVIGIVNNTVYDPLQPELDSFKEELDKYFKNHTSPDVDLGDISGINASVVNIQ KEIDRLNEVAKNLNESLIDLQELGKYEQYIKWPGTARRAIEGRIPEYFKEQKLISEED LLTNNGTLMTGMSGQQAGNVLVVKGNYHGNNGQLVMNTVLNGDDSVTDKLVVEGDTSG TTAVTVNNAGGTGAKTLNGIELIHVDGKSEGEFVQAGRIVAGAYDYTLARGQGANSGN WYLTSGSDSPELQPEPDPMPNPEPNPNPEPNPNPTPTPGPDLNVDNDLRPEAGSYIAN LAAANTMFTTRLHERLGNTYYTDMVTGEQKQTTMWMRHEGGHNKWRDGSGQLKTQSNR YVLQLGGDVAQWSQNGSDRWHVGVMAGYGNSDSKTISSRTGYRAKASVNGYSTGLYAT WYADDESRNGAYLDSWAQYSWFDNTVKGDDLQSESYKSKGFTASLEAGYKHKLAEFNG SQGTRNEWYVQPQAQVTWMGVKADKHRESNGTLVHSNGDGNVQTRLGVKTWLKSHHKM DDGKSREFQPFVEVNWLHNSKDFSTSMDGVSVTQDGARNIAEIKTGVEGQLNANLNVW GNVGVQVADRGYNDTSAMVGIKWQF
atgatcaaactgaaattcggcgtcttcttcaccgtactgctgtcctctgcttacgctcacggtactccgcagaacatcacccaccaccatcaccatcatatcgaaggtcgtctcgaggttaatttaactacgcgtacgcaactcccgccagcctacacaaattccttcacgcgtggggtctactaccccgacaaggtattccgttccagtgttttgcactcgacgcaagaccttttcttgcccttcttctcgaatgtcacatggttccacgccatccacgtatctggtacaaatggaactaagcgtttcgacaatcccgtcttgcctttcaatgacggagtctacttcgccagtacggagaagtccaatatcatccgtggttggatcttcggtacaacattagactcgaagactcaatcgttacttatcgtcaataatgctactaatgttgttatcaaggtatgtgagttccaattctgtaatgaccccttcttgggtgtctactaccacaagaataataagtcttggatggagtcagagttccgtgtttactcatcagccaataattgtacattcgagtacgtatcccaacctttcttaatggacttagagggaaagcaagggaatttcaagaatttacgtgagttcgttttcaagaatatcgacggatacttcaagatctactcgaagcacacacctatcaatttggtccgtgaccttccccaaggtttctcagctttggagcccttagttgacctgcccatcggaatcaatatcacgcgtttccaaacgcttctcgcacttcaccgttcgtacttgacgcccggtgactcctcatcgggttggacggcaggagcagcagcctactacgttggataccttcaacctcgtacgttcttgttaaagtacaatgagaatggtactatcacagacgctgtagactgtgctttagacccattatcggagacaaagtgtacactcaagtctttcacggtagagaaggggatctaccaaacgagtaatttccgtgttcaaccaactgagtctatcgttcgtttcccaaatatcactaatttgtgtccattcggtgaggtcttcaatgccacacgtttcgcaagtgtttacgcctggaatcgtaagcgtatctcaaattgtgtcgccgactactcagttctctacaattcagcatcattctcaacgttcaagtgttacggagtttcacctacgaagttaaatgacttgtgtttcactaatgtatacgcagacagtttcgtcatccgtggagacgaggttcgtcaaatcgctccaggacaaacaggaaagatcgccgactacaattacaagttgccagacgacttcactgggtgtgttatcgcatggaattctaataatcttgactcgaaggtcggtggtaattacaattacctttaccgtctcttccgtaagtcgaatttgaagcctttcgagcgtgacatctcaactgagatctaccaagctgggtcgacgccatgtaatggtgtagaggggttcaattgttacttccccttacaatcatacgggttccaacccactaatggagtaggttaccaaccataccgtgtagtagttttgtcgttcgagttacttcacgcccccgctacggtttgtggtcctaagaagtcgactaatctcgtcaagaataagtgtgtcaatttcaatttcaatggactcacgggtacaggtgtccttacggagagtaataagaagttcttgccattccaacaattcggtcgtgacatcgccgacactactgacgcagttcgtgacccacaaactttggagatcctcgacatcacaccatgttctttcggcggtgtatccgtcatcactcccggaacaaatacgtcgaatcaagtagcagtactctaccaagacgtaaattgtacggaagttcccgttgccatccacgctgaccaattaactccaacgtggcgtgtttactcaacagggagtaatgtattccaaacgcgtgccggttgtttgatcggagccgagcacgtcaataattcatacgagtgtgacatccctatcggggccggaatctgtgccagttaccaaacgcaaacgaatagtccacgtcgtgcacgttcggtcgcctctcaatcaatcatcgcctacacgatgtcgctcggggccgagaattcggtagcctactcgaataattcgatcgcaatccctacgaatttcacgatctcagttacaactgagatccttccagtttcaatgacgaagacatcggtcgactgtacgatgtacatctgtggggactcgactgagtgttcgaatcttttattgcaatacggttcattctgtactcaattgaatcgtgccttaactggaatcgcagtagagcaagacaagaatacgcaagaggttttcgctcaagtaaagcaaatctacaagacaccacctatcaaggacttcggtggtttcaatttctcccaaatcttacctgacccttcgaagccatcaaagcgttcattcatcgaggaccttttgttcaataaggtcactttggctgacgccggtttcatcaagcaatacggagactgtcttggagacatcgcagcccgtgaccttatctgtgcacaaaagttcaatggtttaacagtactcccgcctttattgacggacgagatgatcgcacaatacacttctgctcttcttgcagggacgatcacgtcagggtggactttcggtgccggtgcagccttacaaatccctttcgccatgcaaatggcctaccgtttcaatggtatcggtgttacgcaaaatgtattatacgagaatcaaaagttgatcgctaatcaattcaattctgctatcggcaagatccaagactcattatccagcactgcttccgccttgggaaagctccaagacgtcgtcaatcaaaatgcccaagcattgaatacattggttaagcaactcagttcgaatttcggagcaatctcgtcagttttaaatgacatcttgtcgcgtctcgacccgcctgaggcagaggtccaaatcgaccgtttaatcacaggtcgtcttcaatcgttgcaaacttacgtcacacaacaactcatccgtgccgcagagatccgtgcatccgctaatcttgccgccacgaagatgtcggagtgtgtcctcggtcaatcgaagcgtgtagacttctgtggtaagggttaccaccttatgtccttcccccaatccgcacctcacggtgttgttttcttacacgtaacgtacgtacctgcccaagagaagaatttcactactgcacctgccatctgtcacgacggaaaggcccacttccctcgtgagggtgttttcgtctccaatgggacacactggttcgttacacaacgtaatttctacgagcctcaaatcatcactactgacaatacattcgtttctggtaattgtgacgtagttatcgggatcgtaaataatactgtttacgaccctttacaaccagagttggacagtttcaaagaggagttagacaagtacttcaagaatcacacgtcacctgacgtcgaccttggtgacatctcgggtatcaatgcctcagtagttaatatccaaaaggagatcgaccgtttgaatgaggtagccaagaatttgaatgagtcccttatcgaccttcaagagctcggtaagtacgagcaatacatcaagtggcctggtaccgctcgtcgtgctattgagggccgcatcccggaatactttaaagaacaaaaactcatctcagaagaggatctgctgaccaacaatggcacgctgatgacgggtatgagcggtcaacaagcgggtaacgttctggttgttaagggcaattaccatggcaataacggccagctggtcatgaacacggttctgaacggcgatgatagcgtgaccgacaagctggtggtcgagggcgacacctctggtacgaccgcagtgacggtgaataatgcaggcggtacgggtgccaaaaccctgaacggtattgagttgatccacgttgacggtaagagcgagggcgagtttgtgcaggcaggccgcattgttgctggcgcttatgactatacgctggcccgtggtcagggcgcgaatagcggtaactggtatctgaccagcggctccgactccccggaactgcaaccggagcctgatccgatgccgaatccggagccaaacccgaacccggaaccgaacccaaatccgaccccgactccgggtccggacttgaacgttgataacgacctgcgtccggaggccggttcgtacatcgcgaacctggcagcggccaatacgatgtttacgacccgtctgcacgaacgcctgggtaatacctactataccgatatggtcactggtgaacagaaacaaaccaccatgtggatgcgccacgagggtggtcacaataagtggcgcgacggtagcggccagttgaaaacccagagcaatcgctacgttctgcaattgggcggtgatgtggcgcaatggagccaaaacggcagcgaccgttggcatgtcggtgtgatggcaggttacggcaacagcgacagcaagaccatctccagccgtaccggttaccgtgcgaaggcaagcgtcaacggttacagcaccggcctgtatgccacctggtatgctgatgatgagagccgcaacggtgcttacttggacagctgggcacagtattcttggttcgataatacggtgaaaggcgacgacctgcagagcgaaagctacaaatcgaaaggtttcaccgcgagcctggaagccggctataagcacaaactggcggaattcaatggcagccagggtactcgtaacgaatggtacgttcaaccgcaggcgcaagtcacttggatgggcgttaaggcggataaacaccgtgagagcaacggtacgttggtgcatagcaacggtgatggtaatgtccaaacccgtctgggtgtgaaaacgtggctgaagtcccatcacaaaatggacgacggtaaatctcgtgaatttcagccgttcgtggaagttaactggctgcataatagcaaggatttcagcacgagcatggatggtgtctccgttacccaggacggcgcacgtaacattgcggagatcaagaccggcgtcgagggtcagctgaatgcgaatctgaatgtttggggtaacgtgggtgttcaagtagcggaccgtggttacaatgataccagcgcgatggtgggtattaagtggcagttttaa

SEQ ID NO: 4 - protein, 1751 aa, synthetic construct
  REGION 1..1751: note = spike autotransporter fusion protein
  REGION 1..27: note = CtxB signal peptide sequence
  REGION 28..33: note = 6xHis Tag
  REGION 34..37: note = Factor Xa cleavage site
  REGION 38..39: note = XhoI restriction site
  REGION 40..1237: note = Spike protein
  REGION 1238..1339: note = KpnI restriction site
  REGION 1240..1243: note = OmpT cleavage site
  REGION 1244..1247: note = Factor Xa cleavage site
  REGION 1249..1253: note = PEYFK-Tag
  REGION 1254..1263: note = Myc-Tag
  REGION 1264..1467: note = EhaA Linker
  REGION 1468..1751: note = Beta-Barrel
MIKLKFGVFFTVLLSSAYAHGTPQNITHHHHHHIEGRLEVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGTARRAIEGRIPEYFKEQKLISEEDLLTNNGTLMTGMSGQQAGNVLVVKGNYHGNNGQLVMNTVLNGDDSVTDKLVVEGDTSGTTAVTVNNAGGTGAKTLNGIELIHVDGKSEGEFVQAGRIVAGAYDYTLARGQGANSGNWYLTSGSDSPELQPEPDPMPNPEPNPNPEPNPNPTPTPGPDLNVDNDLRPEAGSYIANLAAANTMFTTRLHERLGNTYYTDMVTGEQKQTTMWMRHEGGHNKWRDGSGQLKTQSNRYVLQLGGDVAQWSQNGSDRWHVGVMAGYGNSDSKTISSRTGYRAKASVNGYSTGLYATWYADDESRNGAYLDSWAQYSWFDNTVKGDDLQSESYKSKGFTASLEAGYKHKLAEFNGSQGTRNEWYVQPQAQVTWMGVKADKHRESNGTLVHSNGDGNVQTRLGVKTWLKSHHKMDDGKSREFQPFVEVNWLHNSKDFSTSMDGVSVTQDGARNIAEIKTGVEGQLNANLNVWGNVGVQVADRGYNDTSAMVGIKWQF

SEQ ID NO: 5 - DNA, 2412 bp, synthetic construct
  source 1..2412: mol_type = other DNA
  CDS 1..2412: protein_id = 6; translation = MIKLKFGVFFTVLLSSAYAHGTPQNITHHHHHHIEGRLERVQPT ESIVRFPNITNLCPFGEVFNATRFASVYAWNRKRISNCVADYSVLYNSASFSTFKCYG VSPTKLNDLCFTNVYADSFVIRGDEVRQIAPGQTGKIADYNYKLPDDFTGCVIAWNSN NLDSKVGGNYNYLYRLFRKSNLKPFERDISTEIYQAGSTPCNGVEGFNCYFPLQSYGF QPTNGVGYQPYRVVVLSFELLHAPATVCGPKKSTNLVKNKCVNFGYIPEAPRDGQAYV RKDGEWVLLSTFLGTARRAIEGRIPEYFKEQKLISEEDLLTNNGTLMTGMSGQQAGNV LVVKGNYHGNNGQLVMNTVLNGDDSVTDKLVVEGDTSGTTAVTVNNAGGTGAKTLNGI ELIHVDGKSEGEFVQAGRIVAGAYDYTLARGQGANSGNWYLTSGSDSPELQPEPDPMP NPEPNPNPEPNPNPTPTPGPDLNVDNDLRPEAGSYIANLAAANTMFTTRLHERLGNTY YTDMVTGEQKQTTMWMRHEGGHNKWRDGSGQLKTQSNRYVLQLGGDVAQWSQNGSDRW HVGVMAGYGNSDSKTISSRTGYRAKASVNGYSTGLYATWYADDESRNGAYLDSWAQYS WFDNTVKGDDLQSESYKSKGFTASLEAGYKHKLAEFNGSQGTRNEWYVQPQAQVTWMG VKADKHRESNGTLVHSNGDGNVQTRLGVKTWLKSHHKMDDGKSREFQPFVEVNWLHNS KDFSTSMDGVSVTQDGARNIAEIKTGVEGQLNANLNVWGNVGVQVADRGYNDTSAMVG IKWQF
  misc_feature 1..81: note = CtxB signal peptide sequence
  misc_feature 92..99: note = 6xHis Tag
  misc_feature 100..111: note = Factor Xa cleavage site
  misc_feature 112..117: note = XhoI restriction site
  misc_feature 118..786: note = RBD protein
  misc_feature 787..867: note = Trimerization domain
  misc_feature 868..873: note = KpnI restriction site
  misc_feature 874..885: note = OmpT cleavage site
  misc_feature 886..897: note = Factor Xa cleavage site
  misc_feature 901..915: note = PEYFK-Tag
  misc_feature 916..945: note = Myc-Tag
  misc_feature 946..1557: note = EhaA Linker
  misc_feature 1558..2412: note = Beta-Barrel
atgatcaaactgaaattcggcgtcttcttcaccgtactgctgtcctctgcttacgctcacggtactccgcagaacatcacccaccaccatcaccatcatatcgaaggtcgtctcgagcgcgtgcaacctacagaatcgattgtgcgtttcccaaatattaccaatttgtgtccttttggagaggtgtttaatgcgactcgctttgcgagtgtgtatgcgtggaatcgcaagcgcattagcaattgtgtggcagattatagcgtcttatataattcagccagttttagcacgtttaagtgttatggagtgtcacctacgaagcttaatgatctctgttttacgaatgtgtatgccgatagttttgtaattcgcggtgatgaggtacgccaaattgcgcctggccaaacgggcaagattgcggattataattataagttaccagatgattttactggctgtgtgattgcgtggaatagcaataatctcgattccaaggtcggcggcaattataattatctctatcgcctgtttcgcaagtcaaatctcaagccttttgagcgcgatattagtacggagatttatcaagcaggcagtacaccatgtaacggcgtagagggtttcaactgttattttcccttacaaagctatggttttcaacctactaacggcgtcggctatcaaccatatcgcgtggtagtgctttcgtttgagttacttcatgcgcccgcgacagtgtgtggacctaagaagtccacgaatttagtgaagaataagtgtgtgaattttggctatattccggaagcgccgcgcgatggccaggcgtatgtgcgcaaagatggcgaatgggtgctgctgagcacctttctgggtaccgctcgtcgtgctattgagggccgcatcccggaatactttaaagaacaaaaactcatctcagaagaggatctgctgaccaacaatggcacgctgatgacgggtatgagcggtcaacaagcgggtaacgttctggttgttaagggcaattaccatggcaataacggccagctggtcatgaacacggttctgaacggcgatgatagcgtgaccgacaagctggtggtcgagggcgacacctctggtacgaccgcagtgacggtgaataatgcaggcggtacgggtgccaaaaccctgaacggtattgagttgatccacgttgacggtaagagcgagggcgagtttgtgcaggcaggccgcattgttgctggcgcttatgactatacgctggcccgtggtcagggcgcgaatagcggtaactggtatctgaccagcggctccgactccccggaactgcaaccggagcctgatccgatgccgaatccggagccaaacccgaacccggaaccgaacccaaatccgaccccgactccgggtccggacttgaacgttgataacgacctgcgtccggaggccggttcgtacatcgcgaacctggcagcggccaatacgatgtttacgacccgtctgcacgaacgcctgggtaatacctactataccgatatggtcactggtgaacagaaacaaaccaccatgtggatgcgccacgagggtggtcacaataagtggcgcgacggtagcggccagttgaaaacccagagcaatcgctacgttctgcaattgggcggtgatgtggcgcaatggagccaaaacggcagcgaccgttggcatgtcggtgtgatggcaggttacggcaacagcgacagcaagaccatctccagccgtaccggttaccgtgcgaaggcaagcgtcaacggttacagcaccggcctgtatgccacctggtatgctgatgatgagagccgcaacggtgcttacttggacagctgggcacagtattcttggttcgataatacggtgaaaggcgacgacctgcagagcgaaagctacaaatcgaaaggtttcaccgcgagcctggaagccggctataagcacaaactggcggaattcaatggcagccagggtactcgtaacgaatggtacgttcaaccgcaggcgcaagtcacttggatgggcgttaaggcggataaacaccgtgagagcaacggtacgttggtgcatagcaacggtgatggtaatgtccaaacccgtctgggtgtgaaaacgtggctgaagtcccatcacaaaatggacgacggtaaatctcgtgaatttcagccgttcgtggaagttaactggctgcataatagcaaggatttcagcacgagcatggatggtgtctccgttacccaggacggcgcacgtaacattgcggagatcaagaccggcgtcgagggtcagctgaatgcgaatctgaatgtttggggtaacgtgggtgttcaagtagcggaccgtggttacaatgataccagcgcgatggtgggtattaagtggcagttttaa

SEQ ID NO: 6 - protein, 803 aa, synthetic construct
  REGION 1..27: note = CtxB signal peptide sequence
  REGION 28..33: note = 6xHis Tag
  REGION 34..37: note = Factor Xa cleavage site
  REGION 38..39: note = XhoI restriction site
  REGION 40..262: note = RBD protein
  REGION 263..289: note = Trimerization domain
  REGION 290..291: note = KpnI restriction site
  REGION 292..295: note = OmpT cleavage site
  REGION 296..299: note = Factor Xa cleavage site
  REGION 301..305: note = PEYFK-Tag
  REGION 306..315: note = Myc-Tag
  REGION 316..519: note = EhaA Linker
  REGION 520..803: note = Beta-Barrel
  REGION 1..803: note = receptor binding domain autotransporter fusion protein including the trimerization domain
MIKLKFGVFFTVLLSSAYAHGTPQNITHHHHHHIEGRLERVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGYIPEAPRDGQAYVRKDGEWVLLSTFLGTARRAIEGRIPEYFKEQKLISEEDLLTNNGTLMTGMSGQQAGNVLVVKGNYHGNNGQLVMNTVLNGDDSVTDKLVVEGDTSGTTAVTVNNAGGTGAKTLNGIELIHVDGKSEGEFVQAGRIVAGAYDYTLARGQGANSGNWYLTSGSDSPELQPEPDPMPNPEPNPNPEPNPNPTPTPGPDLNVDNDLRPEAGSYIANLAAANTMFTTRLHERLGNTYYTDMVTGEQKQTTMWMRHEGGHNKWRDGSGQLKTQSNRYVLQLGGDVAQWSQNGSDRWHVGVMAGYGNSDSKTISSRTGYRAKASVNGYSTGLYATWYADDESRNGAYLDSWAQYSWFDNTVKGDDLQSESYKSKGFTASLEAGYKHKLAEFNGSQGTRNEWYVQPQAQVTWMGVKADKHRESNGTLVHSNGDGNVQTRLGVKTWLKSHHKMDDGKSREFQPFVEVNWLHNSKDFSTSMDGVSVTQDGARNIAEIKTGVEGQLNANLNVWGNVGVQVADRGYNDTSAMVGIKWQF

SEQ ID NO: 7 - DNA, 2331 bp, synthetic construct
  source 1..2331: mol_type = other DNA
  misc_feature 1..81: note = CtxB signal peptide sequence
  misc_feature 82..99: note = 6xHis Tag
  misc_feature 100..111: note = Factor Xa cleavage site
  misc_feature 112..117: note = XhoI restriction site
  misc_feature 118..786: note = RBD protein
  misc_feature 787..792: note = KpnI restriction site
  misc_feature 793..804: note = OmpT cleavage site
  misc_feature 805..816: note = Factor Xa cleavage site
  misc_feature 820..834: note = PEYFK-Tag
  misc_feature 835..864: note = Myc-Tag
  misc_feature 867..1476: note = EhaA Linker
  misc_feature 1477..2331: note = Beta-Barrel
  CDS 1..2331: protein_id = 8; translation = MIKLKFGVFFTVLLSSAYAHGTPQNITHHHHHHIEGRLERVQPT ESIVRFPNITNLCPFGEVFNATRFASVYAWNRKRISNCVADYSVLYNSASFSTFKCYG VSPTKLNDLCFTNVYADSFVIRGDEVRQIAPGQTGKIADYNYKLPDDFTGCVIAWNSN NLDSKVGGNYNYLYRLFRKSNLKPFERDISTEIYQAGSTPCNGVEGFNCYFPLQSYGF QPTNGVGYQPYRVVVLSFELLHAPATVCGPKKSTNLVKNKCVNFGTARRAIEGRIPEY FKEQKLISEEDLLTNNGTLMTGMSGQQAGNVLVVKGNYHGNNGQLVMNTVLNGDDSVT DKLVVEGDTSGTTAVTVNNAGGTGAKTLNGIELIHVDGKSEGEFVQAGRIVAGAYDYT LARGQGANSGNWYLTSGSDSPELQPEPDPMPNPEPNPNPEPNPNPTPTPGPDLNVDND LRPEAGSYIANLAAANTMFTTRLHERLGNTYYTDMVTGEQKQTTMWMRHEGGHNKWRD GSGQLKTQSNRYVLQLGGDVAQWSQNGSDRWHVGVMAGYGNSDSKTISSRTGYRAKAS VNGYSTGLYATWYADDESRNGAYLDSWAQYSWFDNTVKGDDLQSESYKSKGFTASLEA GYKHKLAEFNGSQGTRNEWYVQPQAQVTWMGVKADKHRESNGTLVHSNGDGNVQTRLG VKTWLKSHHKMDDGKSREFQPFVEVNWLHNSKDFSTSMDGVSVTQDGARNIAEIKTGV EGQLNANLNVWGNVGVQVADRGYNDTSAMVGIKWQF
atgatcaaactgaaattcggcgtcttcttcaccgtactgctgtcctctgcttacgctcacggtactccgcagaacatcacccaccaccatcaccatcatatcgaaggtcgtctcgagcgcgtgcaacctacagaatcgattgtgcgtttcccaaatattaccaatttgtgtccttttggagaggtgtttaatgcgactcgctttgcgagtgtgtatgcgtggaatcgcaagcgcattagcaattgtgtggcagattatagcgtcttatataattcagccagttttagcacgtttaagtgttatggagtgtcacctacgaagcttaatgatctctgttttacgaatgtgtatgccgatagttttgtaattcgcggtgatgaggtacgccaaattgcgcctggccaaacgggcaagattgcggattataattataagttaccagatgattttactggctgtgtgattgcgtggaatagcaataatctcgattccaaggtcggcggcaattataattatctctatcgcctgtttcgcaagtcaaatctcaagccttttgagcgcgatattagtacggagatttatcaagcaggcagtacaccatgtaacggcgtagagggtttcaactgttattttcccttacaaagctatggttttcaacctactaacggcgtcggctatcaaccatatcgcgtggtagtgctttcgtttgagttacttcatgcgcccgcgacagtgtgtggacctaagaagtccacgaatttagtgaagaataagtgtgtgaattttggtaccgctcgtcgtgctattgagggccgcatcccggaatactttaaagaacaaaaactcatctcagaagaggatctgctgaccaacaatggcacgctgatgacgggtatgagcggtcaacaagcgggtaacgttctggttgttaagggcaattaccatggcaataacggccagctggtcatgaacacggttctgaacggcgatgatagcgtgaccgacaagctggtggtcgagggcgacacctctggtacgaccgcagtgacggtgaataatgcaggcggtacgggtgccaaaaccctgaacggtattgagttgatccacgttgacggtaagagcgagggcgagtttgtgcaggcaggccgcattgttgctggcgcttatgactatacgctggcccgtggtcagggcgcgaatagcggtaactggtatctgaccagcggctccgactccccggaactgcaaccggagcctgatccgatgccgaatccggagccaaacccgaacccggaaccgaacccaaatccgaccccgactccgggtccggacttgaacgttgataacgacctgcgtccggaggccggttcgtacatcgcgaacctggcagcggccaatacgatgtttacgacccgtctgcacgaacgcctgggtaatacctactataccgatatggtcactggtgaacagaaacaaaccaccatgtggatgcgccacgagggtggtcacaataagtggcgcgacggtagcggccagttgaaaacccagagcaatcgctacgttctgcaattgggcggtgatgtggcgcaatggagccaaaacggcagcgaccgttggcatgtcggtgtgatggcaggttacggcaacagcgacagcaagaccatctccagccgtaccggttaccgtgcgaaggcaagcgtcaacggttacagcaccggcctgtatgccacctggtatgctgatgatgagagccgcaacggtgcttacttggacagctgggcacagtattcttggttcgataatacggtgaaaggcgacgacctgcagagcgaaagctacaaatcgaaaggtttcaccgcgagcctggaagccggctataagcacaaactggcggaattcaatggcagccagggtactcgtaacgaatggtacgttcaaccgcaggcgcaagtcacttggatgggcgttaaggcggataaacaccgtgagagcaacggtacgttggtgcatagcaacggtgatggtaatgtccaaacccgtctgggtgtgaaaacgtggctgaagtcccatcacaaaatggacgacggtaaatctcgtgaatttcagccgttcgtggaagttaactggctgcataatagcaaggatttcagcacgagcatggatggtgtctccgttacccaggacggcgcacgtaacattgcggagatcaagaccggcgtcgagggtcagctgaatgcgaatctgaatgtttggggtaacgtgggtgttcaagtagcggaccgtggttacaatgataccagcgcgatggtgggtattaagtggcagttttaa

SEQ ID NO: 8 - protein, 776 aa, synthetic construct
  REGION 1..776: note = receptor binding domain autotransporter fusion protein without trimerization domain
  REGION 1..27: note = CtxB signal peptide sequence
  REGION 28..33: note = 6xHis Tag
  REGION 34..37: note = Factor Xa cleavage site
  REGION 38..39: note = XhoI restriction site
  REGION 40..262: note = RBD protein
  REGION 263..264: note = KpnI restriction site
  REGION 265..268: note = OmpT cleavage site
  REGION 269..272: note = Factor Xa cleavage site
  REGION 274..278: note = PEYFK-Tag
  REGION 279..288: note = Myc-Tag
  REGION 289..492: note = EhaA Linker
  REGION 493..776: note = Beta-Barrel
MIKLKFGVFFTVLLSSAYAHGTPQNITHHHHHHIEGRLERVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGTARRAIEGRIPEYFKEQKLISEEDLLTNNGTLMTGMSGQQAGNVLVVKGNYHGNNGQLVMNTVLNGDDSVTDKLVVEGDTSGTTAVTVNNAGGTGAKTLNGIELIHVDGKSEGEFVQAGRIVAGAYDYTLARGQGANSGNWYLTSGSDSPELQPEPDPMPNPEPNPNPEPNPNPTPTPGPDLNVDNDLRPEAGSYIANLAAANTMFTTRLHERLGNTYYTDMVTGEQKQTTMWMRHEGGHNKWRDGSGQLKTQSNRYVLQLGGDVAQWSQNGSDRWHVGVMAGYGNSDSKTISSRTGYRAKASVNGYSTGLYATWYADDESRNGAYLDSWAQYSWFDNTVKGDDLQSESYKSKGFTASLEAGYKHKLAEFNGSQGTRNEWYVQPQAQVTWMGVKADKHRESNGTLVHSNGDGNVQTRLGVKTWLKSHHKMDDGKSREFQPFVEVNWLHNSKDFSTSMDGVSVTQDGARNIAEIKTGVEGQLNANLNVWGNVGVQVADRGYNDTSAMVGIKWQF

SEQ ID NO: 9 - protein, 195 aa, synthetic construct
  source 1..195: note = amino acid sequence of Pfs230D1M
SVLQSGALPSVGVDELDKIDLSYETTESGDTAVSEDSYDKYASQNTNKEYVCDFTDQLKPTESGPKVKKCEVKVNEPLIKVKIICPLKGSVEKLYDNIEYVPKKSPYVVLTKEETKLKEKLLSKLIYGLLISPTVNEKENNFKEGVIEFTLPPVVHKATVFYFICDNSKTEDDNKKGNRGIVEVYVEPYGNKING

SEQ ID NO: 10 - protein, 268 aa, synthetic construct
  source 1..268: note = amino acid sequence of RV21
GDRAAGQPAGDRAAGQPAGDRAAGQPAGDRAAGQPAGDRAAGQPAGDRADGQPAGDRADGQPAGDRADGQPAGDRADGQPAGNGAGGQAAANGAGNQPGANGAGGQAAANGAGNQPGANGAGGQAAANGAGDQPGANGAGDQPGANGADDQPGANGAGDQPGEDGAGNQPGANGAGDQPGGNGAGGQAAGGNAGGGQGQNNEGANAPNEKSVKEYLDKVRATVGTEWTPCSVTCGVGVRVRRRVNAANKKPEDLTLNDLETDVCTMDK

SEQ ID NO: 11 - protein, 507 aa, synthetic construct
  source 1..507: note = amino acid sequence of the L1 protein of human papilloma virus
MALWRPSDNTVYLPPPSVARVVNTDDYVTRTSIFYHAGSSRLLTVGNPYFRVPAGGGNKQDIPKVSAYQYRVFRVQLPDPNKFGLPDNSIYNPETQRLVWACAGVEIGRGQPLGVGLSGHPFYNKLDDTESSHAATSNVSEDVRDNVSVDYKQTQLCILGCAPAIGEHWAKGTACKSRPLSQGDCPPLELKNTVLEDGDMVDTGYGAMDFSTLQDTKCEVPLDICQSICKYPDYLQMSADPYGDSMFFCLRREQLFARHFWNRAGTMGDTVPQSLYIKGTGMRASPGSCVYSPSPSGSIVTSDSQLFNKPYWLHKAQGHNNGICWHNQLFVTVVDTTRSTNLTICASTQSPVPGQYDATKFKQYSRHVEEYDLQFIFQLCTITLTADVMSYIHSMNSSILEDWNFGVPPPPTTSLVDTYRFVQSVAITCQKDAAPAENKDPYDKLKFWNVDLKEKFSLDLDQYPLGRKFLVQAGLRRKPTIGPRKRSAPSATTSSKPAKRVRVRARK

SEQ ID NO: 12 - protein, 344 aa, synthetic construct
  source 1..344: note = amino acid sequence of N-terminal amino acids (22 kDa) of the BVDV E2 protein
YPHCKPGFLYAIAKNDEIGLLGAIGLTTQWYEYSDEMRLQDSTVEVWCKDGEVRYLTRCEREARYLAALHTRALPTSVVFEKIFDGKEQEDIVEMDDNFEFGLCPCDARPLIRGKFNTTLLNGPAFQMVCPIGWTGTVSCTLANKDTLATTVVRTYKRNSPFPHRQDCVTQKTIGEDLYNCALGGNWTCVPGDQLRYVAGPVESCKWCGHKFFKSEGLPHFPIGKCRLKNETGYRQVDETSCNRNGVAIVQSGLVKCRIGDTVVQVIAMDDKLGPMPCKPHEIIPSEGPVEKTACTFNYTRTLKNKYFEPRDNYFQQYMLKGEYQYWFDLEITDHHRDYFAEFL